(19) United States
(12) Patent Application Publication
(10) Pub. No.: US 2026/0193274 A1
(43) Pub. Date: Jul. 9, 2026

(54) AKT1 MODULATORS

(71) Applicant: Alterome Therapeutics, Inc., San Diego, CA (US)

(72) Inventors: Michael David BARTBERGER, Sherman Oaks, CA (US); Yi FAN, San Diego, CA (US); Eric Anthony MURPHY, San Marcos, CA (US); Xuefeng ZHU, San Diego, CA (US)

(21) Appl. No.: 19/128,308

(22) PCT Filed: Nov. 7, 2023
(86) PCT No.: PCT/US2023/078977

§ 371 Date: May 8, 2025

Related U.S. Application Data
(60) Provisional application No. 63/383,575, filed on Nov. 14, 2022.

(51) Int. Cl. C07D 519/00; A61K 31/444; A61K 31/4545; A61K 31/496; A61K 31/499; A61K 31/4995; A61K 31/551; C07B 59/00; C07D 471/04

(52) U.S. Cl. CPC C07D 519/00; A61K 31/444; A61K 31/4545; A61K 31/496; A61K 31/499; A61K 31/4995; A61K 31/551; C07B 59/002; C07D 471/04; C07B 2200/05

(57) ABSTRACT

Provided herein are inhibitors of AKT1, pharmaceutical compositions comprising the inhibitory compounds, and methods for using the AKT1 inhibitory compounds for the treatment of disease.

### CROSS REFERENCE

This application claims the benefit of U.S. Provisional Application No. 63/383,575 filed Nov. 14, 2022, which is incorporated herein by reference in its entirety.

### BACKGROUND

AKT is a protein kinase and mediates cell survival and proliferation by inhibiting pathways which promotes apoptosis. AKT signaling cascade dysfunction is observed in several cancer types and may be associated with tumor aggressiveness. Additionally, malfunction of AKT typically lead to enhanced proliferation, growth, survival, and resistance to apoptosis.

Pharmaceutical agents with the ability to modulate AKT1 activity would be useful in the treatment of disease, such as cancer.

### BRIEF SUMMARY OF THE INVENTION

Provided herein are inhibitors of AKT1, pharmaceutical compositions comprising said inhibitory compounds, and methods for using said inhibitory compounds for the treatment of disease.

One embodiment provides a compound having the structure of Formula (I), or a pharmaceutically acceptable salt or solvate thereof:

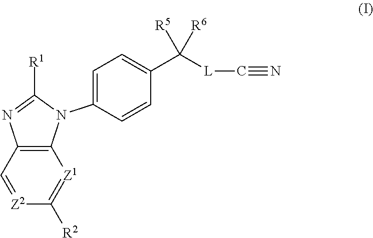

wherein:
Z¹ is N, C—H, or C—R³;
Z² is N, C—H, or C—R⁴;
R¹ is selected from hydrogen, halogen, —CN, optionally substituted C1-C6 alkyl, optionally substituted aryl, or optionally substituted heteroaryl;
R² is selected from hydrogen, halogen, —CN, optionally substituted C1-C6 alkyl, optionally substituted aryl, or optionally substituted heteroaryl;
R³ is selected from halogen, —CN, optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
L is selected from —N(R⁷)—, or a divalent radical selected from:

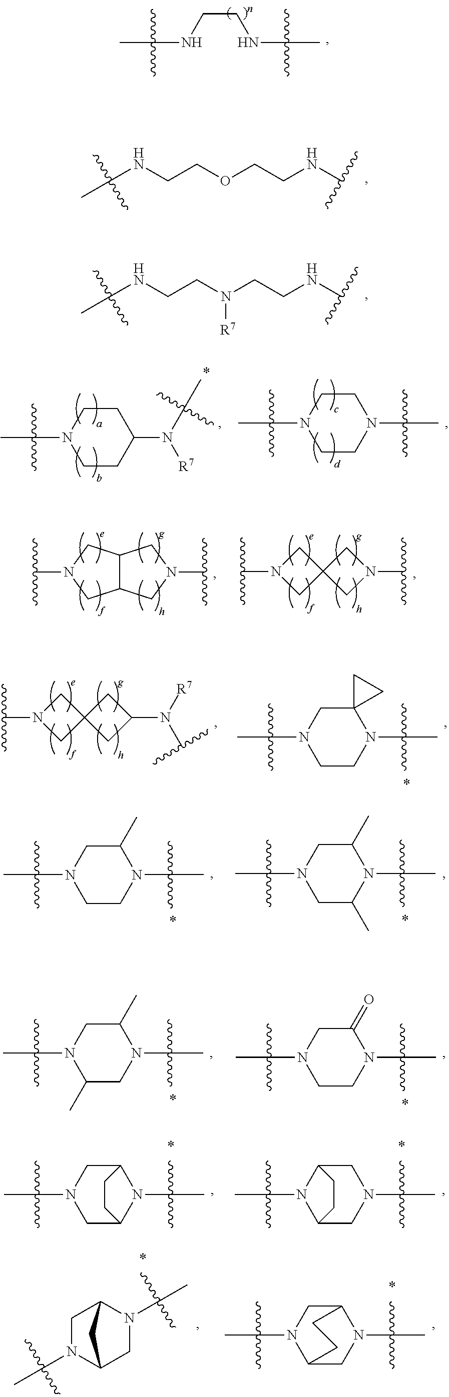

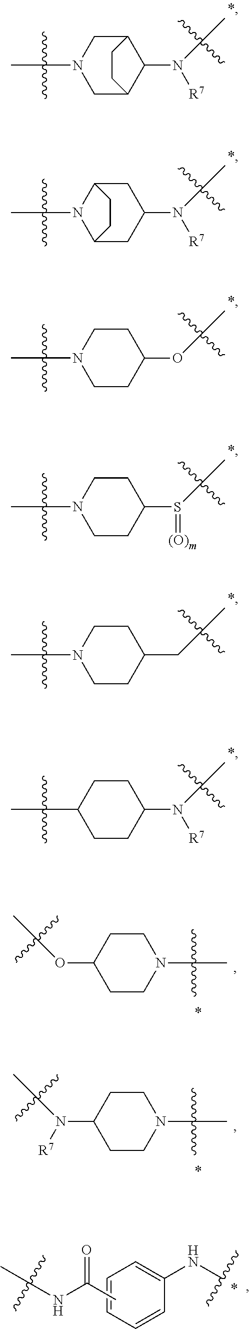

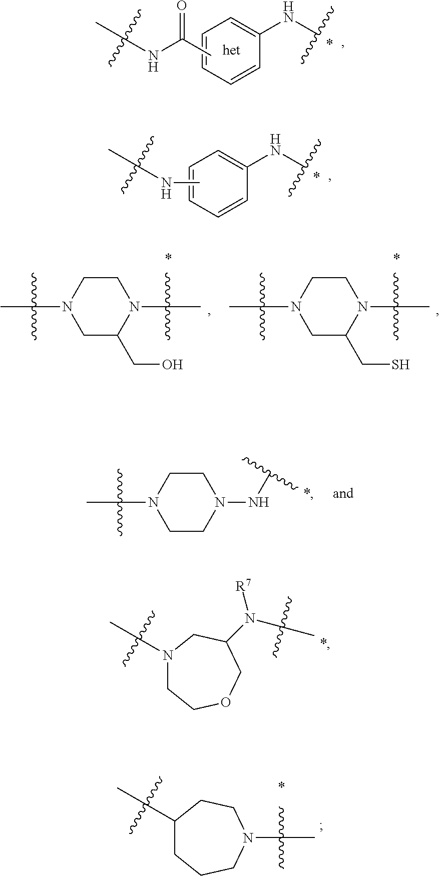

wherein the asterisk (*) indicates the bond to the cyanamide group;
a is 0, 1, 2, 3, or 4;
b is 0, 1, 2, 3, or 4;
c is 1, 2, 3, or 4;
d is 1, 2, 3, or 4;
e is 0, 1, 2, 3, or 4;
f is 0, 1, 2, 3, or 4; provided that e and f are not both 0;
g is 0, 1, 2, 3, or 4; provided that e and g are not both 0;
h is 0, 1, 2, 3, or 4; provided that g and h are not both 0; and provided that f and h are not both 0;
m is 0, 1, or 2;
n is 1, 2, or 3; and
R⁷ is hydrogen, optionally substituted C1-C6 alkyl, optionally substituted C3-C7 cycloalkyl, or optionally substituted heterocyclyl.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₈ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂(where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₈ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂(where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₈ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene).

Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₈ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂(where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) p-electron system in accordance with the Hickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}—R^{b}—OC(O)—N(R^{a})₂, —R^{b}—N(R^{a})₂, —R^{b}—C(O)R^{a}, —R—C(O)OR^{a}, —R^{b}—C(O)N(R^{a})₂, —R^{b}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) p-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

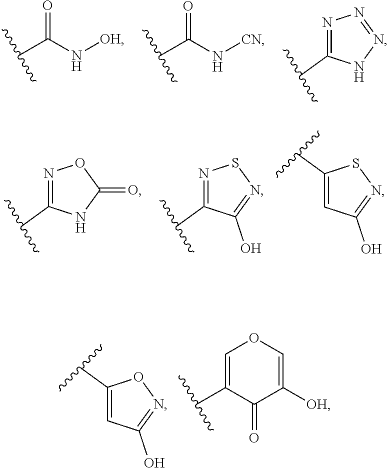

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

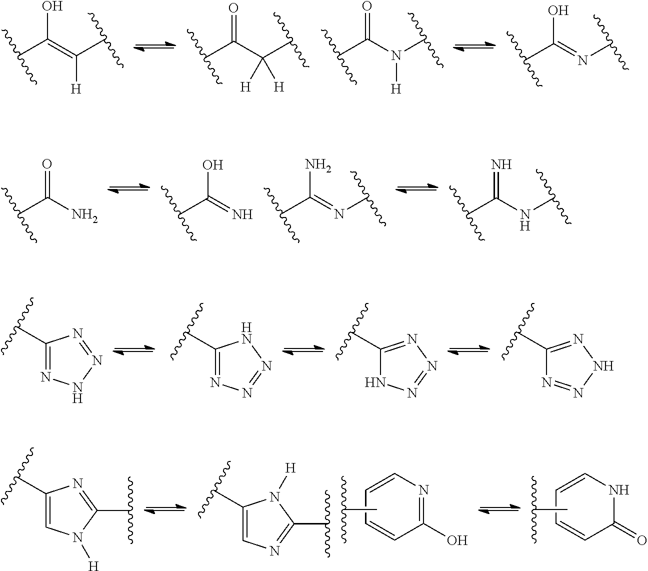

The compounds disclosed herein, in some embodiments, are used in different enriched isotopic forms, e.g., enriched in the content of ²H, ³H, ¹¹C, ³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with 18F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

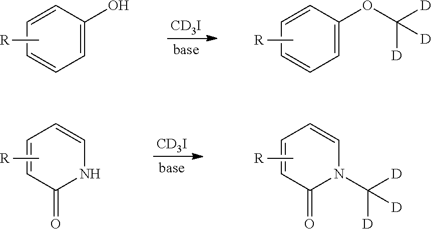

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

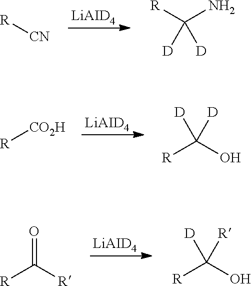

Deuterium gas and palladium catalyst are employed to reduce unsaturated carbon-carbon linkages and to perform a reductive substitution of aryl carbon-halogen bonds as illustrated, by way of example only, in the reaction schemes below.

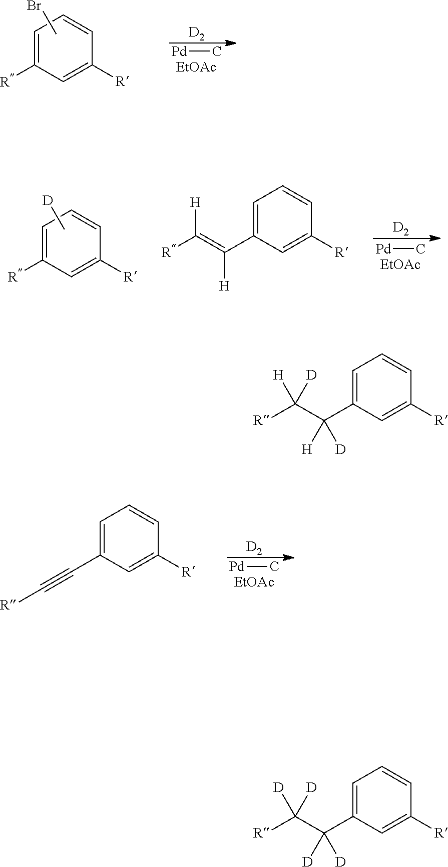

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the AKT1 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” means eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### AKT1 Protein and Function

AKT, also known as protein kinase B (PKB), is a serine/threonine protein kinase with three isoforms, AKT1, AKT2, and AKT3. While the isoforms are encoded by different genes, they are highly homologous at the protein level and share a conserved domain structure comprising an N-terminal pleckstrin homology (PH) domain, a kinase domain, and a C-terminal regulatory domain comprising a hydrophobic moiety, which includes the regulatory serine residue (Nitulescu, G. M. et al., Int J Oncol., 2018; 53(6): 2319-2331).

AKT proteins play a crucial role in major cellular functions including cell cycle progression, cell size, regulation of glucose metabolism, transcription, protein synthesis, genome stability, and neovascularization. AKT proteins can block apoptosis by inactivation of pro-apoptotic proteins, and mediate cellular growth factors, promoting cell survival. AKT is a major downstream effector of nuclear factor-kappaB (NfκB), which may link AKT signaling to the nucleus of a cell.

AKT1 is ubiquitously expressed, whereas AKT2 is primarily expressed in insulin-responsive tissues, and AKT3 is primarily expressed in brain and testes. A shared phosphorylation site of AKT in the catalytic domain corresponds to a threonine residue; specifically, Thr308 in AKT1, Thr309 in AKT2, and Thr305 in AKT3. A shared phosphorylation site in the C-terminus of the protein cis a serine residue; specifically, Ser473 in AKT1, Ser474 in AKT2, and Ser472 in AKT3.

AKT is a key downstream mediator of the phosphoinositide-3-kinase (PI3K) signaling pathway. PI3Ks are activated by different compounds. For example, PI3Kα, PI3Kβ, and PI3Kδ, are activated by extracellular ligands binding to a transmembrane glycoprotein with enzymatic activity, receptor tyrosine kinases (RTKs). In contrast, PI3Kγ is activated by G-protein-compound receptors (GPCRs) and by RAS family of GTPases.

The AKT cascade can be activated by RTKs and G-protein-compound receptors (GPCRs), along with other signals including integrins, B cell receptors, T cell receptors, and cytokine receptors.

### AKT1 Mechanism

AKT is activated by a second phosphorylation at the regulatory serine residue, Ser473. Known phosphorylating agents of AKT at Ser473 include, but are not limited to PDK-1, integrin-linked kinase (ILK), members of the PI3K-related kinase (PIKK) family, and mammalian target of rapamycin (mTOR) (Nitulescu, G. M. et al., Int J Oncol., 2018; 53(6): 2319-2331).

mTOR is a key component in the AKT signaling pathway, which is a downstream member of AKT and an important regulator for cell metabolism and growth. mTOR is also an activator which can directly phosphorylate AKT's regulatory serine residue, Ser473. mTOR forms a complex with rapamycin-insensitive companion of mTOR (RICTOR) (and other proteins) to form mTOR complex 2 (mTORC2), which can directly phosphorylate AKT Ser473. AKT can affect cell survival and growth because it can influence the tuberous sclerosis complex (TSC) 1/2 along the mTORC signaling pathway and inhibit pro-apoptotic proteins or signals.

AKT is known as a survival kinase and mediates cell survival and proliferation by inhibiting pathways including, but not limited to Bcl2 and MDM2, which promotes apoptosis. Studies have shown that the AKT signaling cascade has frequent malfunctions in various cancers, and may be associated with tumor aggressiveness (Nitulescu, G. M. et al., Int J Oncol., 2018; 53(6): 2319-2331). Malfunctions of AKT typically lead to enhanced proliferation, growth, survival, and resistance to apoptosis (Alwhaibi, A. et al., Pharmacol Res., 2019, 145: 104270). Malfunction and mis-regulation of AKT may lead to cancers such as but not limited to breast cancer, gastric carcinoma, glioblastoma, gliosarcomas, head and neck squamous cell carcinoma, ovarian cancer, pancreatic cancer, and prostate cancer.

Additionally, AKT1 has been found to be involved in invasion and migration of cancerous cells (Alwhaibi, A. et al., Pharmacol Res., 2019, 145: 104270). Researchers found that silencing the AKT1 isoform can abrogate specific types of cancer cell migration. However, there have been other studies which have demonstrated that activated AKT1 resulted in less metastatic propensity for lung metastatic lesion cells and breast cancer cells. AKT1 has also been identified as a key protein involved in angiogenesis, lung cancer, and tumorigenesis.

Furthermore, overexpression of AKT has been correlated to resistance to chemotherapeutic agents such as cisplatin, methotrexate, and paclitaxel. Thus, there remains a need to find AKT inhibitors given its role in cell survival and cancer proliferation.

Recently, it has been found that the AKT1 gene mutation E17K can affect cell growth, proliferation, survival, and migration of breast cancer cells, colorectal cancer cells, and ovarian cancer cells (Chen, Y. et al., Front Cell Dev Biol., 2020; 8: 573599). These mutations in the PH structural domain increase the binding of AKT1 to Phosphatidylinositol-3,4,5-triphosphate (PIP3) lipid ligand, which accelerates transfer of AKT from the cytoplasm to the cell membrane through formation of hydrogen bonds. Transfer of AKT into the cell membrane allows it to be further phosphorylated. Once fully activated, AKT can return to the cytoplasm, or go to the nucleus or other intracellular sites, and phosphorylate other substrate proteins to regulate cell function.

The E17K mutation enhances migration of breast cancer cells, and also enhances resistance to chemotherapeutic drugs. However, the E17K mutation can also selectively destroy chemo-resistant tumor-promoting AKT1 quiescent cancer cells, suggesting that the AKT1(E17K) mutation is crucial in the oncogenic/anti-tumor mechanism.

A major pathway that activates PI3K-AKT signaling pathway is somatic cell mutations, with the E17K mutation being the highest frequency of AKT1 mutations. It is nearly exclusively present in AKT1. The AKT1(E17K) is a recurrent somatic cell mutation predominantly in breast cancer, ovarian cancer, meningioma, and Proteus syndrome.

AKT1(E17K) mutations mediate the PI3K-AKT signaling cascade by expanding PIP lipid specificity, which causes conformational changes. This also enhances subcellular localization to accelerate localization of the PH structural domain to the plasma membrane. The E17K mutation increases PIP3 binding specificity by 7-fold and phosphatidylinositol-(4,5)-bisphosphate (PIP2) by 100-fold.

The AKT1(E17K) mutation also causes rapid conformational changes in the AKT1 PH structural domain. The conformational changes to this domain result in a 4.5-fold increase in its membrane localization, which can result in excessive phosphorylation. The AKT1(E17K) mutation can also result in enhanced subcellular localization by increasing the transient expression.

Given the conformational and signaling effects of the AKT1(E17K) mutation, this target may be useful for targeted treatment of cancers.

### Prior Art AKT1 Inhibitors

Most AKT inhibitors targeting the ATP binding site are non-selective against the three isoforms, as well as having poor to no selectivity against other structurally similar kinases. Thus, there remains a need to develop new and novel AKT inhibitors. These ATP targeting inhibitors are classified as aminofurazans, azepane derivatives, isoquinoline-5-sulfonamides, phenylpyrazole derivatives, thiophene carboxamide derivatives, and thiazole carboxamide derivatives.

There are also ATP non-competitive AKT inhibitors which are allosteric modulators which have greater specificity than the ATP targeting inhibitors. Many of these allosteric modulator inhibitors are classified as purine derivatives, thiourea derivatives, alkylphospholipids, sulfonamides, 2,3-diphenylquinoxaline analogs, and indole-3-carbinol derivatives.

### Novel AKT1 Inhibitory Compounds

In one aspect, provided herein is an AKT1 inhibitory compound.

One embodiment provides a compound having the structure of Formula (I), or a pharmaceutically acceptable salt or solvate thereof:

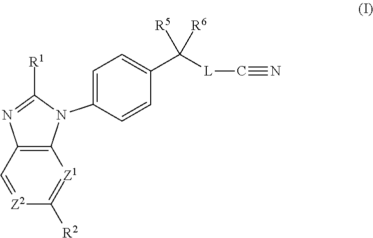

wherein:
Z¹ is N, C—H, or C—R³;
Z² is N, C—H, or C—R⁴;
R¹ is selected from hydrogen, halogen, —CN, optionally substituted C1-C6 alkyl, optionally substituted aryl, or optionally substituted heteroaryl;
R² is selected from hydrogen, halogen, —CN, optionally substituted C1-C6 alkyl, optionally substituted aryl, or optionally substituted heteroaryl;
R³ is selected from halogen, —CN, optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
L is selected from —N(R⁷)—, or a divalent radical selected from:

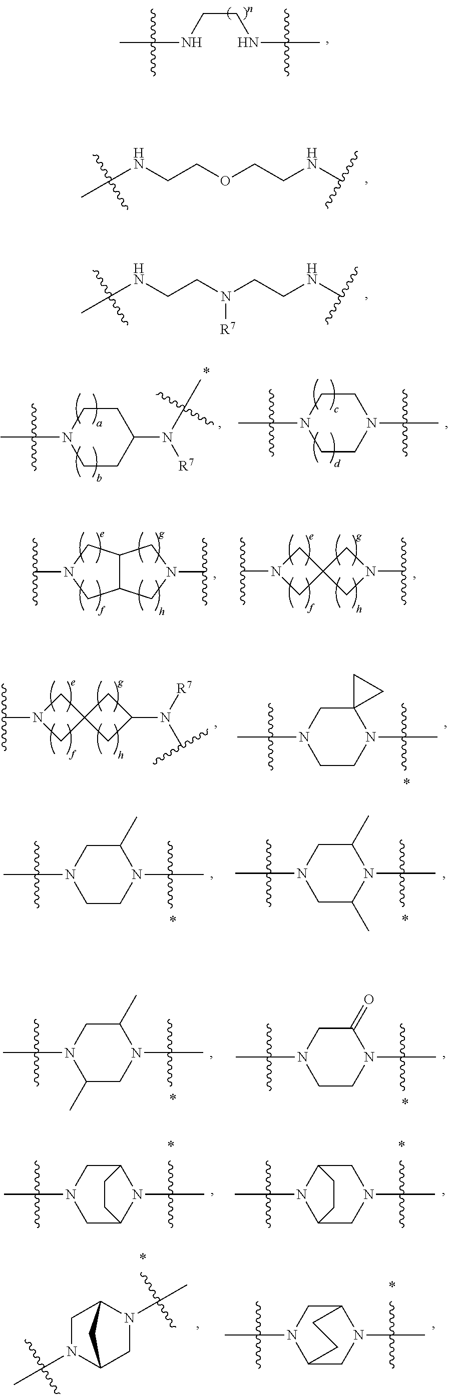

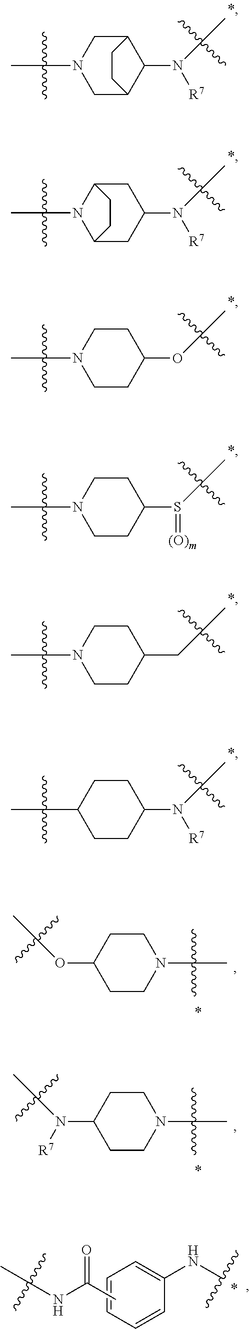

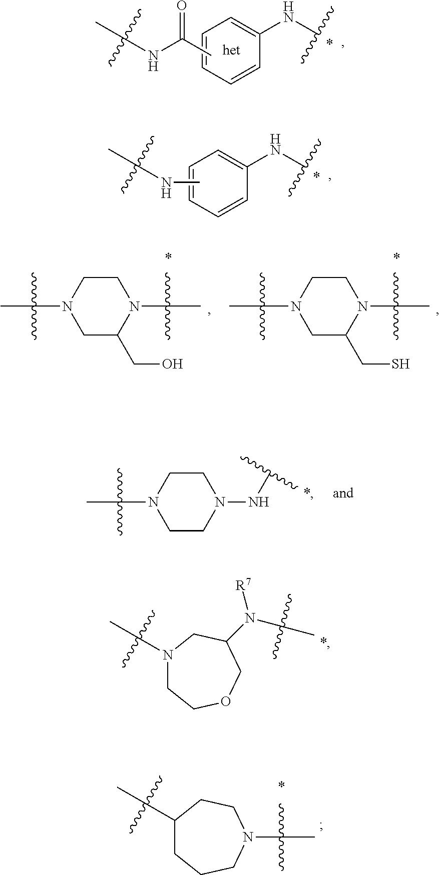

wherein the asterisk (*) indicates the bond to the cyanamide group;
a is 0, 1, 2, 3, or 4;
b is 0, 1, 2, 3, or 4;
c is 1, 2, 3, or 4;
d is 1, 2, 3, or 4;
e is 0, 1, 2, 3, or 4;
f is 0, 1, 2, 3, or 4; provided that e and f are not both 0;
g is 0, 1, 2, 3, or 4; provided that e and g are not both 0;
h is 0, 1, 2, 3, or 4; provided that g and h are not both 0; and provided that f and h are not both 0;
m is 0, 1, or 2;
n is 1, 2, or 3; and
R⁷ is hydrogen, optionally substituted C1-C6 alkyl, optionally substituted C3-C7 cycloalkyl, or optionally substituted heterocyclyl.

Another embodiment provides the compound of Formula (I), or pharmaceutically acceptable salt or solvate thereof, wherein Z¹ is N.

Another embodiment provides the compound of Formula (I), or pharmaceutically acceptable salt or solvate thereof, wherein Z² is C—H.

Another embodiment provides the compound of Formula (I), or pharmaceutically acceptable salt or solvate thereof, wherein Z² is C—R⁴.

Another embodiment provides the compound of Formula (I), or pharmaceutically acceptable salt or solvate thereof, wherein R¹ is optionally substituted heteroaryl. Another embodiment provides the compound of Formula (I), or pharmaceutically acceptable salt or solvate thereof, wherein the optionally substituted heteroaryl is an optionally substituted pyridyl.

Another embodiment provides the compound of Formula (I), or pharmaceutically acceptable salt or solvate thereof, wherein R² is optionally substituted aryl. Another embodiment provides the compound of Formula (I), or pharmaceutically acceptable salt or solvate thereof, wherein the optionally substituted aryl is an optionally substituted phenyl.

Another embodiment provides the compound of Formula (I), or pharmaceutically acceptable salt or solvate thereof, wherein R² is hydrogen.

Another embodiment provides the compound of Formula (I), or pharmaceutically acceptable salt or solvate thereof, wherein Z¹ is N; R² is hydrogen; and R⁴ is an optionally substituted phenyl.

Another embodiment provides the compound of Formula (I), or pharmaceutically acceptable salt or solvate thereof, wherein R³ is optionally substituted C1-C6 alkyl.

Another embodiment provides the compound of Formula (I), or pharmaceutically acceptable salt or solvate thereof, wherein R³ is optionally substituted aryl.

Another embodiment provides the compound of Formula (I), or pharmaceutically acceptable salt or solvate thereof, wherein R⁴ is optionally substituted C1-C6 alkyl.

Another embodiment provides the compound of Formula (I), or pharmaceutically acceptable salt or solvate thereof, wherein R⁴ is optionally substituted aryl.

Another embodiment provides the compound of Formula (I), or pharmaceutically acceptable salt or solvate thereof, wherein R⁵ is hydrogen.

Another embodiment provides the compound of Formula (I), or pharmaceutically acceptable salt or solvate thereof, wherein R⁶ is hydrogen.

Another embodiment provides the compound of Formula (I), or pharmaceutically acceptable salt or solvate thereof, wherein R⁵ and R⁶ together form an oxo.

Another embodiment provides the compound of Formula (I), or pharmaceutically acceptable salt or solvate thereof, wherein R⁶ is optionally substituted C1-C6 alkyl.

Another embodiment provides the compound of Formula (I), or pharmaceutically acceptable salt or solvate thereof, wherein R⁵ and R⁶ join together to form a carbocycle or heterocycle.

Another embodiment provides the compound of Formula (I), or pharmaceutically acceptable salt or solvate thereof, wherein L is —N(R⁷)—.

Another embodiment provides the compound of Formula (I), or pharmaceutically acceptable salt or solvate thereof, wherein L is selected from:

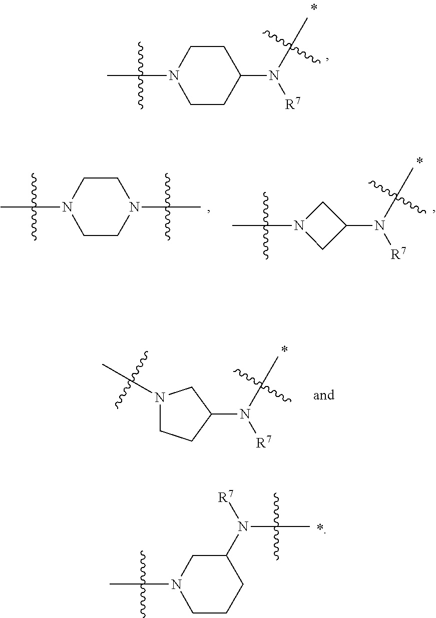

Another embodiment provides the compound of Formula (I), or pharmaceutically acceptable salt or solvate thereof, wherein L is selected from:

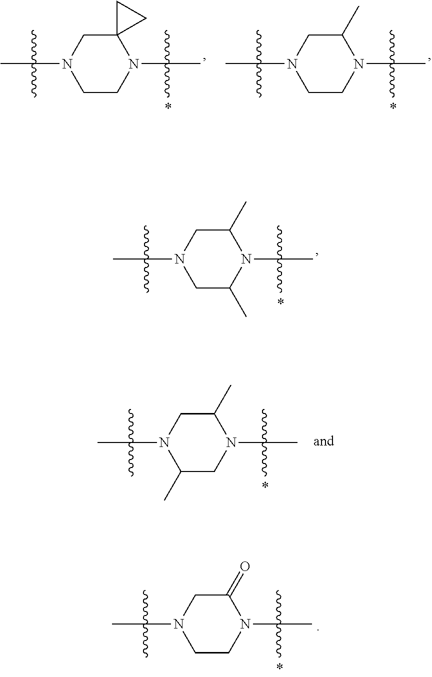

Another embodiment provides the compound of Formula (I), or pharmaceutically acceptable salt or solvate thereof, wherein L is selected from:

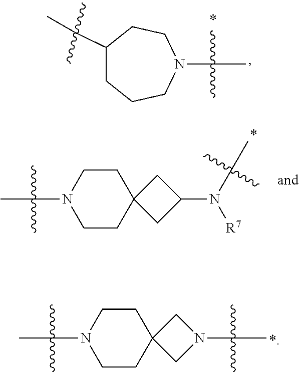

Another embodiment provides the compound of Formula (I), or pharmaceutically acceptable salt or solvate thereof, wherein L is selected from:

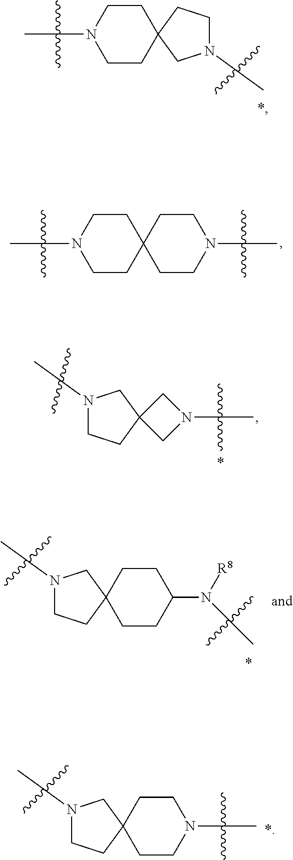

Another embodiment provides the compound of Formula (I), or pharmaceutically acceptable salt or solvate thereof, wherein L is selected from:

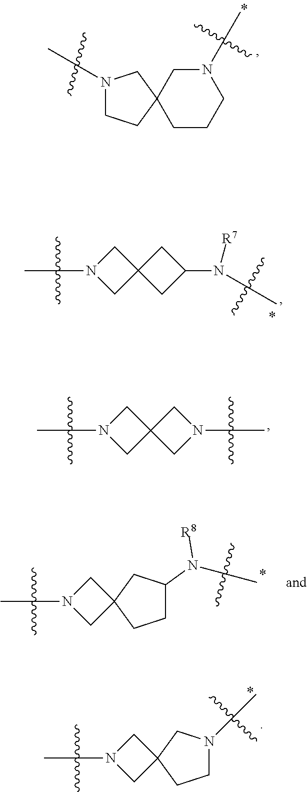

Another embodiment provides the compound of Formula (I), or pharmaceutically acceptable salt or solvate thereof, wherein L is selected from:

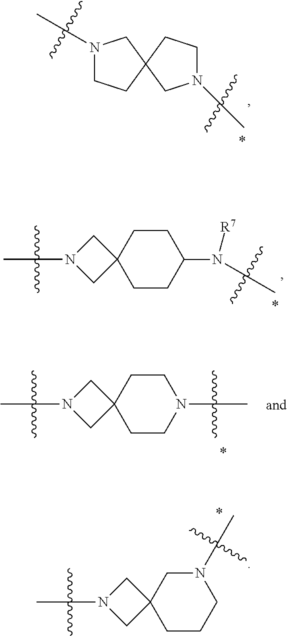

Another embodiment provides the compound of Formula (I), or pharmaceutically acceptable salt or solvate thereof, wherein L is

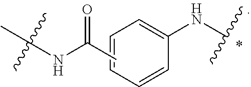

Another embodiment provides the compound of Formula (I), or pharmaceutically acceptable salt or solvate thereof, wherein L is

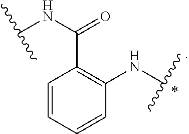 [SMILES: *NC(=O)c1ccccc1N*]

Another embodiment provides the compound of Formula (I), or pharmaceutically acceptable salt or solvate thereof, wherein L is

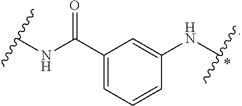 [SMILES: *NC(=O)c1cccc(N*)c1]

Another embodiment provides the compound of Formula (I), or pharmaceutically acceptable salt or solvate thereof, wherein L is

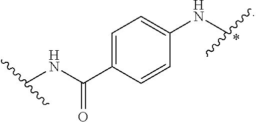 [SMILES: *NC(=O)c1ccc(N*)cc1]

Another embodiment provides the compound of Formula (I), or pharmaceutically acceptable salt or solvate thereof, wherein L is

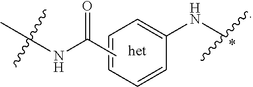

Another embodiment provides the compound of Formula (I), or pharmaceutically acceptable salt or solvate thereof, wherein L is

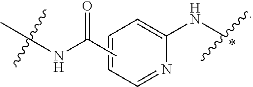

Another embodiment provides the compound of Formula (I), or pharmaceutically acceptable salt or solvate thereof, wherein L is

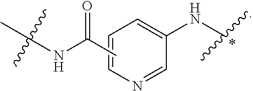

Another embodiment provides the compound of Formula (I), or pharmaceutically acceptable salt or solvate thereof, wherein L is

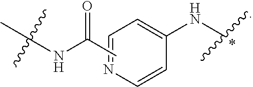

Another embodiment provides the compound of Formula (I), or pharmaceutically acceptable salt or solvate thereof, wherein L is

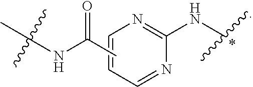

Another embodiment provides the compound of Formula (I), or pharmaceutically acceptable salt or solvate thereof, wherein L is

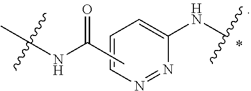

Another embodiment provides the compound of Formula (I), or pharmaceutically acceptable salt or solvate thereof, wherein L is

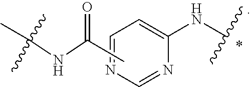

Another embodiment provides the compound of Formula (I), or pharmaceutically acceptable salt or solvate thereof, wherein L is

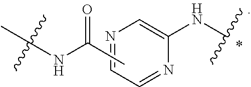

Another embodiment provides the compound of Formula (I), or pharmaceutically acceptable salt or solvate thereof, wherein L is

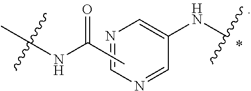

Another embodiment provides the compound of Formula (I), or pharmaceutically acceptable salt or solvate thereof, wherein L is

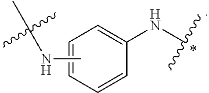

Another embodiment provides the compound of Formula (I), or pharmaceutically acceptable salt or solvate thereof, wherein L is

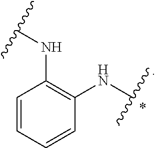 [SMILES: *Nc1ccccc1N*]

Another embodiment provides the compound of Formula (I), or pharmaceutically acceptable salt or solvate thereof, wherein L is

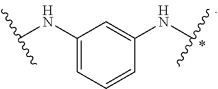 [SMILES: *Nc1cccc(N*)c1]

Another embodiment provides the compound of Formula (I), or pharmaceutically acceptable salt or solvate thereof, wherein L is

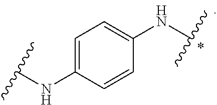 [SMILES: *Nc1ccc(N*)cc1]

Another embodiment provides the compound of Formula (I), or pharmaceutically acceptable salt or solvate thereof, wherein R⁷ is hydrogen or optionally substituted C1-C6 alkyl.

One embodiment provides an AKT1 inhibitory compound, or a pharmaceutically acceptable salt or solvate thereof, having a structure presented in Table 1.

TABLE 1

| Example | | |
|---|---|---|
| Number | Structure | Name |
| 1 | 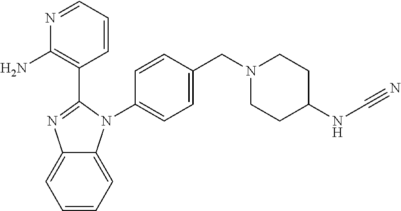 [SMILES: N#CNC1CCN(Cc2ccc(-n3c(-c4cccnc4N)nc4ccccc43)cc2)CC1] | N-(1-(4-(2-(2-aminopyridin-3-yl)- 3H-imidazo[4,5-b]pyridin-3- yl)benzyl)piperidin-4- yl)cyanamide |
| 2 | 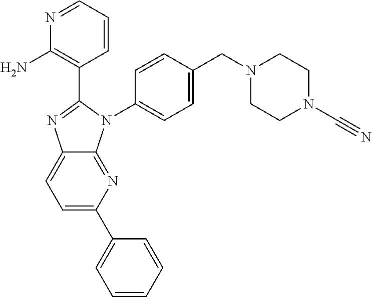 [SMILES: N#CN1CCN(Cc2ccc(-n3c(-c4cccnc4N)nc4ccc(-c5ccccc5)nc43)cc2)CC1] | 4-(4-(2-(2-aminopyridin-3-yl)-5- phenyl-3H-imidazo[4,5-b]pyridin- 3-yl)benzyl)piperazine-1- carbonitrile |
| 3 | 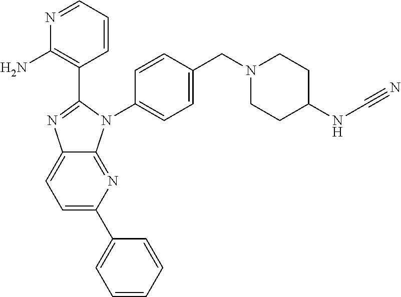 [SMILES: N#CNC1CCN(Cc2ccc(-n3c(-c4cccnc4N)nc4ccc(-c5ccccc5)nc43)cc2)CC1] | N-(1-(4-(2-(2-aminopyridin-3-yl)- 5-phenyl-3H-imidazo[4,5- b]pyridin-3-yl)benzyl)piperidin-4- yl)cyanamide |
| 4 | 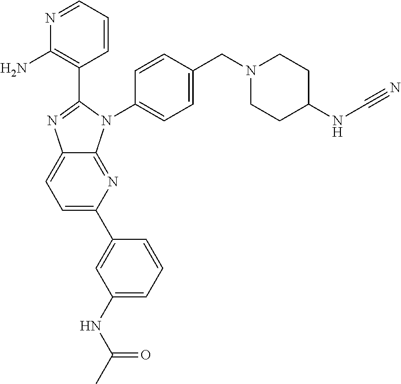 [SMILES: CC(=O)Nc1cccc(-c2ccc3nc(-c4cccnc4N)n(-c4ccc(CN5CCC(NC#N)CC5)cc4)c3n2)c1] | N-(3-(2-(2-aminopyridin-3-y1)-3- (4-((4-cyanamidopiperidin-1- yl)methyl)phenyl)-3H- imidazo[4,5-b]pyridin-5- yl)phenyl)acetamide |
| 5 | 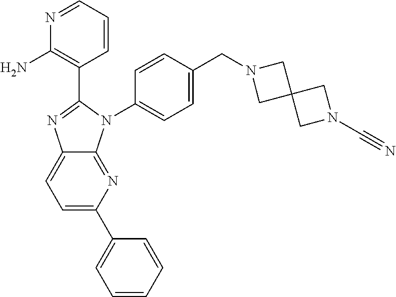 [SMILES: N#CN1CC2(C1)CN(Cc1ccc(-n3c(-c4cccnc4N)nc4ccc(-c5ccccc5)nc43)cc1)C2] | 6-(4-(2-(2-aminopyridin-3-yl)-5- phenyl-3H-imidazo[4,5-b]pyridin- 3-yl)benzyl)-2,6- diazaspiro[3.3 ]heptane-2- carbonitrile |
| 6 | 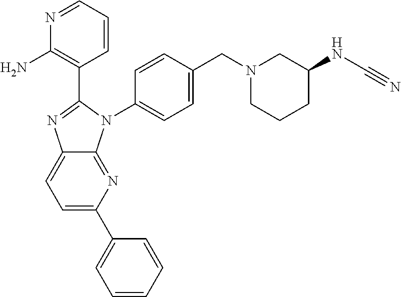 [SMILES: N#CN[C@H]1CCCN(Cc2ccc(-n3c(-c4cccnc4N)nc4ccc(-c5ccccc5)nc43)cc2)C1] | (S)-N-(1-(4-(2-(2-aminopyridin-3- yl)-5-phenyl-3H-imidazo[4,5- b]pyridin-3-yl)benzyl)piperidin-3- yl)cyanamide |
| 7 | 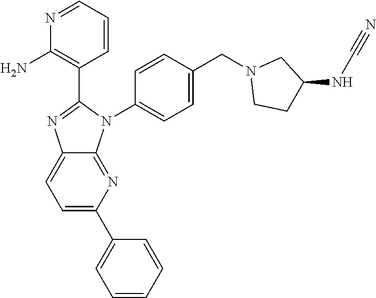 [SMILES: N#CN[C@H]1CCN(Cc2ccc(-n3c(-c4cccnc4N)nc4ccc(-c5ccccc5)nc43)cc2)C1] | (S)-N-(1-(4-(2-(2-aminopyridin-3- yl)-5-phenyl-3H-imidazo[4,5- b]pyridin-3-yl)benzyl)pyrrolidin- 3-yl)cyanamide |
| 8 | 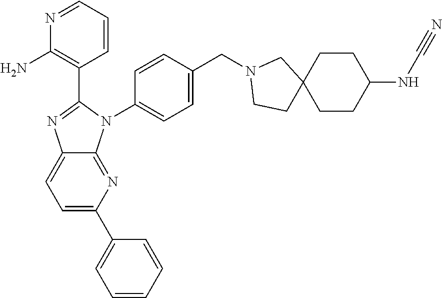 [SMILES: N#CNC1CCC2(CC1)CCN(Cc1ccc(-n3c(-c4cccnc4N)nc4ccc(-c5ccccc5)nc43)cc1)C2] | N-(2-(4-(2-(2-aminopyridin-3-yl)- 5-phenyl-3H-imidazo[4,5- b]pyridin-3-yl)benzyl)-2- azaspiro[4.5]decan-8- yl)cyanamide |
| 9 | 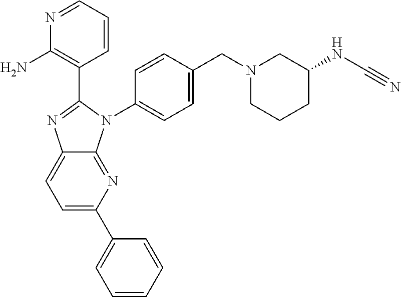 [SMILES: N#CN[C@@H]1CCCN(Cc2ccc(-n3c(-c4cccnc4N)nc4ccc(-c5ccccc5)nc43)cc2)C1] | (R)-N-(1-(4-(2-(2-aminopyridin-3- yl)-5-phenyl-3H-imidazo[4,5- b]pyridin-3-yl)benzyl)piperidin-3- yl)cyanamide |
| 10 | 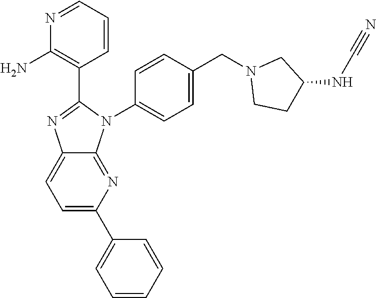 [SMILES: N#CN[C@@H]1CCN(Cc2ccc(-n3c(-c4cccnc4N)nc4ccc(-c5ccccc5)nc43)cc2)C1] | (R)-N-(1-(4-(2-(2-aminopyridin-3- yl)-5-phenyl-3H-imidazo[4,5- b]pyridin-3-yl)benzyl)pyrrolidin- 3-yl)cyanamide |
| 11 | 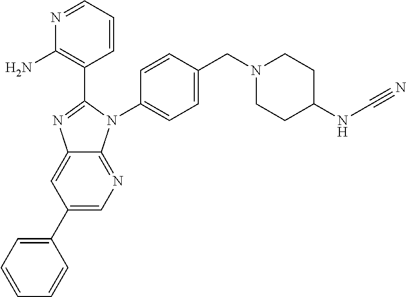 [SMILES: N#CNC1CCN(Cc2ccc(-n3c(-c4cccnc4N)nc4cc(-c5ccccc5)cnc43)cc2)CC1] | N-(1-(4-(2-(2-aminopyridin-3-yl)- 6-phenyl-3H-imidazo[4,5- b]pyridin-3-yl)benzyl)piperidin-4- yl)cyanamide |
| 12 | 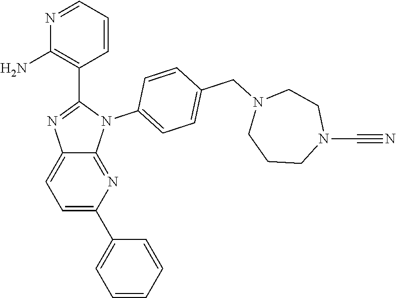 [SMILES: N#CN1CCCN(Cc2ccc(-n3c(-c4cccnc4N)nc4ccc(-c5ccccc5)nc43)cc2)CC1] | 4-(4-(2-(2-aminopyridin-3-yl)-5- phenyl-3H-imidazo[4,5-b]pyridin- 3-yl)benzyl)-1,4-diazepane-1- carbonitrile |
| 13 | 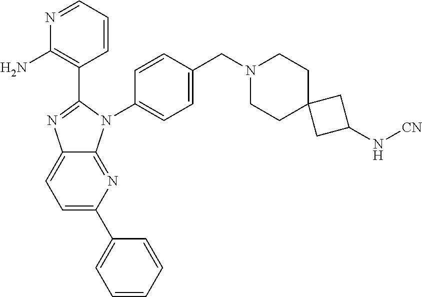 [SMILES: N#CNC1CC2(CCN(Cc3ccc(-n4c(-c5cccnc5N)nc5ccc(-c6ccccc6)nc54)cc3)CC2)C1] | N-(7-(4-(2-(2-aminopyridin-3-yl)- 5-phenyl-3H-imidazo[4,5- b]pyridin-3-yl)benzyl)-7- azaspiro[3.5]nonan-2- yl)cyanamide |
| 14 | 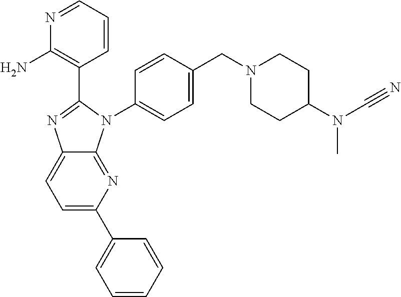 [SMILES: CN(C#N)C1CCN(Cc2ccc(-n3c(-c4cccnc4N)nc4ccc(-c5ccccc5)nc43)cc2)CC1] | N-(1-(4-(2-(2-aminopyridin-3-yl)- 5-phenyl-3H-imidazo[4,5- b]pyridin-3-yl)benzyl)piperidin-4- yl)-N-methylcyanamide |
| 15 | 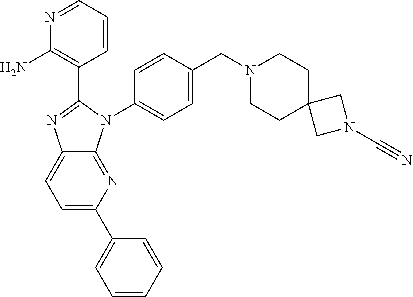 [SMILES: N#CN1CC2(CCN(Cc3ccc(-n4c(-c5cccnc5N)nc5ccc(-c6ccccc6)nc54)cc3)CC2)C1] | 7-(4-(2-(2-aminopyridin-3-yl)-5- phenyl-3H-imidazo[4,5-b]pyridin- 3-yl)benzyl)-2,7- diazaspiro[3.5]nonane-2- carbonitrile |
| 16 | 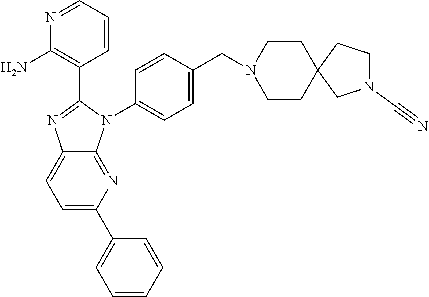 [SMILES: N#CN1CCC2(CCN(Cc3ccc(-n4c(-c5cccnc5N)nc5ccc(-c6ccccc6)nc54)cc3)CC2)C1] | 8-(4-(2-(2-aminopyridin-3-y1)-5- phenyl-3H-imidazo[4,5-b]pyridin- 3-yl)benzyl)-2,8- diazaspiro[4.5]decane-2- carbonitrile |
| 17 | 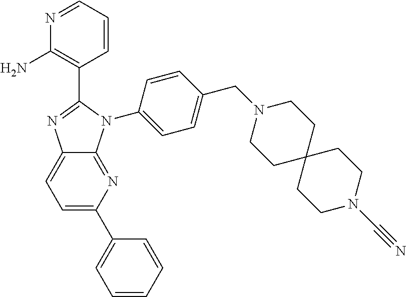 [SMILES: N#CN1CCC2(CC1)CCN(Cc1ccc(-n3c(-c4cccnc4N)nc4ccc(-c5ccccc5)nc43)cc1)CC2] | 9-(4-(2-(2-aminopyridin-3-y1)-5- phenyl-3H-imidazo[4,5-b]pyridin- 3-yl)benzyl)-3,9- diazaspiro[5.5]undecane-3- carbonitrile |
| 18 | 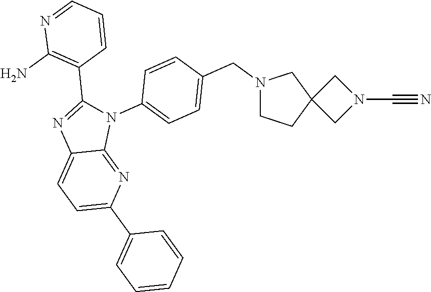 [SMILES: N#CN1CC2(CCN(Cc3ccc(-n4c(-c5cccnc5N)nc5ccc(-c6ccccc6)nc54)cc3)C2)C1] | 6-(4-(2-(2-aminopyridin-3-yl)-5- phenyl-3H-imidazo[4,5-b]pyridin- 3-yl)benzyl)-2,6- diazaspiro[3.4]octane-2- carbonitrile |
| 19 | 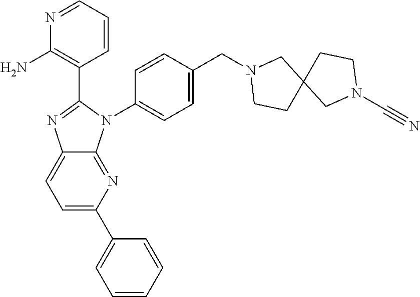 [SMILES: N#CN1CCC2(CCN(Cc3ccc(-n4c(-c5cccnc5N)nc5ccc(-c6ccccc6)nc54)cc3)C2)C1] | 7-(4-(2-(2-aminopyridin-3-y1)-5- phenyl-3H-imidazo[4,5-b]pyridin- 3-yl)benzyl)-2,7- diazaspiro[4.4]nonane-2- carbonitrile |
| 20 | 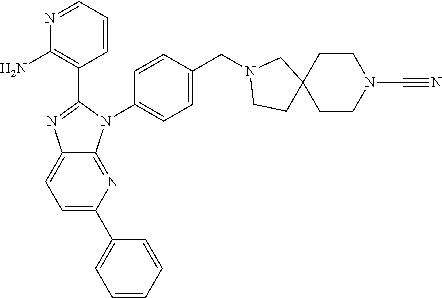 [SMILES: N#CN1CCC2(CC1)CCN(Cc1ccc(-n3c(-c4cccnc4N)nc4ccc(-c5ccccc5)nc43)cc1)C2] | 2-(4-(2-(2-aminopyridin-3-yl)-5- phenyl-3H-imidazo[4,5-b]pyridin- 3-yl)benzyl)-2,8- diazaspiro[4.5]decane-8- carbonitrile |
| 21 | 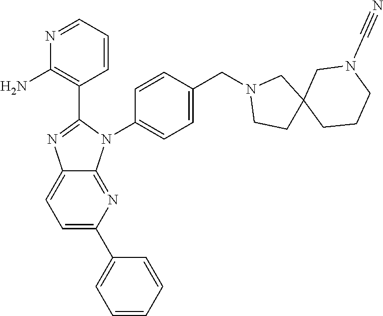 [SMILES: N#CN1CCCC2(CCN(Cc3ccc(-n4c(-c5cccnc5N)nc5ccc(-c6ccccc6)nc54)cc3)C2)C1] | 2-(4-(2-(2-aminopyridin-3-yl)-5- phenyl-3H-imidazo[4,5-b]pyridin- 3-yl)benzyl)-2,7- diazaspiro[4.5]decane-7- carbonitrile |
| 22 | 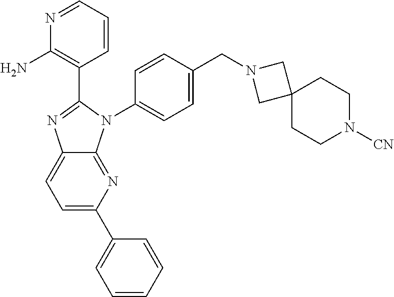 [SMILES: N#CN1CCC2(CC1)CN(Cc1ccc(-n3c(-c4cccnc4N)nc4ccc(-c5ccccc5)nc43)cc1)C2] | 2-(4-(2-(2-aminopyridin-3-yl)-5- phenyl-3H-imidazo[4,5-b]pyridin- 3-yl)benzyl)-2,7- diazaspiro[3.5]nonane-7- carbonitrile |
| 23 | 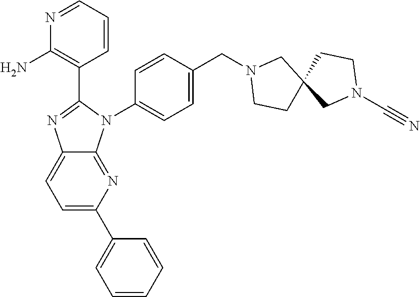 [SMILES: N#CN1CC[C@@]2(CCN(Cc3ccc(-n4c(-c5cccnc5N)nc5ccc(-c6ccccc6)nc54)cc3)C2)C1] | (R)-7-(4-(2-(2-aminopyridin-3-yl)- 5-phenyl-3H-imidazo[4,5- b]pyridin-3-yl)benzyl)-2,7- diazaspiro[4.4]nonane-2- carbonitrile |
| 24 | 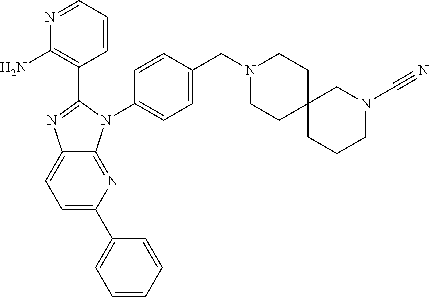 [SMILES: N#CN1CCCC2(CCN(Cc3ccc(-n4c(-c5cccnc5N)nc5ccc(-c6ccccc6)nc54)cc3)CC2)C1] | 9-(4-(2-(2-aminopyridin-3-yl)-5- phenyl-3H-imidazo[4,5-b]pyridin- 3-yl)benzyl)-2,9- diazaspiro[5.5]undecane-2- carbonitrile |
| 25 | 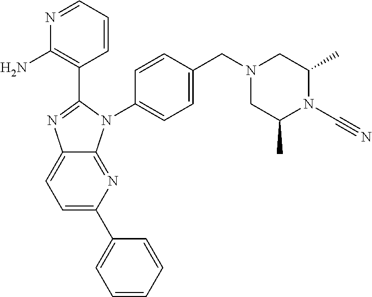 [SMILES: C[C@H]1CN(Cc2ccc(-n3c(-c4cccnc4N)nc4ccc(-c5ccccc5)nc43)cc2)C[C@H](C)N1C#N] | (2S,6S)-4-(4-(2-(2-aminopyridin- 3-yl)-5-phenyl-3H-imidazo[4,5- b]pyridin-3-yl)benzyl)-2,6- dimethylpiperazine-1-carbonitrile |
| 26 | 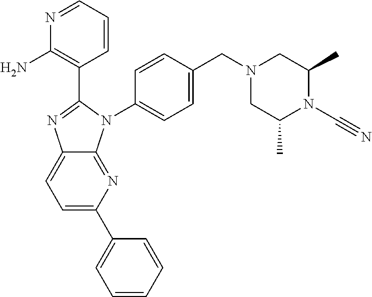 [SMILES: C[C@@H]1CN(Cc2ccc(-n3c(-c4cccnc4N)nc4ccc(-c5ccccc5)nc43)cc2)C[C@@H](C)N1C#N] | (2R,6R)-4-(4-(2-(2-aminopyridin- 3-y1)-5-phenyl-3H-imidazo[4,5- b]pyridin-3-yl)benzyl)-2,6- dimethylpiperazine-1-carbonitrile |
| 27 | 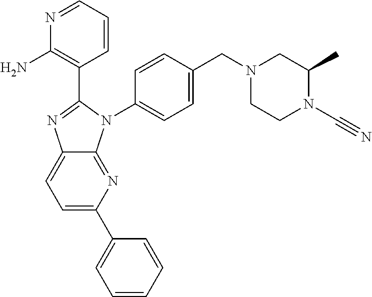 [SMILES: C[C@@H]1CN(Cc2ccc(-n3c(-c4cccnc4N)nc4ccc(-c5ccccc5)nc43)cc2)CCN1C#N] | (R)-4-(4-(2-(2-aminopyridin-3-yl)- 5-phenyl-3H-imidazo[4,5- b]pyridin-3-yl)benzyl)-2- methylpiperazine-1-carbonitrile |
| 28 | 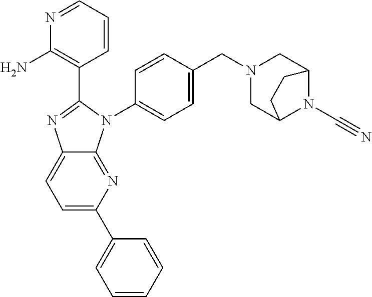 [SMILES: N#CN1C2CCC1CN(Cc1ccc(-n3c(-c4cccnc4N)nc4ccc(-c5ccccc5)nc43)cc1)C2] | 3-(4-(2-(2-aminopyridin-3-yl)-5- phenyl-3H-imidazo[4,5-b]pyridin- 3-yl)benzyl)-3,8- diazabicyclo[3.2.1 ]octane-8- carbonitrile |
| 29 | 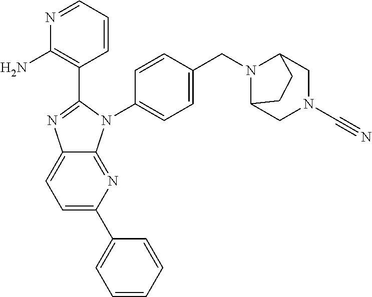 [SMILES: N#CN1CC2CCC(C1)N2Cc1ccc(-n2c(-c3cccnc3N)nc3ccc(-c4ccccc4)nc32)cc1] | 8-(4-(2-(2-aminopyridin-3-yl)-5- phenyl-3H-imidazo[4,5-b]pyridin- 3-yl)benzyl)-3,8- diazabicyclo[3.2.1 ]octane-3- carbonitrile |
| 30 | 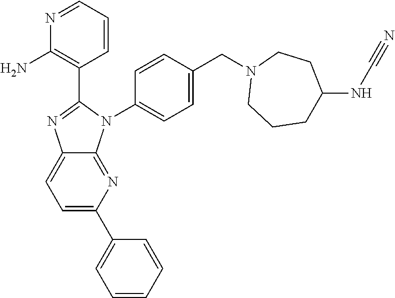 [SMILES: N#CNC1CCCN(Cc2ccc(-n3c(-c4cccnc4N)nc4ccc(-c5ccccc5)nc43)cc2)CC1] | N-(1-(4-(2-(2-aminopyridin-3-yl)- 5-phenyl-3H-imidazo[4,5- b]pyridin-3-yl)benzyl)azepan-4- yl)cyanamide |
| 31 | 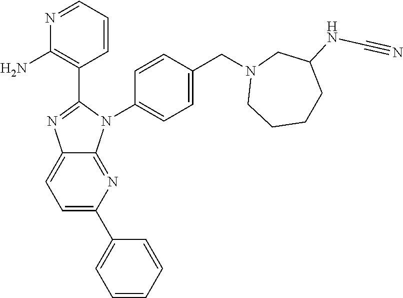 [SMILES: N#CNC1CCCCN(Cc2ccc(-n3c(-c4cccnc4N)nc4ccc(-c5ccccc5)nc43)cc2)C1] | N-(1-(4-(2-(2-Aminopyridin-3-yl)- 5-phenyl-3H-imidazo[4,5- b]pyridin-3-yl)benzyl)azepan-3- yl)cyanamide |
| 32 | 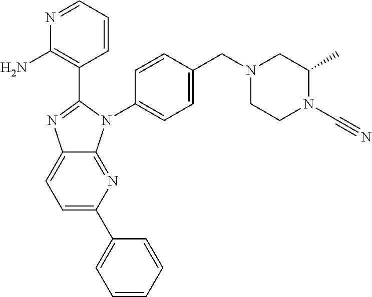 [SMILES: C[C@H]1CN(Cc2ccc(-n3c(-c4cccnc4N)nc4ccc(-c5ccccc5)nc43)cc2)CCN1C#N] | (S)-4-(4-(2-(2-aminopyridin-3-yl)- 5-phenyl-3H-imidazo[4,5- b]pyridin-3-yl)benzyl)-2- methylpiperazine-1-carbonitrile |
| 33 | 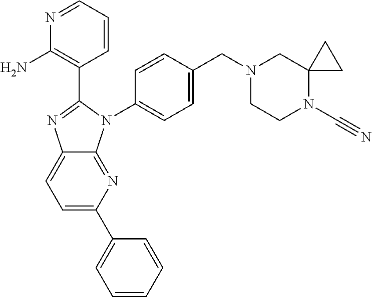 [SMILES: N#CN1CCN(Cc2ccc(-n3c(-c4cccnc4N)nc4ccc(-c5ccccc5)nc43)cc2)CC12CC2] | 7-(4-(2-(2-aminopyridin-3-yl)-5- phenyl-3H-imidazo[4,5-b]pyridin- 3-yl)benzyl)-4,7- diazaspiro[2.5]octane-4- carbonitrile |
| 34 | 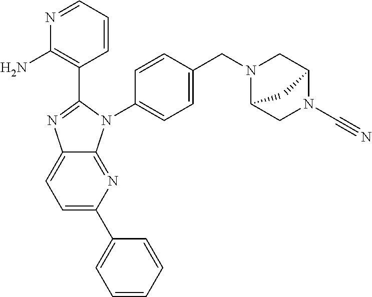 [SMILES: N#CN1C[C@@H]2C[C@H]1CN2Cc1ccc(-n2c(-c3cccnc3N)nc3ccc(-c4ccccc4)nc32)cc1] | (1S,4S)-5-(4-(2-(2-aminopyridin- 3-yl)-5-phenyl-3H-imidazo[4,5- b]pyridin-3-yl)benzyl)-2,5- diazabicyclo[2.2.1 ]heptane-2- carbonitrile |
| 35 | 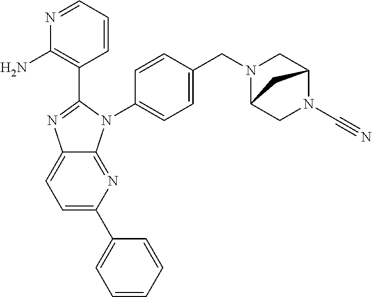 [SMILES: N#CN1C[C@H]2C[C@@H]1CN2Cc1ccc(-n2c(-c3cccnc3N)nc3ccc(-c4ccccc4)nc32)cc1] | (1R,4R)-5-(4-(2-(2-aminopyridin- 3-y1)-5-phenyl-3H-imidazo[4,5- b]pyridin-3-yl)benzyl)-2,5- diazabicyclo[2.2.1 ]heptane-2- carbonitrile |
| 36 | 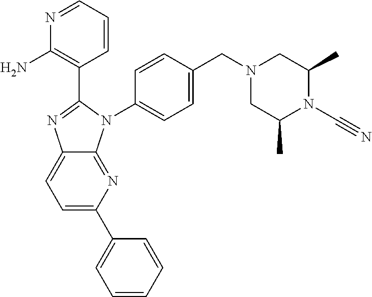 [SMILES: C[C@@H]1CN(Cc2ccc(-n3c(-c4cccnc4N)nc4ccc(-c5ccccc5)nc43)cc2)C[C@H](C)N1C#N] | (2S,6R)-4-(4-(2-(2-aminopyridin- 3-y1)-5-phenyl-3H-imidazo[4,5- b]pyridin-3-yl)benzyl)-2,6- dimethylpiperazine-1-carbonitrile |
| 37 | 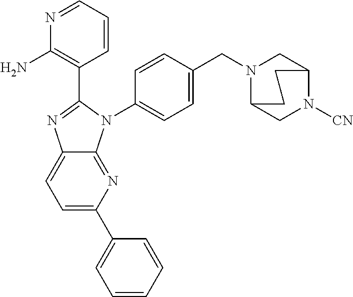 [SMILES: N#CN1CC2CCC1CN2Cc1ccc(-n2c(-c3cccnc3N)nc3ccc(-c4ccccc4)nc32)cc1] | 5-(4-(2-(2-aminopyridin-3-yl)-5- phenyl-3H-imidazo[4,5-b]pyridin- 3-yl)benzyl)-2,5- diazabicyclo[2.2.2]octane-2- carbonitrile |
| 38 | 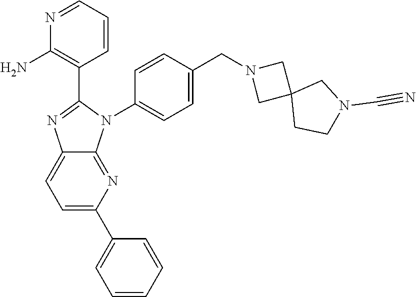 [SMILES: N#CN1CCC2(C1)CN(Cc1ccc(-n3c(-c4cccnc4N)nc4ccc(-c5ccccc5)nc43)cc1)C2] | 2-(4-(2-(2-aminopyridin-3-yl)-5- phenyl-3H-imidazo[4,5-b]pyridin- 3-yl)benzyl)-2,6- diazaspiro[3.4]octane-6- carbonitrile |
| 39 | 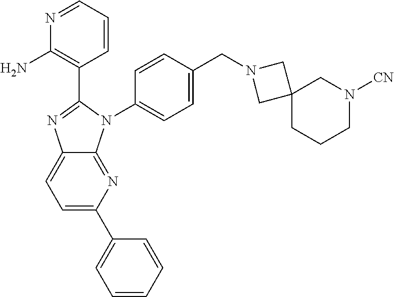 [SMILES: N#CN1CCCC2(C1)CN(Cc1ccc(-n3c(-c4cccnc4N)nc4ccc(-c5ccccc5)nc43)cc1)C2] | 2-(4-(2-(2-aminopyridin-3-y1)-5- phenyl-3H-imidazo[4,5-b]pyridin- 3-yl)benzyl)-2,6- diazaspiro[3.5]nonane-6- carbonitrile |
| 40 | 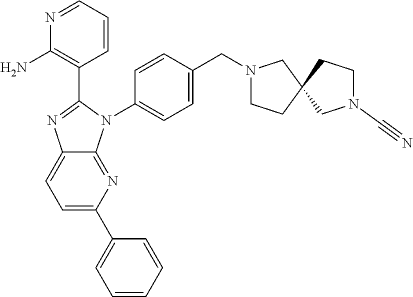 [SMILES: N#CN1CC[C@]2(CCN(Cc3ccc(-n4c(-c5cccnc5N)nc5ccc(-c6ccccc6)nc54)cc3)C2)C1] | (S)-7-(4-(2-(2-aminopyridin-3-yl)- 5-phenyl-3H-imidazo[4,5- b]pyridin-3-yl)benzyl)-2,7- diazaspiro[4.4]nonane-2- carbonitrile |
| 41 | 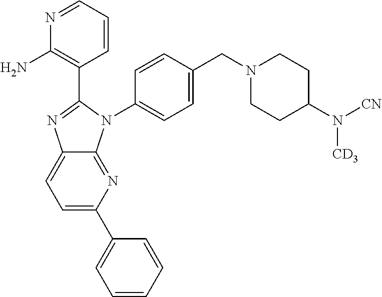 [SMILES: [2H]C([2H])([2H])N(C#N)C1CCN(Cc2ccc(-n3c(-c4cccnc4N)nc4ccc(-c5ccccc5)nc43)cc2)CC1] | N-(1-(4-(2-(2-aminopyridin-3-yl)- 5-phenyl-3H-imidazo[4,5- b]pyridin-3-yl)benzyl)piperidin-4- yl)-N-(methyl-d3)cyanamide |
| 42 | 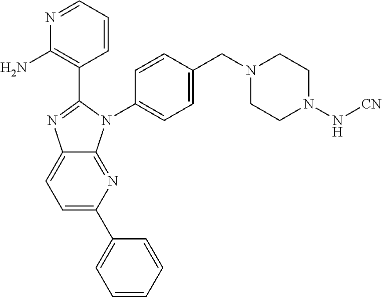 [SMILES: N#CNN1CCN(Cc2ccc(-n3c(-c4cccnc4N)nc4ccc(-c5ccccc5)nc43)cc2)CC1] | N-(4-(4-(2-(2-aminopyridin-3-yl)- 5-phenyl-3H-imidazo[4,5- b]pyridin-3-yl)benzyl)piperazin-1- yl)cyanamide |
| 43 | 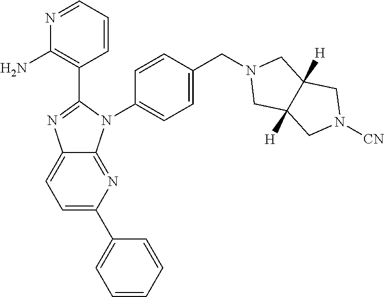 [SMILES: N#CN1C[C@@H]2CN(Cc3ccc(-n4c(-c5cccnc5N)nc5ccc(-c6ccccc6)nc54)cc3)C[C@@H]2C1] | (3aR,6aS)-5-(4-(2-(2- aminopyridin-3-y1)-5-phenyl-3H- imidazo[4,5-b]pyridin-3- yl)benzyl)hexahydropyrrolo[3,4- c]pyrrole-2(1H)-carbonitrile |

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-λ, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the AKT1 inhibitory compound described herein is administered as a pure chemical. In other embodiments, the AKT1 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one AKT1 inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the AKT1 inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.10%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1, or a pharmaceutically acceptable salt or solvate thereof.

One embodiment provides a method of preparing a pharmaceutical composition comprising mixing a compound of Table 1, or a pharmaceutically acceptable salt or solvate thereof, and a pharmaceutically acceptable carrier.

In certain embodiments, the AKT1 inhibitory compound as described by Table 1, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the AKT1 inhibitory compound as described by Formula (I) or Table 1, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one AKT1 inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a pharmaceutical composition comprising a compound of Formula (I), or a pharmaceutically acceptable salt or solvate thereof, and a pharmaceutically acceptable excipient for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), or a pharmaceutically acceptable salt or solvate thereof. In some embodiments is provided a method of treating cancer, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Table 1, or a pharmaceutically acceptable salt or solvate thereof, for use in a method of treatment of the human or animal body.

One embodiment provides a compound of Table 1, or a pharmaceutically acceptable salt or solvate thereof, for use in a method of treatment of cancer or neoplastic disease.

One embodiment provides a pharmaceutical composition comprising a compound of Table 1, or a pharmaceutically acceptable salt or solvate thereof, and a pharmaceutically acceptable excipient for use in a method of treatment of cancer or neoplastic disease.

One embodiment provides a use of a compound of Table 1, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1, or a pharmaceutically acceptable salt or solvate thereof. In some embodiments is provided a method of treating cancer, in a patient in need thereof, comprising administering to the patient a pharmaceutical composition comprising a compound of Table 1, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting an AKT1 enzyme comprising contacting the AKT1 enzyme with a compound of Formula (I), or Table 1. Another embodiment provides the method of inhibiting an AKT1 enzyme, wherein the AKT1 enzyme is contacted in an in vivo setting. Another embodiment provides the method of inhibiting an AKT1 enzyme, wherein the AKT1 enzyme is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the AKT1 inhibitory compounds disclosed herein are synthesized according to the following examples. As used below, and throughout the description of the invention, the following abbreviations, unless otherwise indicated, shall be understood to have the following meanings:
ACN acetonitrile
° C. degrees Celsius
δ_{H} chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP triphenylphosphine

### Experimental Procedures

### Intermediate 1: 3-(3-(4-((4-Aminopiperidin-1-yl)methyl)phenyl)-3H-imidazo[4,5-b]pyridin-2-yl)pyridin-2-amine

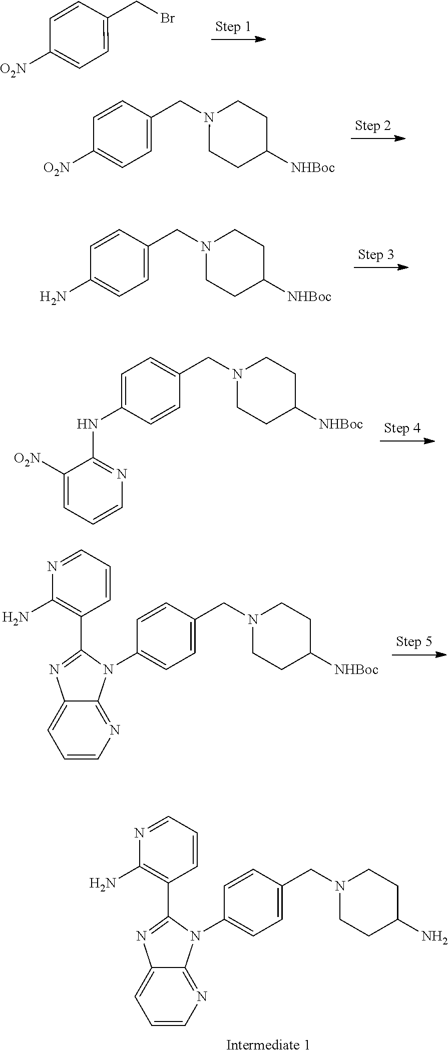

### Step 1: tert-Butyl (1-(4-nitrobenzyl) piperidin-4-yl) carbamate

To a solution of 1-(bromomethyl)-4-nitro-benzene (108 g, 499 mmol) in ACN (1.5 L) were added K₂CO₃ (149 g, 1.1 mol) and tert-butyl N-(4-piperidyl)carbamate (100 g, 499 mmol). The mixture was stirred at 25° C. for 16 hr. The reaction mixture was filtered. The filtrate was concentrated under reduced pressure to give tert-butyl (1-(4-nitrobenzyl) piperidin-4-yl)carbamate (167 g, crude) as a yellow solid, which was used in the next step without further purification. MS: m/z=335.9 [M+H]⁺. ¹H NMR (400 MHz, Chloroform-d) δ 8.16 (d, J=8.8 Hz, 2H), 7.49 (d, J=8.8 Hz, 2H), 4.44 (br s, 1H), 3.56 (s, 2H), 3.52-3.42 (m, 1H), 2.77-2.74 (m, 2H), 2.16-2.10 (m, 2H), 1.93-1.90 (m, 2H), 1.43 (s, 9H), 1.42-1.36 (m, 2H).

### Step 2: tert-Butyl (1-(4-aminobenzyl) piperidin-4-yl) carbamate

To a solution of tert-butyl (1-(4-nitrobenzyl) piperidin-4-yl)carbamate (109 g, 325 mmol) in EtOH (500 mL) and H₂O (150 mL) were added Fe (91 g, 1.6 mol) and NH₄Cl (174 g, 3.3 mol). The mixture was stirred at 85° C. for 2 hr. The reaction mixture was filtered. The filtrate was concentrated under reduced pressure to remove most of the EtOH. The residue was diluted with H₂O (500 mL) and extracted with CH₂Cl₂(500 mL×2). The combined organic layers were washed with brine (500 mL), dried over Na₂SO₄, filtered, and concentrated under reduced pressure to give tert-butyl (1-(4-aminobenzyl) piperidin-4-yl) carbamate (80 g crude) as a yellow solid. MS: m/z=306.2 [M+H]⁺. ¹H NMR (400 MHz, Chloroform-d) δ 7.14 (d, J=8.0 Hz, 2H), 6.64 (d, J=8.0 Hz, 2H), 4.51 (br d, J=6.0 Hz, 1H), 3.80-3.59 (m, 2H), 3.55 (s, 2H), 3.51-3.39 (m, 1H), 2.95-2.93 (m, 2H), 2.25-2.20 (m, 2H), 1.96-1.93 (m, 2H), 1.72-1.54 (m, 2H), 1.42 (s, 9H).

### Step 3: tert-Butyl (1-(4-((3-nitropyridin-2-yl)amino)benzyl)piperidin-4-yl)carbamate

To a solution of tert-butyl (1-(4-aminobenzyl) piperidin-4-yl) carbamate (30 g, 98.2 mmol) in DMSO (500 mL) were added DIEA (38.1 g, 295 mmol) and 2-chloro-3-nitro-pyridine (18.7 g, 118 mmol). The mixture was stirred at 100° C. for 16 hr. The reaction mixture was quenched with H₂O (500 mL) at 20° C. and extracted with EtOAc (300 mL×2). The combined organic layers were washed with brine (300 mL), dried over Na₂SO₄, filtered, and concentrated under reduced pressure. The crude product was purified by flash chromatography on silica gel (Eluent of 0~10% MeOH in CH₂Cl₂) to give tert-butyl (1-(4-((3-nitropyridin-2-yl)amino) benzyl) piperidin-4-yl) carbamate (30 g, yield: 71%) as a yellow solid. MS: m/z=428.2 [M+H]⁺. ¹H NMR (400 MHz, Chloroform-d) δ 10.10 (s, 1H), 8.51 (dd, J=8.0, 1.6 Hz, 1H), 8.47 (dd, J=8.4, 1.6 Hz, 1H), 7.58 (d, J=8.4 Hz, 2H), 7.32 (d, J=8.4 Hz, 2H), 6.81 (dd, J=8.4, 4.4 Hz, 1H), 4.44 (br s, 1H), 3.47 (s, 2H), 3.44-3.34 (m, 1H), 2.82-2.79 (m, 2H), 2.13-2.05 (m, 2H), 1.93-1.89 (m, 2H), 1.43 (s, 9H), 1.39-1.37 (m, 2H).

### Step 4: tert-Butyl (1-(4-(2-(2-aminopyridin-3-yl)-3H-imidazo[4,5-b]pyridin-3-yl)benzyl)piperidin-4-yl)carbamate

To a solution of (12.5 g, 29.2 mmol) in DMSO (500 mL) was added Na₂S₂O₄ (15.3 g, 87.7 mmol) and 2-aminopyridine-3-carbaldehyde (4.3 g, 35.1 mmol). The mixture was stirred at 100° C. for 16 hr. The reaction mixture was quenched with H₂O (1000 mL) at 20° C. and extracted with EtOAc (1000 mL×2). The combined organic layers were washed with brine (500 mL), dried over Na₂SO₄, filtered, and concentrated under reduced pressure to give a residue. The crude was purified by flash chromatography on silica gel (Eluent of 0~10% MeOH in CH₂Cl₂) to give tert-butyl (1-(4-(2-(2-aminopyridin-3-yl)-3H-imidazo[4,5-b]pyridin-3-yl)benzyl)piperidin-4-yl)carbamate (5.5 g, yield: 38%) as a yellow solid. MS: m/z=500.2 [M+H]⁺. ¹H NMR (400 MHz, Chloroform-d) δ 8.46-8.37 (m, 1H), 8.12-8.02 (m, 2H), 7.48 (d, J=8.0 Hz, 2H), 7.34-7.28 (m, 3H), 7.07 (dd, J=8.0, 4.0 Hz, 1H), 6.62 (br s, 2H), 6.33 (dd, J=7.6, 4.8 Hz, 1H), 4.46 (br d, J=6.0 Hz, 1H), 3.56 (s, 2H), 3.49-3.47 (m, 1H), 2.84 (br d, J=11.2 Hz, 2H), 2.14 (t, J=12.0 Hz, 2H), 1.93 (br d, J=11.2 Hz, 2H), 1.45 (s, 9H), 1.51-1.38 (m, 2H).

### Step 5. 3-(3-(4-((4-Aminopiperidin-1-yl)methyl)phenyl)-3H-imidazo[4,5-b]pyridin-2-yl)pyridin-2-amine

A solution of tert-butyl (1-(4-(2-(2-aminopyridin-3-yl)-3H-imidazo[4,5-b]pyridin-3-yl)benzyl)piperidin-4-yl)carbamate (2.0 g, 4.0 mmol) in HCl/1,4-dioxane (4M, 20 mL) was stirred at 25° C. for 1 hr. The reaction mixture was concentrated under reduced pressure. The residue was quenched with NaHCO₃ (30 mL) at 20° C. and diluted with MeOH. The mixture was filtered, and the filtrate was freeze-dried to give 3-(3-(4-((4-aminopiperidin-1-yl)methyl)phenyl)-3H-imidazo[4,5-b]pyridin-2-yl)pyridin-2-amine (Intermediate 1, 1.45 g, yield: 91%) as a yellow solid. MS: m/z=400.1 [M+H]⁺. ¹H NMR (400 MHz, Dimethylsulfoxide-d₆) δ 8.37-8.28 (m, 1H), 8.19 (dd, J=8.0, 4.0 Hz, 1H), 7.99-7.97 (m, 1H), 7.47-7.32 (m, 5H), 7.15 (dd, J=7.6, 1.6 Hz, 1H), 7.01 (br s, 2H), 6.36 (dd, J=6.8, 4.0 Hz, 1H), 3.50 (s, 2H), 3.27-3.23 (m, 1H), 2.75 (br d, J=10.8 Hz, 2H), 2.00 (t, J=10.8 Hz, 2H), 1.72 (d, J=10.8 Hz, 2H), 1.39-1.31 (m, 2H).

### Intermediate 2: 3-(5-Phenyl-3-(4-(piperazin-1-ylmethyl)phenyl)-3H-imidazo[4,5-b]pyridin-2-yl)pyridin-2-amine

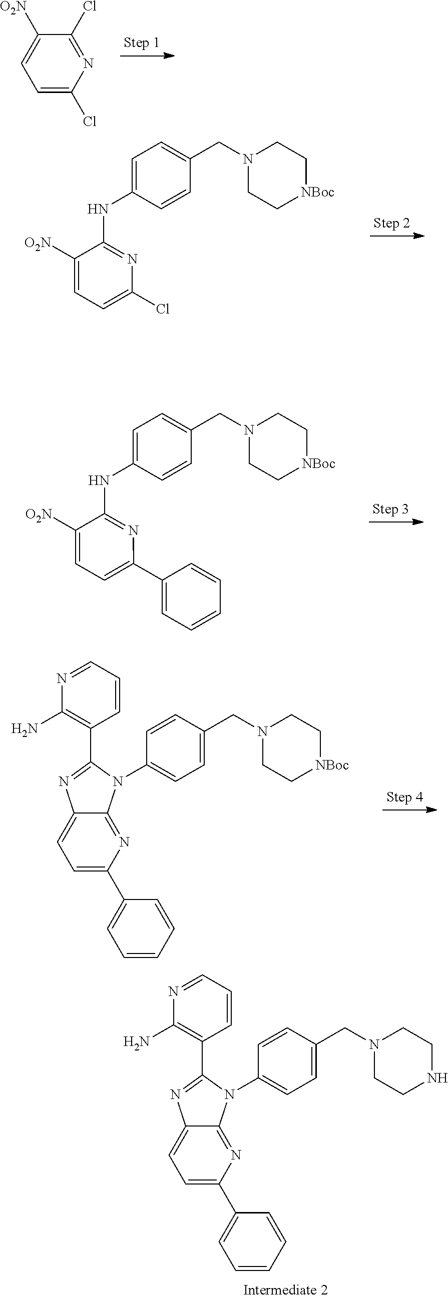

### Step 1: tert-Butyl 4-[[4-[(6-chloro-3-nitro-2-pyridyl)amino]phenyl]methyl]piperazine-1-carboxylate

To a solution of 2,6-dichloro-3-nitro-pyridine (5.0 g, 25.9 mmol) in 1,4-dioxane (50 mL) were added DIEA (6.7 g, 51.8 mmol) and tert-butyl 4-[(4-aminophenyl)methyl]piperazine-1-carboxylate (10.8 g, 25.9 mmol). The mixture was stirred at 60° C. for 12 hr. The reaction mixture was diluted with H₂O (50 mL) and extracted with EtOAc (100 mL×3). The combined organic layers were washed with brine (50 mL×2), dried over Na₂SO₄, filtered, and concentrated under reduced pressure. The residue was purified by silica gel flash chromatography (Eluent of 0~50% EtOAc in petroleum ether) to give tert-butyl 4-[[4-[(6-chloro-3-nitro-2-pyridyl)amino]phenyl]methyl]piperazine-1-carboxylate (6.1 g, yield: 53%) as a yellow solid. MS: m/z=447.9 [M+H]⁺. ¹H NMR (400 MHz, Chloroform-d) δ 10.27 (s, 1H), 8.46 (d, J=8.8 Hz, 1H), 7.61 (d, J=8.4 Hz, 2H), 7.36 (d, J=8.4 Hz, 2H), 6.80 (d, J=8.4 Hz, 1H), 3.52 (s, 2H), 3.48-3.37 (m, 4H), 2.45-2.32 (m, 4H), 1.46 (s, 9H).

### Step 2: tert-Butyl 4-[[4-[(3-nitro-6-phenyl-2-pyridyl)amino]phenyl]methyl]piperazine-1-carboxylate

To a solution of tert-butyl 4-[[4-[(6-chloro-3-nitro-2-pyridyl)amino]phenyl]methyl]piperazine-1-carboxylate (1.0 g, 2.23 mmol) and phenylboronic acid (544 mg, 4.47 mmol) in 1,4-dioxane (10 mL) and H₂O (2 mL) were added Pd(dppf)Cl₂ (327 mg, 0.446 mmol) and K₂CO₃ (926 mg, 6.7 mmol). The mixture was stirred at 60° C. for 4 hr. The reaction mixture was added with H₂O (50 mL) and extracted with EtOAc (100 mL×3). The combined organic layers were washed with brine (50 mL×2), dried over Na₂SO₄, filtered, and concentrated under reduced pressure. The residue was purified by silica gel flash chromatography (Eluent of 0-50% EtOAc in petroleum ether) to give tert-butyl 4-[[4-[(3-nitro-6-phenyl-2-pyridyl)amino]phenyl]methyl]piperazine-1-carboxylate (1.0 g, yield: 92%) as a red solid. MS: m/z=490.1 [M+H]⁺. ¹H NMR (400 MHz, Chloroform-d) δ 10.31 (s, 1H), 8.59 (d, J=8.8 Hz, 1H), 8.06-8.04 (m, 2H), 7.73 (d, J=8.4 Hz, 2H), 7.52-7.44 (m, 3H), 7.37 (d, J=8.4 Hz, 2H), 7.3 (d, J=8.4 Hz, 1H), 3.54 (s, 2H), 3.52-3.37 (m, 4H), 2.49-2.37 (m, 4H), 1.46 (s, 9H).

### Step 3: tert-Butyl 4-[[4-[2-(2-amino-3-pyridyl)-5-phenyl-imidazo[4,5-b]pyridin-3-yl]phenyl]methyl]piperazine-1-carboxylate

To a solution of 2-aminopyridine-3-carbaldehyde (269 mg, 2.21 mmol) and tert-butyl 4-[[4-[(3-nitro-6-phenyl-2-pyridyl)amino]phenyl]methyl]piperazine-1-carboxylate (900 mg, 1.84 mmol) in DMSO (10 mL) was added Na₂S₂O₄ (960 mg, 5.52 mmol) at 15° C. The mixture was stirred at 100° C. for 20 hr. The reaction mixture was diluted with H₂O (50 mL), and the aqueous phase extracted with DCM (80 mL×3). The combined organic layers were washed with brine (80 mL×3), dried over Na₂SO₄, filtered, and concentrated under reduced pressure to give a residue. The residue was purified by silica gel flash chromatography (Eluent of 0~10% MeOH in CH₂Cl₂) to give tert-butyl 4-[[4-[2-(2-amino-3-pyridyl)-5-phenyl-imidazo[4,5-b]pyridin-3-yl]phenyl]methyl]piperazine-1-carboxylate (600 mg, yield: 58%) as a yellow solid. MS: m/z=562.1 [M+H]⁺. ¹H NMR (400 MHz, Chloroform-d) δ 8.13 (d, J=8.4 Hz, 1H), 8.06 (dd, J=5.2, 2.0 Hz, 1H), 8.03-8.01 (m, 2H), 7.81 (d, J=8.4 Hz, 1H), 7.51-7.49 (m, 2H), 7.48-7.35 (m, 5H), 7.10 (dd, J=9.6, 2.0 Hz, 1H), 6.66 (br s, 2H), 6.36 (dd, J=8.0, 4.0 Hz, 1H), 3.64 (s, 2H), 3.53-3.42 (m, 4H), 2.55-2.42 (m, 4H), 1.47 (s, 9H).

### Step 4: 3-(5-Phenyl-3-(4-(piperazin-1-ylmethyl)phenyl)-3H-imidazo[4,5-b]pyridin-2-yl)pyridin-2-amine

A solution of tert-butyl 4-[[4-[2-(2-amino-3-pyridyl)-5-phenyl-imidazo[4,5-b]pyridin-3-yl]phenyl]methyl]piperazine-1-carboxylate (400 mg, 712 μmol) in HCl/1,4-dioxane (4M, 8 mL) was stirred at 25° C. for 4 hr. The reaction was filtered and concentrated under reduced pressure to give 3-[5-phenyl-3-[4-(piperazin-1-ylmethyl)phenyl]imidazo[4,5-b]pyridin-2-yl]pyridin-2-amine (450 mg, HCl) as a yellow solid, which was used in the next step without further purification. The residue (200 mg) was purified by prep-HPLC (column: Waters xbridge 150*25 mm 10 μm; mobile phase: [water(NH₄HCO₃)-ACN]; B %: 19%-49%, 9 min) to give 3-(5-phenyl-3-(4-(piperazin-1-ylmethyl)phenyl)-3H-imidazo[4,5-b]pyridin-2-yl)pyridin-2-amine (Intermediate 2, 49.7 mg) as a yellow solid. MS: m/z=462.2 [M+H]⁺. ¹H NMR (400 MHz, Dimethylsulfoxide-d₆) δ 8.27 (d, J=8.4 Hz, 1H), 8.04-7.97 (m, 4H), 7.48-7.39 (m, 8H), 7.15 (dd, J=8.0, 2.0 Hz, 1H), 7.03 (br s, 2H), 6.38 (dd, J=7.6, 4.8 Hz, 1H), 3.53 (s, 2H), 2.74-2.66 (m, 4H), 2.38-2.27 (m, 4H).

### Intermediate 3: 3-(3-(4-((4-Aminopiperidin-1-yl)methyl)phenyl)-5-phenyl-3H-imidazo[4,5-b]pyridin-2-yl)pyridin-2-amine

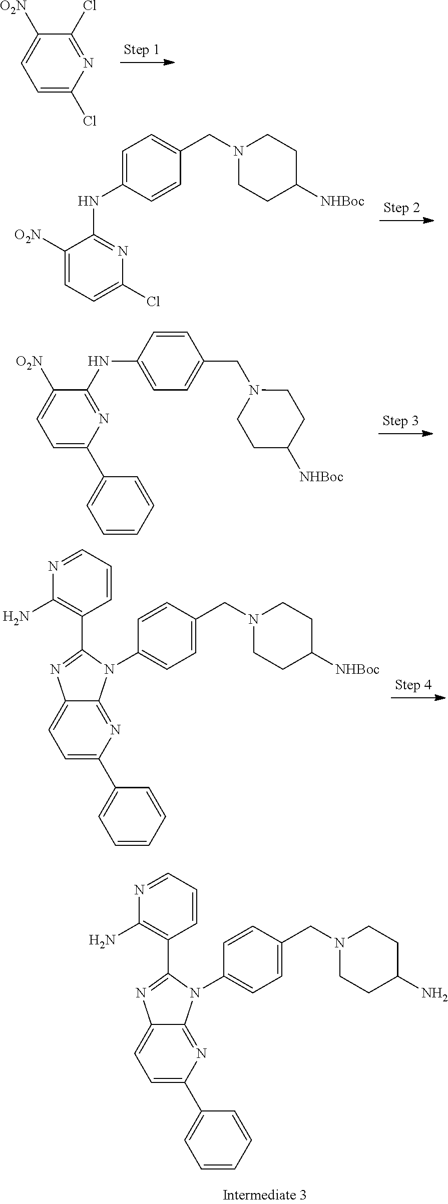

### Step 1: tert-Butyl (1-(4-((6-chloro-3-nitropyridin-2-yl)amino)benzyl)piperidin-4-yl)carbamate

To a solution of 2,6-dichloro-3-nitro-pyridine (3.0 g, 15.5 mmol) and tert-butyl N-[1-[(4-aminophenyl)methyl]-4-piperidyl]carbamate (4.8 g, 15.6 mmol) in 1,4-dioxane (100 mL) was added DIEA (6.0 g, 46.6 mmol). The mixture was stirred at 50° C. for 12 hr. The reaction mixture was diluted with H₂O (50 mL) and extracted with EtOAc (30 mL×2). The combined organic layers were washed with brine (50 mL), dried over Na₂SO₄, filtered, and concentrated under reduced pressure. The residue was purified by silica gel flash chromatography (Eluent of 0~5% MeOH in CH₂Cl₂) to give tert-butyl N-[1-[[4-[(6-chloro-3-nitro-2-pyridyl)amino]phenyl]methyl]-4-piperidyl]carbamate (4.6 g, yield: 64%) as an orange solid. MS: m/z=462.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.42 (br s, 1H), 3.48 (s, 2H), 3.48-3.39 (m, 1H), 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 2: tert-Butyl N-[1-[[4-[(3-nitro-6-phenyl-2-pyridyl)amino]phenyl]methyl]-4-piperidyl]carbamate

A mixture of tert-butyl N-[1-[[4-[(6-chloro-3-nitro-2-pyridyl)amino]phenyl]methyl]-4-piperidyl]carbamate (1.0 g, 2.20 mmol), phenylboronic acid (528 mg, 4.30 mmol), Pd(dppf)Cl₂ (158 mg, 0.216 mmol) and K₂CO₃ (898 mg, 6.50 mmol) in 1,4-dioxane (10 mL) and H₂O (2 mL) was degassed and purged with N₂ three times, and then the mixture was stirred at 100° C. for 12 hr under N₂ atmosphere. After cooling to 25° C., the reaction mixture was diluted with H₂O (50 mL) and extracted with DCM (50 mL×2). The combined organic layers were washed with brine (50 mL), dried over Na₂SO₄, filtered, and concentrated under reduced pressure to give a residue. The residue was purified by silica gel flash chromatography (Eluent of 0~7% MeOH in CH₂Cl₂) to give tert-butyl N-[1-[[4-[(3-nitro-6-phenyl-2-pyridyl)amino]phenyl]methyl]-4-piperidyl]carbamate (1.1 g, yield: 96%) as a yellow solid. MS: m/z=504.1 [M+H]⁺. ¹H NMR (400 MHz, Chloroform-d) δ 10.31 (s, 1H), 8.59 (d, J=8.8 Hz, 1H), 8.09-8.02 (m, 2H), 7.72 (d, J=8.4 Hz, 2H), 7.51-7.47 (m, 3H), 7.36 (d, J=8.8 Hz, 1H), 7.30 (d, J=8.8 Hz, 1H), 4.52-4.37 (m, 1H), 3.52 (s, 2H), 3.51-3.42 (m, 1H), 2.85 (br d, J=11.2 Hz, 2H), 2.12 (br t, J=10.8 Hz, 2H), 1.93 (br d, J=11.0 Hz, 2H), 1.50-1.40 (m, 2H). 1.44 (s, 9H).

### Step 3: tert-Butyl (1-(4-(2-(2-Aminopyridin-3-yl)-5-phenyl-3H-imidazo[4,5-b]pyridin-3-yl)benzyl)piperidin-4-yl) carbamate

To a solution of tert-butyl N-[1-[[4-[(3-nitro-6-phenyl-2-pyridyl)amino]phenyl]methyl]-4-piperidyl]carbamate (200 mg, 0.397 mmol), 2-aminopyridine-3-carbaldehyde (53.4 mg, 0.437 mmol) and Na₂S₂O₄ (207 mg, 1.2 mmol) in DMSO (6 mL). The mixture was stirred at 100° C. for 18 hr. After cooling to 25° C., the reaction mixture was diluted with DCM (40 mL). The organic layers were washed with H₂O (20 mL) and brine (20 mL), dried over Na₂SO₄, filtered and concentrated under reduced pressure to give a residue. The residue was purified by silica gel flash chromatography (IEluent of 0~7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]carbamate (100 mg, yield: 40%) as a yellow solid. MS: m/z=576.2 [M+H]⁺.

### Step 4: 3-(3-(4-((4-Aminopiperidin-1-yl)methyl)phenyl)-5-phenyl-3H-imidazo[4,5-b]pyridin-2-yl)pyridin-2-amine

A solution of tert-butyl (1-(4-(2-(2-aminopyridin-3-yl)-5-phenyl-3H-imidazo[4,5-b]pyridin-3-yl)benzyl)piperidin-4-yl)carbamate (200 mg, 0.347 mmol) in HCl in 1,4-dioxane (4 M, 2 mL) was stirred at 25° C. for 2 hr. The reaction mixture was concentrated under reduced pressure. The residue was purified byprep-HPLC (column: Waters xbridge 150*25 mm 10 μm; mobile phase: [water (NH₄HCO₃)-ACN]; B %: 24%-54%, 8 min) to give 3-(3-(4-((4-aminopiperidin-1-yl)methyl)phenyl)-5-phenyl-3H-imidazo[4,5-b]pyridin-2-yl)pyridin-2-amine (Intermediate 3, 120 mg, yield: 72%) as a light-yellow solid. MS: m/z=476.2 [M+H]⁺. ¹H NMR (400 MHz, Chloroform-d) δ 8.12 (d, J=8.4 Hz, 1H), 8.05 (dd, J=5.2, 1.6 Hz, 1H), 8.01 (d, J=7.2 Hz, 2H), 7.79 (d, J=8.4 Hz, 1H), 7.50-7.35 (m, 7H), 7.09 (dd, J=7.6, 1.2 Hz, 1H), 6.61 (br s, 2H), 6.35 (dd, J=7.6, 4.8 Hz, 1H), 3.58 (s, 2H), 2.88 (br d, J=11.6 Hz, 2H), 2.75-2.65 (m, 1H), 2.09 (br t, J=11.6 Hz, 2H), 1.83 (br d, J=11.6 Hz, 2H), 1.49-1.38 (m, 2H).

### Intermediate 4: 3-(3-(4-(Chloromethyl)phenyl)-5-phenyl-3H-imidazo[4,5-b]pyridin-2-yl)pyridin-2-amine

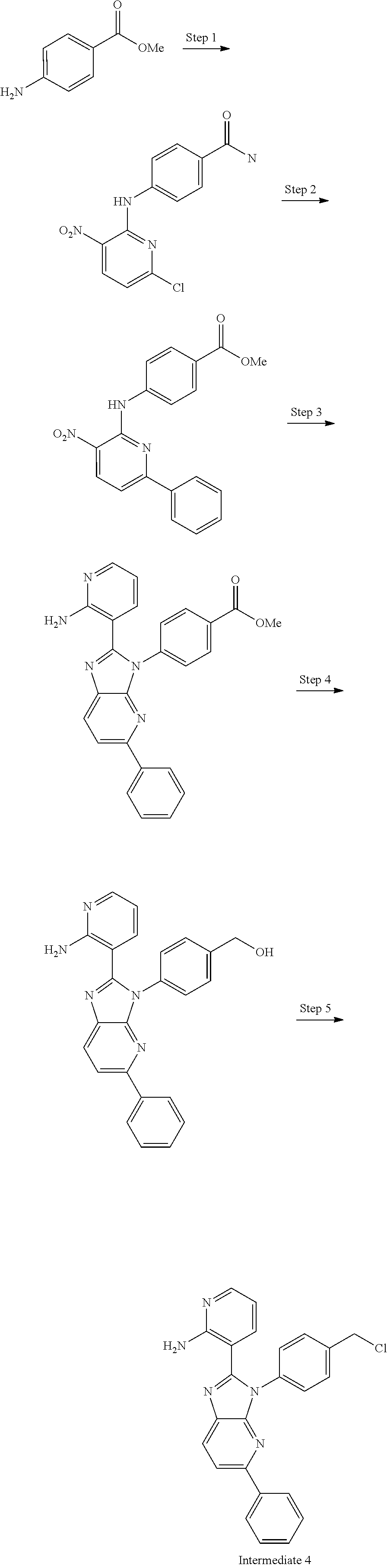 [SMILES: COC(=O)c1ccc(N)cc1 ; NC(=O)c1ccc(Nc2nc(Cl)ccc2[N+](=O)[O-])cc1 ; COC(=O)c1ccc(Nc2nc(-c3ccccc3)ccc2[N+](=O)[O-])cc1 ; COC(=O)c1ccc(-n2c(-c3cccnc3N)nc3ccc(-c4ccccc4)nc32)cc1 ; Nc1ncccc1-c1nc2ccc(-c3ccccc3)nc2n1-c1ccc(CO)cc1 ; Nc1ncccc1-c1nc2ccc(-c3ccccc3)nc2n1-c1ccc(CCl)cc1]

### Step 1: Methyl 4-((6-chloro-3-nitropyridin-2-yl)amino)benzoate

To a solution of methyl 4-aminobenzoate (5 g, 33.1 mmol) in DMSO (50 mL) were added 2,6-dichloro-3-nitro-pyridine (7.66 g, 39.7 mmol) and DIEA (12.82 g, 99.2 mmol). The mixture was stirred at 80° C. for 16 hr. After cooling to 20° C., the reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated. The crude product was triturated with EtOAc at 25° C. for 30 min to give methyl 4-((6-chloro-3-nitropyridin-2-yl)amino)benzoate (8 g, yield: 51%) as a yellow solid. MS: m/z=307.8 [M+H]⁺. ¹H NMR (400 MHz, Dimethylsulfoxide-d₆) δ 10.25 (s, 1H), 8.57 (d, J=8.4 Hz, 1H), 7.97 (d, J=8.8 Hz, 2H), 7.79 (d, J=8.8 Hz, 2H), 7.12 (d, J=8.4 Hz, 1H), 3.85 (s, 3H).

### Step 2: Methyl 4-((3-nitro-6-phenylpyridin-2-yl)amino)benzoate

To a solution of methyl 4-((6-chloro-3-nitropyridin-2-yl)amino)benzoate (45 g, 146 mmol) and phenylboronic acid (21.4 g, 176 mmol) in 1,4-dioxane (500 mL) and H₂O (100 mL) were added Pd(dppf)Cl₂ (10.7 g, 14.6 mmol) and Cs₂CO₃ (143 g, 439 mmol). The mixture was degassed and purged with N₂ three times, and then the mixture was stirred at 80° C. for 16 hr under N₂ atmosphere. The reaction mixture was poured into H₂O (500 mL) and extracted with CH₂Cl₂ (500 mL×3). The combined organic layers were washed with brine (500 mL×3), dried over anhydrous Na₂SO₄, filtered, and concentrated. The crude was triturated with EtOAc at 25° C. for 30 min to give methyl 4-[(3-nitro-6-phenyl-2-pyridyl)amino]benzoate (35.2 g, yield: 69%) as a red solid. MS: m/z=350.0 [M+H]⁺. ¹H NMR (400 MHz, Dimethylsulfoxide-d₆) δ10.25 (s, 1H), 8.62 (d, J=8.8 Hz, 1H), 8.15-8.10 (m, 2H), 8.00 (d, J=8.4 Hz, 2H), 7.92 (d, J=8.4 Hz, 2H), 7.67 (d, J=8.8 Hz, 1H), 7.59-7.54 (m, 3H), 3.86 (s, 3H).

### Step 3: Methyl 4-(2-(2-aminopyridin-3-yl)-5-phenyl-3H-imidazo[4,5-b]pyridin-3-yl)benzoate

To a solution of methyl 4-[(3-nitro-6-phenyl-2-pyridyl)amino]benzoate (15 g, 42.9 mmol) in DMSO (150 mL) were added 2-aminopyridine-3-carbaldehyde (6.29 g, 51.5 mmol) and Na₂S₂O₄ (15 g, 85.9 mmol). The reaction mixture was heated to 100° C. for 16 hr. After cooling to 25° C., the reaction mixture was diluted with H₂O (200 mL) and extracted with CH₂Cl₂ (200 ml×3). The combined organic layers were washed with brine brine (200 ml×2), dried over Na₂SO₄, filtered, and concentrated under reduced pressure. The crude was triturated with CH₂Cl₂ at 25° C. for 30 min to give methyl 4-(2-(2-aminopyridin-3-yl)-5-phenyl-3H-imidazo[4,5-b]pyridin-3-yl)benzoate (12 g, yield: 66%) as a yellow solid. MS: m/z=422.0 [M+H]⁺. ¹H NMR (400 MHz, Dimethylsulfoxide-d₆) δ 8.29 (d, J=8.0 Hz, 1H), 8.12 (d, J=8.4 Hz, 2H), 8.07-8.00 (m, 4H), 7.67 (d, J=8.8 Hz, 2H), 7.49-7.44 (m, 2H), 7.42-7.38 (m, 1H), 7.23 (dd, J=7.6, 1.6 Hz, 1H), 6.89 (br s, 2H), 6.46 (dd, J=7.6, 4.8 Hz, 1H), 3.90 (s, 3H).

### Step 4: (4-(2-(2-Aminopyridin-3-yl)-5-phenyl-3H-imidazo[4,5-b]pyridin-3-yl)phenyl)methanol

To a solution of methyl 4-(2-(2-aminopyridin-3-yl)-5-phenyl-3H-imidazo[4,5-b]pyridin-3-yl)benzoate (2.5 g, 5.9 mmol) in THF (25 mL) was added LiAlH₄ (450 mg, 11.9 mmol) at 0° C. After addition, the resulting mixture was stirred at 25° C. for 2 hr. After the reaction mixture was cooled to 0° C., the reaction mixture was quenched with H₂O (100 mL) and followed by addition of 15% aqueous NaOH (30 mL). Then the reaction mixture was filtered. The filtrate was concentrated to dryness to give (4-(2-(2-Aminopyridin-3-yl)-5-phenyl-3H-imidazo[4,5-b]pyridin-3-yl)phenyl)methanol (1.87 g, yield: 80%) as a yellow solid, which was used in the next step without further purification. MS: m/z=394.1 [M+H]⁺.

### Step 5: 3-(3-(4-(Chloromethyl)phenyl)-5-phenyl-3H-imidazo[4,5-b]pyridin-2-yl)pyridin-2-amine

To a solution of (4-(2-(2-Aminopyridin-3-yl)-5-phenyl-3H-imidazo[4,5-b]pyridin-3-yl)phenyl)methanol (2.3 g, 5.9 mmol) in CH₂Cl₂ (25 mL) was added SOCl₂ (2.1 g, 17.5 mmol). The mixture was stirred at 40° C. for 1 hr. The reaction mixture was filtered. The filtrate was concentrated to dryness to give 3-(3-(4-(chloromethyl)phenyl)-5-phenyl-3H-imidazo[4,5-b]pyridin-2-yl)pyridin-2-amine (Intermediate 4, 1.71 g, yield: 71%) as a yellow solid. MS: m/z=412.0 [M+H]⁺. ¹H NMR (400 MHz, Dimethylsulfoxide-d₆) δ 8.29 (d, J=8.4 Hz, 1H), 8.12 (d, J=8.4 Hz, 2H), 8.07-8.00 (m, 4H), 7.67 (d, J=8.4 Hz, 2H), 7.50-7.44 (m, 2H), 7.42-7.37 (m, 1H), 7.24 (d, J=7.2 Hz, 1H), 6.88 (br s, 2H), 6.46 (dd, J=4.8, 7.6 Hz, 1H), 3.90 (s, 2H).

### Intermediate 5: 3-(3-(4-((2,6-Diazaspiro[3.3]heptan-2-yl)methyl)phenyl)-5-phenyl-3H-imidazo[4,5-b]pyridin-2-yl)pyridin-2-amine

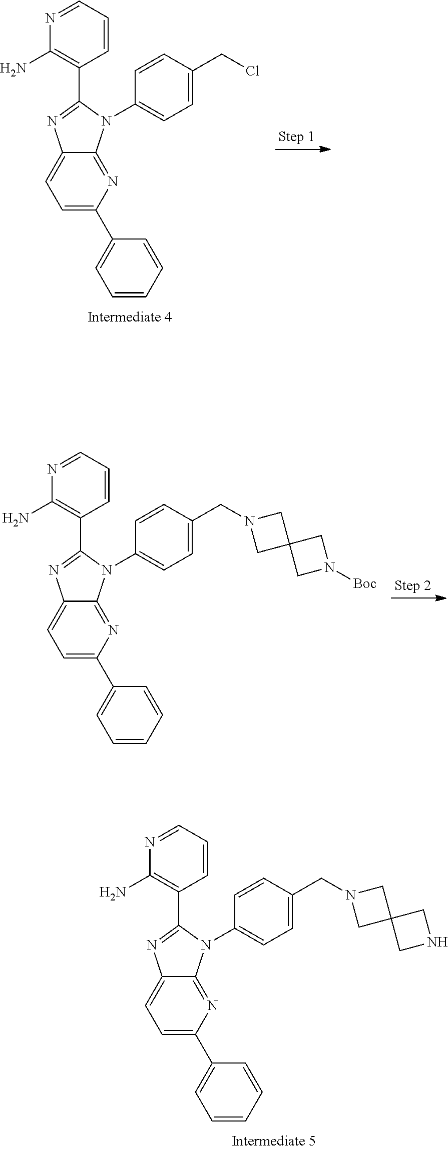 [SMILES: Nc1ncccc1-c1nc2ccc(-c3ccccc3)nc2n1-c1ccc(CCl)cc1 ; CC(C)(C)OC(=O)N1CC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CNC3)C2)cc1]

### Step 1: tert-Butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate

To a solution of Intermediate 4 (200 mg, 486 μmol) in MeCN (3 mL) were added K₂CO₃ (268 mg, 1.94 mmol), tert-butyl 2,6-diazaspiro[3.3]heptane-2-carboxylate (106 mg, 534 μmol) and NaI (7.28 mg, 48 μmol) at 25° C. The reaction mixture was stirred at 80° C. for 3 hr.

The reaction mixture was filtered and concentrated under reduced pressure. The residue was purified by silica gel flash chromatography (Eluent of 0-7% MeOH in CH₂Cl₂) to give tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, yield: 54%) as a yellow solid. MS: m/z=574.6 [M+H]⁺.

### Step 2: 3-(3-(4-((2,6-Diazaspiro[3.3]heptan-2-yl)methyl)phenyl)-5-phenyl-3H-imidazo[4,5-b]pyridin-2-yl)pyridin-2-amine

To a solution of tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, 261 μmol) in CH₂Cl₂ (3 mL) was added TFA (1.54 g, 13.5 mmol) at 25° C. The reaction mixture was stirred at 25° C. for 2 hr. The reaction mixture was concentrated to give 3-(3-(4-((2,6-diazaspiro[3.3]heptan-2-yl)methyl)phenyl)-5-phenyl-3H-imidazo[4,5-b]pyridin-2-yl)pyridin-2-amine (Intermediate 5: 120 mg, TFA salt, yield: 81%) as a yellow solid. MS: m/z=474.2 [M+H]⁺. ¹H NMR (400 MHz, Dimethylsulfoxide-d₆) δ 8.26 (d, J=8.4 Hz, 1H), 8.04-7.97 (m, 4H), 7.49-7.44 (m, 2H), 7.42-7.37 (m, 5H), 7.15 (dd, J=7.6, 1.6 Hz, 1H), 6.99 (br s, 2H), 6.39 (dd, J=8.0, 5.2 Hz, 1H), 3.58 (s, 2H), 3.56 (s, 4H), 3.25 (s, 4H), 1.23 (s, 1H).

### Intermediate 6: (S)-3-(3-(4-((3-Aminopiperidin-1-yl)methyl)phenyl)-5-phenyl-3H-imidazo[4,5-b]pyridin-2-yl)pyridin-2-amine

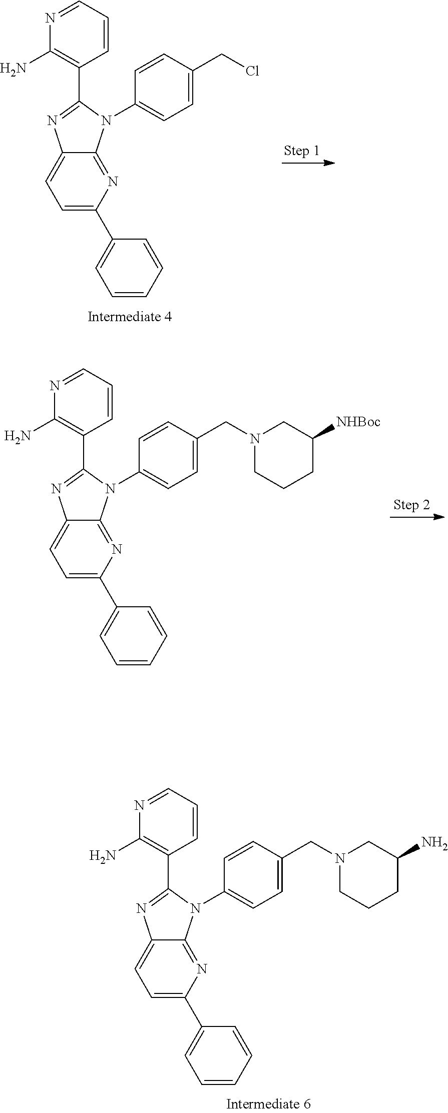 [SMILES: Nc1ncccc1-c1nc2ccc(-c3ccccc3)nc2n1-c1ccc(CCl)cc1 ; CC(C)(C)OC(=O)N[C@H]1CCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCC[C@H](N)C2)cc1]

Intermediate 6 was prepared in a manner similar to Intermediate 5. MS: m/z=476.2 [M+H]⁺. 1H NMR (400 MHz, Methanol-d₄) δ 8.32 (d, J=8.4 Hz, 1H), 8.10-7.98 (m, 4H), 7.94 (d, J=8.4 Hz, 2H), 7.89 (dd, J=7.6, 1.2 Hz, 1H), 7.74 (d, J=8.4 Hz, 2H), 7.49-7.34 (m, 3H), 6.93 (dd, J=7.6, 6.4 Hz, 1H), 4.62 (s, 2H), 3.89-3.56 (m, 3H), 3.28-3.12 (m, 2H), 2.26-1.99 (m, 3H), 1.90-1.66 (m, 1H).

### Intermediate 7: (S)-3-(3-(4-((3-Aminopyrrolidin-1-yl)methyl)phenyl)-5-phenyl-3H-imidazo[4,5-b]pyridin-2-yl)pyridin-2-amine

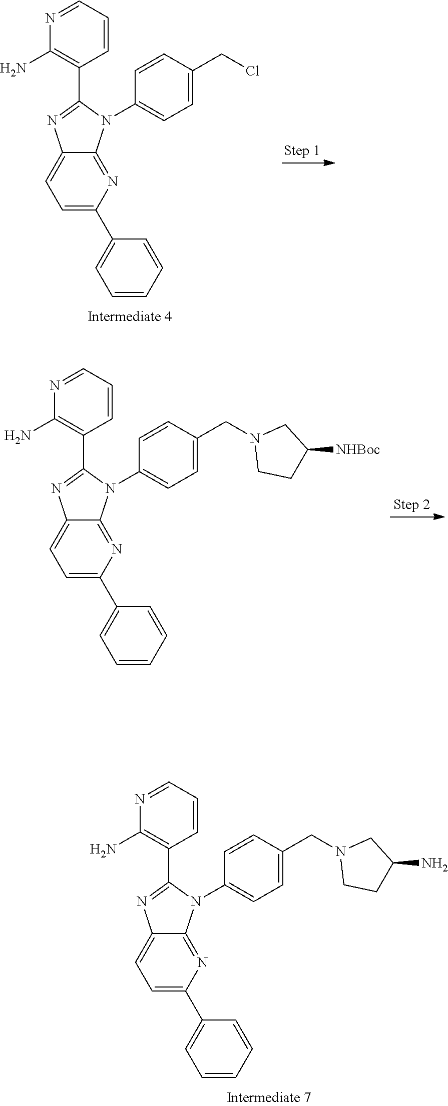 [SMILES: Nc1ncccc1-c1nc2ccc(-c3ccccc3)nc2n1-c1ccc(CCl)cc1 ; CC(C)(C)OC(=O)N[C@H]1CCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CC[C@H](N)C2)cc1]

Intermediate 7 was prepared in a manner similar to Intermediate 5. MS: m/z=462.2 [M+H]⁺. ¹H NMR (400 MHz, Methanol-d₄) δ 8.32 (d, J=8.4 Hz, 1H), 8.07-8.00 (m, 4H), 7.91 (d, J=8.4 Hz, 2H), 7.84 (dd, J=7.6, 1.2 Hz, 1H), 7.72 (d, J=8.4 Hz, 2H), 7.48-7.38 (m, 3H), 6.91 (dd, J=7.2, 7.2 Hz, 1H), 4.68 (s, 2H), 4.32-4.13 (m, 1H), 3.86-3.70 (m, 2H), 3.59-3.33 (m, 2H), 2.93-2.65 (m, 1H), 2.38-2.22 (m, 1H).

### Intermediate 8: 2-(4-(2-(2-Aminopyridin-3-yl)-5-phenyl-3H-imidazo[4,5-b]pyridin-3-yl)benzyl)-2-azaspiro[4.5]decan-8-amine

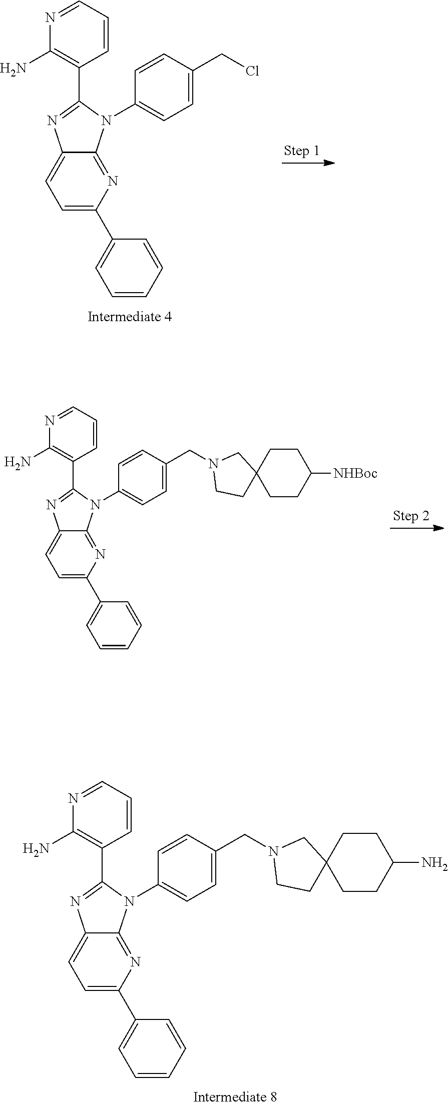 [SMILES: Nc1ncccc1-c1nc2ccc(-c3ccccc3)nc2n1-c1ccc(CCl)cc1 ; CC(C)(C)OC(=O)NC1CCC2(CC1)CCN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CCC3(CCC(N)CC3)C2)cc1]

Intermediate 8 was prepared in a manner similar to Intermediate 5. MS: m/z=530.4 [M+H]⁺. ¹H NMR (400 MHz, Dimethylsulfoxide-d₆) δ 8.26 (d, J=8.8 Hz, 1H), 8.06-7.94 (m, 4H), 7.50-7.37 (m, 7H), 7.15 (d, J=7.2 Hz, 1H), 7.03 (br s, 2H), 6.44-6.32 (m, 1H), 4.40-4.36 (m, 1H), 4.18-4.09 (m, 6H), 1.65-1.45 (m, 6H), 1.31-1.22 (m, 4H).

### Intermediate 9: (R)-3-(3-(4-((3-Aminopiperidin-1-yl)methyl)phenyl)-5-phenyl-3H-imidazo[4,5-b]pyridin-2-yl)pyridin-2-amine

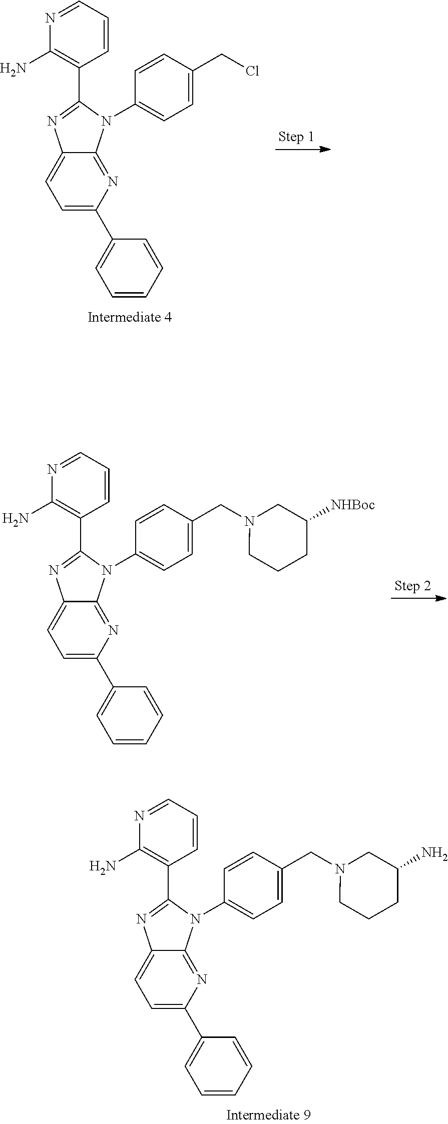 [SMILES: Nc1ncccc1-c1nc2ccc(-c3ccccc3)nc2n1-c1ccc(CCl)cc1 ; CC(C)(C)OC(=O)N[C@@H]1CCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCC[C@@H](N)C2)cc1]

Intermediate 9 was prepared in a manner similar to Intermediate 5. MS: m/z=476.3 [M+H]⁺. ¹H NMR (400 MHz, Dimethylsulfoxide-d₆) δ 8.27 (d, J=8.0 Hz, 1H), 8.04-7.97 (m, 4H), 7.50-7.44 (m, 6H), 7.42-7.38 (m, 1H), 7.16 (dd, J=7.6, 1.6 Hz, 1H), 7.01 (br s, 2H), 6.39 (dd, J=7.6, 4.8 Hz, 1H), 3.62-3.52 (m, 2H), 2.90-2.85 (m, 1H), 2.79-2.74 (m, 1H), 2.62-2.59 (m, 1H), 2.09-2.00 (m, 1H), 1.95-1.87 (m, 1H), 1.80-1.74 (m, 1H), 1.70-1.64 (m, 1H), 1.53-1.45 (m, 1H), 1.26-1.21 (m, 1H).

### Intermediate 10: (R)-3-(3-(4-((3-Aminopyrrolidin-1-yl)methyl)phenyl)-5-phenyl-3H-imidazo[4,5-b]pyridin-2-yl)pyridin-2-amine

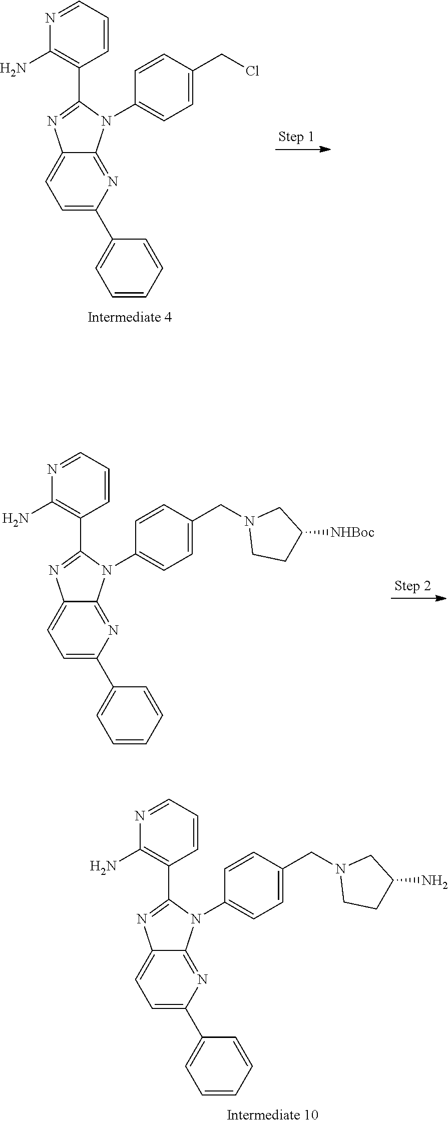 [SMILES: Nc1ncccc1-c1nc2ccc(-c3ccccc3)nc2n1-c1ccc(CCl)cc1 ; CC(C)(C)OC(=O)N[C@@H]1CCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CC[C@@H](N)C2)cc1]

Intermediate 10 was prepared in a manner similar to Intermediate 5. MS: m/z=462.3 [M+H]⁺. ¹H NMR (400 MHz, Dimethylsulfoxide-d₆) δ 8.27 (d, J=8.0 Hz, 1H), 8.04-7.97 (m, 4H), 7.52-7.44 (m, 6H), 7.43-7.34 (m, 1H), 7.6 (dd, J=7.6, 2.0 Hz, 1H), 7.00 (br s, 2H), 6.40 (dd, J=8.0, 5.2 Hz, 1H), 3.69 (s, 2H), 3.60-3.57 (m, 2H), 2.75-2.66 (m, 2H), 2.46-2.40 (m, 1H), 2.18-2.08 (m, 1H), 1.66-1.56 (m, 1H).

### Intermediate 11: 3-(3-(4-((4-Aminopiperidin-1-yl)methyl)phenyl)-6-phenyl-3H-imidazo[4,5-b]pyridin-2-yl)pyridin-2-amine

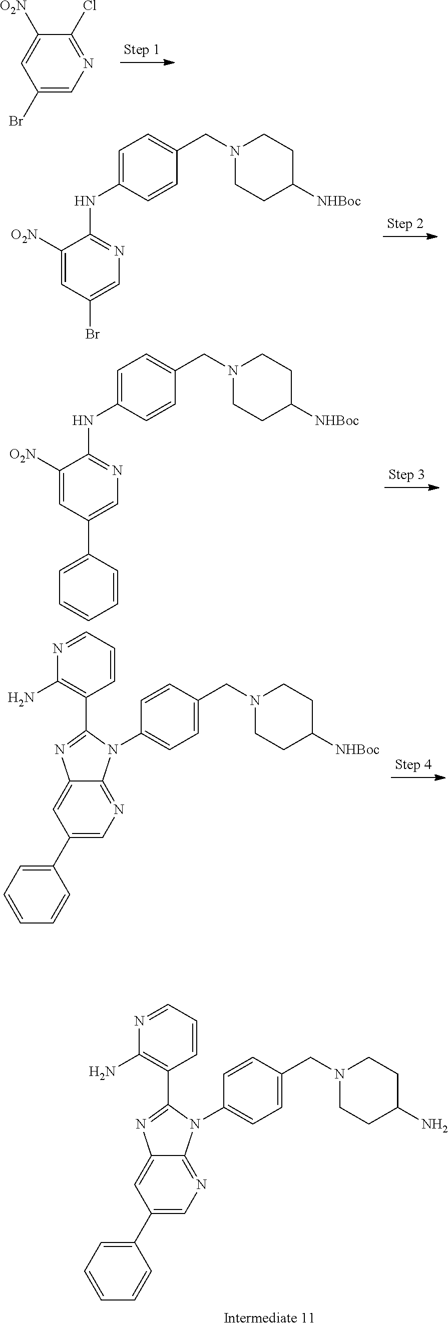

### Step 1: tert-Butyl N-[1-[[4-[(5-bromo-3-nitro-2-pyridyl)amino]phenyl]methyl]-4-piperidyl]carbamate

A mixture of 5-bromo-2-chloro-3-nitro-pyridine (17.1 g, 72 mmol), tert-butyl N-[1-[(4-aminophenyl)methyl]-4-piperidyl]carbamate (22 g, 72 mmol), and DIEA (27.9 g, 216 mmol) in DMSO (200 mL) was stirred at 80° C. for 16 hr. After cooling to 25° C., the mixture was extracted with EtOAc (250 mL×3). The combined organic layers were washed with brine (200 mL×2), dried over anhydrous Na₂SO₄, filtered, and concentrated to give tert-butyl N-[1-[[4-[(5-bromo-3-nitro-2-pyridyl)amino]phenyl]methyl]-4-piperidyl]carbamate (32 g crude) as black brown solid. MS: m/z=506.9, 507.9 [M+H]⁺. ¹H NMR (400 MHz, Chloroform-d) δ 10.04 (s, 1H), 8.64 (d, J=2.0 Hz, 1H), 8.49 (d, J=2.4 Hz, 1H), 7.53 (d, J=8.4 Hz, 2H), 7.33 (d, J=8.4 Hz, 2H), 4.34 (d, J=3.2 Hz, 1H), 3.48 (s, 2H), 3.46-3.35 (m, 1H), 2.82 (br d, J=12.0 Hz, 2H), 2.10 (t, J=10.8 Hz, 2H), 1.91 (br d, J=11.2 Hz, 2H), 1.44 (s, 9H).

### Step 2: tert-Butyl N-[1-[[4-[(3-nitro-5-phenyl-2-pyridyl)amino]phenyl]methyl]-4-piperidyl]carbamate

A mixture of tert-butyl N-[1-[[4-[(5-bromo-3-nitro-2-pyridyl)amino]phenyl]methyl]-4-piperidyl]carbamate (20 g, 39.5 mmol), phenylboronic acid (4.8 g, 39.5 mmol), K₂CO₃ (16.4 g, 118.5 mmol), and Pd(dppf)Cl₂ (1.4 g, 2.0 mmol) in 1,4-dioxane (250 mL) and H₂O (50 mL) was degassed and purged with N₂ three times, and then the mixture was stirred at 80° C. for 16 hr under N₂ atmosphere. After cooling to 25° C., the reaction mixture was filtered and diluted with H₂O (100 mL) and EtOAc (450 mL). The organic phase was separated, washed with brine (100 mL×3), dried over Na₂SO₄, filtered, and concentrated under reduced pressure. The residue was purified by silica gel flash chromatography (Eluent of 3~4% MeOH in CH₂Cl₂) to give tert-butyl-[1-[[4-[(3-nitro-5-phenyl-2-pyridyl)amino]phenyl]methyl]-4-piperidyl]carbamate (8.6 g, yield: 43%) as a red brown soild. MS: m/z=504.2 [M+H]⁺. ¹H NMR (400 MHz, Chloroform-d) δ 10.14 (s, 1H), 8.75 (dd, J=10.0, 2.0 Hz, 2H), 7.62 (d, J=8.0, Hz, 2H), 7.57 (d, J=7.2, Hz, 2H), 7.49 (dd, J=8.0, 8.0 Hz, 2H), 7.43-7.37 (m, 3H), 4.43 (br s, 1H), 3.50 (br s, 2H), 3.49-3.41 (m, 1H), 2.85-2.81 (m, 2H), 2.15-2.08 (m, 2H), 1.94-1.91 (m, 2H), 1.51-1.45 (m, 2H), 1.44 (s, 9H).

### Step 3: tert-Butyl N-[1-[[4-[2-(2-amino-3-pyridyl)-6-phenyl-imidazo[4,5-b]pyridin-3-yl]phenyl]methyl]-4-piperidyl]carbamate

A mixture of tert-butyl N-[1-[[4-[(3-nitro-5-phenyl-2-pyridyl)amino]phenyl]methyl]-4-piperidyl]carbamate (2.2 g, 4.4 mmol), 2-aminopyridine-3-carbaldehyde (694 mg, 5.7 mmol), and Na₂S₂O₄ (1.5 g, 8.7 mmol) in DMSO (100 mL) was degassed and purged with N₂ three times, and then the mixture was stirred at 100° C. for 16 hr under N₂ atmosphere. After cooling to 25° C., the reaction mixture was filtered and diluted with H₂O (30 mL) and EtOAc (45 mL). The organic phase was separated, washed with brine (10 mL×3), dried over Na₂SO₄, filtered and concentrated under reduced pressure. The residue was purified by silica gel flash chromatography (Eluent of 5~6% EtOAc in petroleum ether) to give tert-butyl N-[1-[[4-[2-(2-amino-3-pyridyl)-6-phenyl-imidazo[4,5-b]pyridin-3-yl]phenyl]methyl]-4-piperidyl]carbamate (700 mg, yield: 28%) as a black brown soild. MS: m/z=576.2 [M+H]⁺. ¹H NMR (400 MHz, Chloroform-d) δ 8.63 (d, J=2.0 Hz, 1H), 8.27 (d, J=2.0 Hz, 1H), 8.07 (dd, J=5.2, 2.0 Hz, 1H), 7.67-7.62 (m, 2H), 7.54-7.47 (m, 4H), 7.44-7.39 (m, 1H), 7.36 (d, J=8.4 Hz, 2H), 7.09 (dd, J=8.0, 2.0 Hz, 1H), 6.65 (br s, 2H), 6.35 (dd, J=8.0, 4.8 Hz, 1H), 4.45 (br s, 1H), 3.58 (s, 2H), 3.55-3.46 (m, 1H), 2.92-2.77 (m, 2H), 2.16 (br t, J=10 Hz, 2H), 1.95 (br d, J=11.2 Hz, 2H), 1.63-1.56 (m, 2H), 1.45 (s, 9H).

### Step 4: 3-(3-(4-((4-Aminopiperidin-1-yl)methyl)phenyl)-6-phenyl-3H-imidazo[4,5-b]pyridin-2-yl)pyridin-2-amine

A mixture of tert-butyl N-[1-[[4-[2-(2-amino-3-pyridyl)-6-phenyl-imidazo[4,5-b]pyridin-3-yl]phenyl]methyl]-4-piperidyl]carbamate (2.4 g, 4.17 mmol) in HCl/1,4-dioxane (4M, 20 mL) and MeOH (4 mL) was stirred at 25° C. for 2 hr. The reaction mixture was filtered to give residue (2 g HCl salt, yield: 94.3%). The residue (100 mg) was purified by prep-HPLC (column: Welch Ultimate C18 150*25 mm 5 μm; mobile phase: [water (FA)-ACN]; B %: 0% to 25%, 10 min) to give 3-(3-(4-((4-aminopiperidin-1-yl)methyl)phenyl)-6-phenyl-3H-imidazo[4,5-b]pyridin-2-yl)pyridin-2-amine (Intermediate 11, 26.6 mg, 2HCOOH salt, yield: 94%) as a yellow solid. MS: m/z=476.1 [M+H]⁺. ¹H NMR (400 MHz, Dimethylsulfoxide-d₆) δ 8.63 (d, J=2.0 Hz, 1H), 8.47 (d, J=2.0 Hz, 1H), 8.34 (s, 2H), 8.00 (dd, J=4.8, 1.6 Hz, 1H), 7.78 (d, J=7.6 Hz, 2H), 7.53 (dd, J=7.6, 7.6 Hz, 2H), 7.47-7.39 (m, 5H), 7.20 (dd, J=7.6, 1.6 Hz, 1H), 7.04 (br s, 2H), 6.39 (dd, J=7.6, 4.8 Hz, 1H), 3.55 (s, 2H), 2.98-2.88 (m, 1H), 2.87-2.80 (m, 2H), 2.09-1.97 (m, 2H), 1.90-1.80 (m, 2H), 1.57-1.44 (m, 2H).

### Intermediate 12: 3-(3-(4-((1,4-Diazepan-1-yl)methyl)phenyl)-5-phenyl-3H-imidazo[4,5-b]pyridin-2-yl)pyridin-2-amine

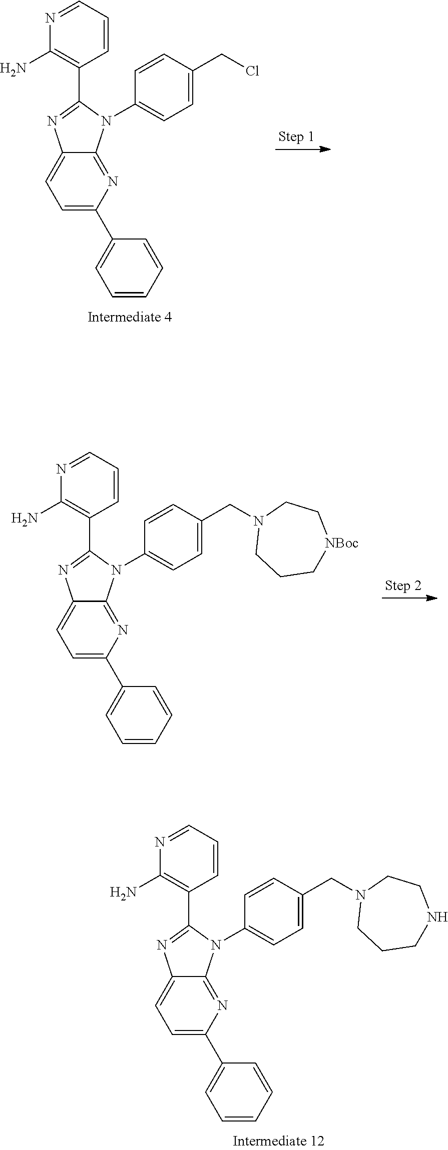 [SMILES: Nc1ncccc1-c1nc2ccc(-c3ccccc3)nc2n1-c1ccc(CCl)cc1 ; CC(C)(C)OC(=O)N1CCCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCCNCC2)cc1]

Intermediate 12 was prepared in a manner similar to Intermediate 5. MS: m/z=476.3 [M+H]⁺. ¹H NMR (400 MHz, Dimethylsulfoxide-d₆) δ 8.4 (d, J=8.4 Hz, 1H), 8.04-7.97 (m, 4H), 7.52-7.39 (m, 7H), 7.14 (dd, J=7.6, 1.8 Hz, 1H), 7.05 (br s, 2H), 6.39-6.34 (m, 1H), 3.72 (s, 2H), 2.82 (t, J=6.4 Hz, 2H), 2.78-2.74 (m, 2H), 2.67 (t, J=6.4 Hz, 2H), 2.66-2.57 (m, 2H), 1.78-1.64 (m, 2H).

### Intermediate 13: 7-(4-(2-(2-Aminopyridin-3-yl)-5-phenyl-3H-imidazo[4,5-b]pyridin-3-yl)benzyl)-7-azaspiro[3.5]nonan-2-amine

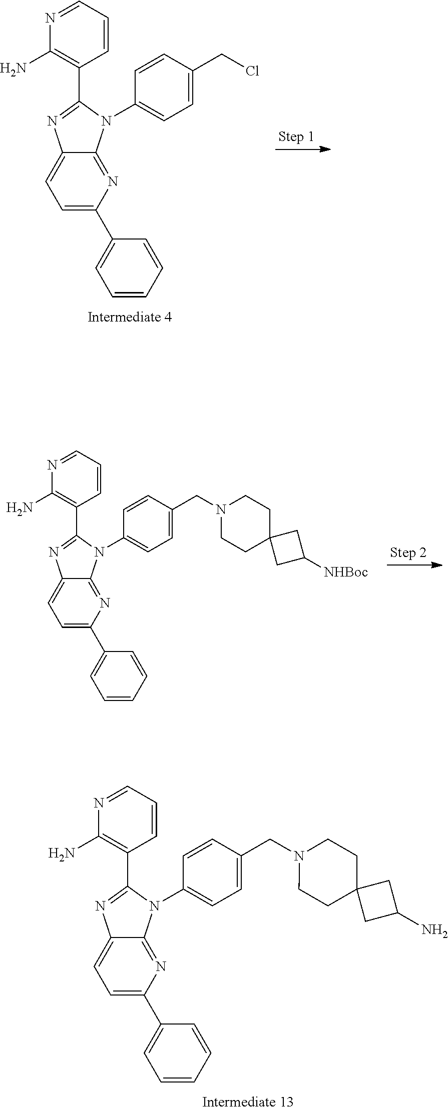 [SMILES: Nc1ncccc1-c1nc2ccc(-c3ccccc3)nc2n1-c1ccc(CCl)cc1 ; CC(C)(C)OC(=O)NC1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CC(N)C3)cc1]

Intermediate 13 was prepared in a manner similar to Intermediate 5. MS: m/z=516.3 [M+H]⁺. ¹H NMR (400 MHz, Dimethylsulfoxide-d₆) δ 8.27 (d, J=8.0 Hz, 1H), 8.06-7.95 (m, 4H), 7.55-7.35 (m, 7H), 7.18-7.12 (m, 1H), 7.01 (br s, 2H), 6.36 (dd, J=7.6, 4.8 Hz, 1H), 3.71-3.58 (m, 1H), 3.52 (s, 2H), 3.01-2.98 (m, 4H), 2.41-2.20 (m, 2H), 2.14-2.05 (m, 2H), 1.90-1.80 (m, 2H), 1.67-1.53 (m, 2H).

Intermediate 14: N-(3-(3-(4-((4-aminopiperidin-1-yl)methyl)phenyl)-2-(2-aminopyridin-3-yl)-3H-imidazo[4,5-b]pyridin-5-yl)phenyl)acetamide

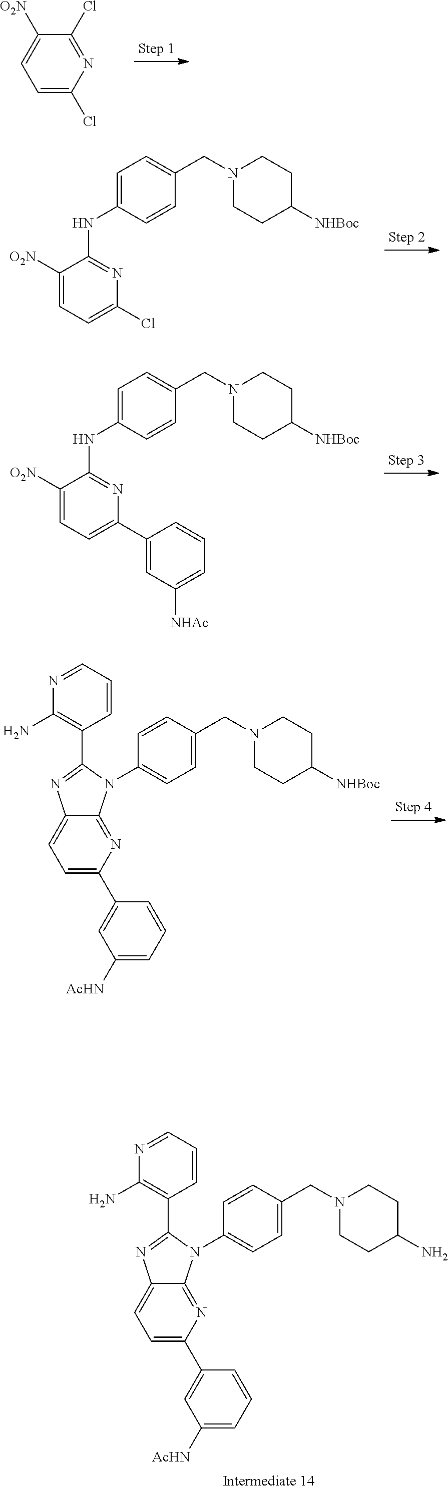 [SMILES: O=[N+]([O-])c1ccc(Cl)nc1Cl ; CC(C)(C)OC(=O)NC1CCN(Cc2ccc(Nc3nc(Cl)ccc3[N+](=O)[O-])cc2)CC1 ; CC(=O)Nc1cccc(-c2ccc([N+](=O)[O-])c(Nc3ccc(CN4CCC(NC(=O)OC(C)(C)C)CC4)cc3)n2)c1 ; CC(=O)Nc1cccc(-c2ccc3nc(-c4cccnc4N)n(-c4ccc(CN5CCC(NC(=O)OC(C)(C)C)CC5)cc4)c3n2)c1 ; CC(=O)Nc1cccc(-c2ccc3nc(-c4cccnc4N)n(-c4ccc(CN5CCC(N)CC5)cc4)c3n2)c1]

### Step 1: tert-Butyl (1-(4-((6-chloro-3-nitropyridin-2-yl)amino)benzyl)piperidin-4-yl)carbamate

To a solution of 2,6-dichloro-3-nitro-pyridine (3.0 g, 15.5 mmol) and tert-butyl N-[1-[(4-aminophenyl)methyl]-4-piperidyl]carbamate (4.8 g, 15.6 mmol) in 1,4-dioxane (100 mL) was added DIEA (6.0 g, 46.6 mmol). The mixture was stirred at 50° C. for 12 hr. The reaction mixture was diluted with H₂O (50 mL) and extracted with EtOAc (30 mL×2). The combined organic layers were washed with brine (50 mL), dried over Na₂SO₄, filtered, and concentrated under reduced pressure. The residue was purified by silica gel flash chromatography (Eluent of 0~5% MeOH in CH₂Cl₂) to give tert-butyl N-[1-[[4-[(6-chloro-3-nitro-2-pyridyl)amino]phenyl]methyl]-4-piperidyl]carbamate (4.6 g, yield: 64%) as an orange solid. MS: m/z=462.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.42 (br s, 1H), 3.48 (s, 2H), 3.48-3.39 (m, 1H), 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 2: tert-Butyl (1-(4-((6-(3-acetamidophenyl)-3-nitropyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.16 mmol), (3-acetamidophenyl)boronic acid (773 mg, 4.32 mmol), Pd(dppf)Cl₂ (158 mg, 0.216 mmol) and K₂CO₃ (895 mg, 6.48 mmol) in H₂O (2 mL) and 1,4-dioxane (10 mL) was degassed and purged with N₂ three times, and then the mixture was stirred at 100° C. for 12 hr under N₂ atmosphere. The reaction mixture was diluted with H₂O (50 mL) and extracted with CH₂Cl₂ (50 mL×2). The combined organic layers were washed with brine (50 mL), dried over Na₂SO₄, filtered, and concentrated under reduced pressure to give a residue. The residue was purified by silica gel flash chromatography (Eluent of 0~5% MeOH in CH₂Cl₂) to give tert-butyl N-[1-[[4-[[6-(3-acetamidophenyl)-3-nitro-2-pyridyl]amino]phenyl]methyl]-4-piperidyl]carbamate (1.04 g, yield: 86%) as an orange solid. MS: m/z=561.1 [M+H]⁺. ¹H NMR (400 MHz, Chloroform-d) δ 10.30 (s, 1H), 8.57 (d, J=8.8 Hz, 1H), 8.26 (br s, 1H), 7.79 (d, J=8.0 Hz, 1H), 7.72 (d, J=8.4 Hz, 2H), 7.63-7.57 (m, 1H), 7.47-7.35 (m, 4H), 7.28 (d, J=8.8 Hz, 1H), 4.44 (br s, 1H), 3.51 (s, 2H), 3.44-3.48 (m, 1H), 2.93-2.78 (m, 2H), 2.23 (s, 3H), 2.13 (br t, J=10.4 Hz, 2H), 1.93 (br d, J=11.2 Hz, 2H), 1.52-1.45 (m, 2H), 1.44 (s, 9H).

### Step 3: tert-Butyl N-[1-[[4-[5-(3-acetamidophenyl)-2-(2-amino-3-pyridyl)imidazo[4,5-b]pyridin-3-yl]phenyl]methyl]-4-piperidyl]carbamate

A solution of tert-butyl N-[1-[[4-[[6-(3-acetamidophenyl)-3-nitro-2-pyridyl]amino]phenyl]methyl]-4-piperidyl]carbamate (950 mg, 1.69 mmol), 2-aminopyridine-3-carbaldehyde (228 mg, 1.9 mmol) and Na₂SO₄ (590 mg, 3.4 mmol) in DMSO (12 mL) was stirred at 100° C. for 18 hr. After cooling to 25° C., the reaction mixture was diluted with CH₂Cl₂ (50 mL). The organic layers were washed with H₂O (50 mL) and brine (50 mL), dried over Na₂SO₄, filtered, and concentrated under reduced pressure. The residue was purified by silica gel flash chromatography (Eluent of 0~7% MeOH in CH₂Cl₂) to give tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (536 mg, yield: 50%) as a yellow solid. MS: m/z=633.3 [M+H]⁺. ¹H NMR (400 MHz, Chloroform-d) δ 8.11 (d, J=8.4 Hz, 1H), 8.06 (dd, J=4.8, 2.0 Hz, 1H), 8.00 (br s, 1H), 7.78 (d, J=8.4 Hz, 1H), 7.73 (d, J=8.0 Hz, 1H), 7.66 (d, J=8.0 Hz, 1H), 7.49 (br s, 1H), 7.45 (d, J=8.4 Hz, 2H), 7.40-7.34 (m, 3H), 7.05 (dd, J=8.0, 1.6 Hz, 1H), 6.60 (br s, 2H), 6.33 (dd, J=7.6, 4.8 Hz, 1H), 4.47 (br s, 1H), 3.58 (s, 2H), 3.51-3.48 (m, 1H), 2.85 (br d, J=11.2 Hz, 2H), 2.18-2.16 (m, 2H), 2.14 (s, 3H), 1.94 (br d, J=10.8 Hz, 2H), 1.49-1.47 (m, 2H), 1.45 (s, 9H).

### Step 4: N-(3-(3-(4-((4-Aminopiperidin-1-yl)methyl)phenyl)-2-(2-aminopyridin-3-yl)-3H-imidazo[4,5-b]pyridin-5-yl)phenyl)acetamide

A solution of tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (300 mg, 474 μmol) in HCl/1,4-dioxane (4 M, 2 mL) was stirred at 25° C. for 2 hr. The pH of the reaction mixture was adjusted to abount 8 by addition of NaHCO₃. The mixture was extracted with DCM (15 mL×3). The combined organic layers were washed with brine (10 mL×2), dried over Na₂SO₄, filtered and concentrated under reduced pressure to give N-(3-(3-(4-((4-aminopiperidin-1-yl)methyl)phenyl)-2-(2-aminopyridin-3-yl)-3H-imidazo[4,5-b]pyridin-5-yl)phenyl)acetamide (Intermediate 14, 134 mg, yield: 53%) as a yellow solid. MS: m/z=533.2 [M+H]⁺. ¹H NMR (400 MHz, Chloroform-d) δ 8.13 (d, J=8.4 Hz, 1H), 8.07 (dd, J=4.8, 1.6 Hz, 1H), 8.02 (s, 1H), 7.81 (d, J=8.4 Hz, 1H), 7.76 (d, J=8.0 Hz, 1H), 7.69 (d, J=8.0 Hz, 1H), 7.48 (d, J=8.4 Hz, 2H), 7.42-7.37 (m, 4H), 7.08 (d, J=6.8 Hz, 1H), 6.61 (br s, 2H), 6.36 (dd, J=7.6, 4.8 Hz, 1H), 3.59 (s, 2H), 2.90 (br d, J=11.2 Hz, 2H), 2.77-2.67 (m, 1H), 2.20 (s, 3H), 2.12 (br t, J=11.2 Hz, 2H), 1.85 (br d, J=11.2 Hz, 2H), 1.49-1.41 (m, 2H).

### Intermediate 15: 3-(3-(4-((4-(Methylamino)piperidin-1-yl)methyl)phenyl)-5-phenyl-3H-imidazo[4,5-b]pyridin-2-yl)pyridin-2-amine

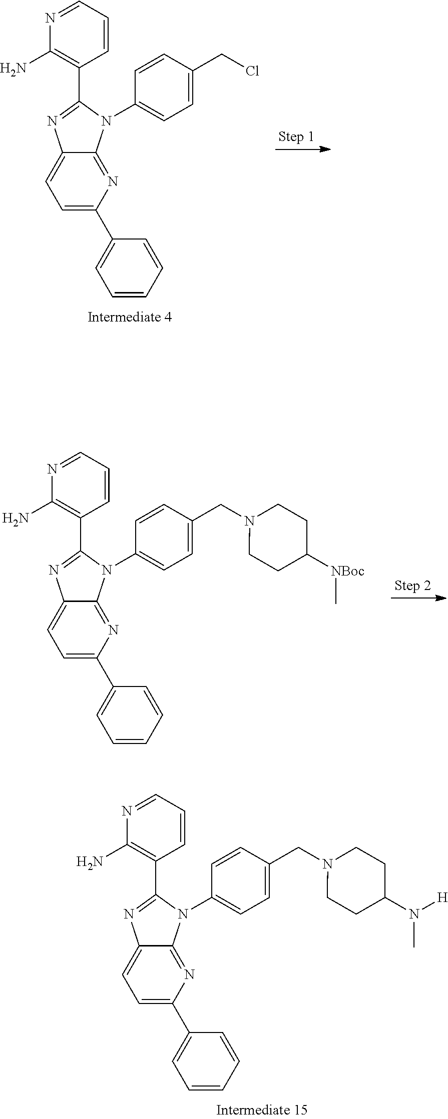 [SMILES: Nc1ncccc1-c1nc2ccc(-c3ccccc3)nc2n1-c1ccc(CCl)cc1 ; CN(C(=O)OC(C)(C)C)C1CCN(Cc2ccc(-n3c(-c4cccnc4N)nc4ccc(-c5ccccc5)nc43)cc2)CC1 ; CNC1CCN(Cc2ccc(-n3c(-c4cccnc4N)nc4ccc(-c5ccccc5)nc43)cc2)CC1]

Intermediate 15 was prepared in a manner similar to Intermediate 5. MS: m/z=490.2 [M+H]⁺. ¹H NMR (400 MHz, Dimethylsulfoxide-d₆) δ 8.27 (d, J=8.4 Hz, 1H), 8.07-7.94 (m, 4H), 7.53-7.43 (m, 6H), 7.41-7.39 (m, 1H), 7.16 (dd, J=7.8, 1.2 Hz, 1H), 7.02 (br s, 2H), 6.37 (dd, J=7.4, 4.8 Hz, 1H), 3.55 (s, 2H), 3.47-3.37 (m, 1H), 2.80 (br d, J=12.0 Hz, 2H), 2.31 (s, 3H), 2.01 (dd, J=11.4, 9.8 Hz, 2H), 1.85-1.77 (m, 2H), 1.36-1.22 (m, 2H).

### Intermediate 16: 3-(3-(4-((2,7-Diazaspiro[3.5]nonan-7-yl)methyl)phenyl)-5-phenyl-3H-imidazo[4,5-b]pyridin-2-yl)pyridin-2-amine

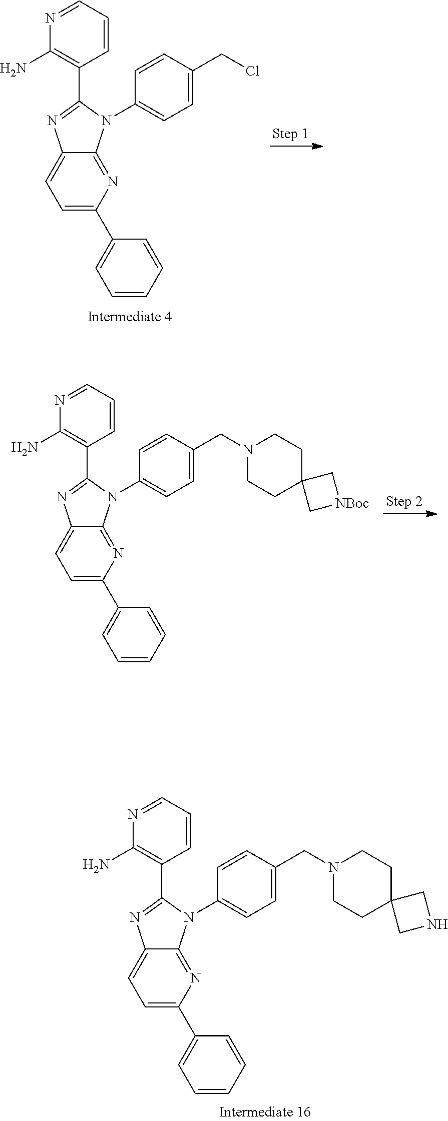 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CNC3)cc1]

Intermediate 16 was prepared in a manner similar to Intermediate 5. MS: m/z=502.3 [M+H]⁺. ¹H NMR (400 MHz, Dimethylsulfoxide-d₆) δ 8.26 (d, J=8.4 Hz, 1H), 8.05-7.97 (m, 4H), 7.49-7.39 (m, 7H), 7.18-7.12 (dd, J=7.6, 1.6 Hz, 1H), 7.02 (br s, 2H), 6.36 (dd, J=7.6, 5.2 Hz, 1H), 3.58-3.41 (m, 6H), 2.38-2.26 (m, 4H), 1.71-1.68 (m, 4H).

### Intermediate 17: 3-(3-(4-((2,8-Diazaspiro[4.5]decan-8-yl)methyl)phenyl)-5-phenyl-3H-imidazo[4,5-b]pyridin-2-yl)pyridin-2-amine

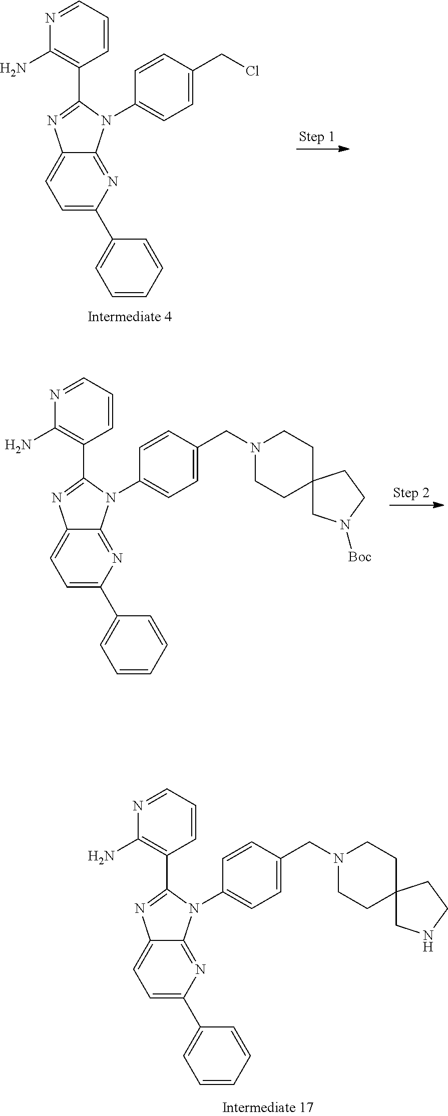 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCNC3)CC2)cc1]

Intermediate 17 was prepared in a manner similar to Intermediate 5. MS: m/z=516.3 [M+H]⁺. ¹H NMR (400 MHz, Methanol-d₆) δ 8.18 (d, J=8.4 Hz, 1H), 8.02 (d, J=7.2 Hz, 2H), 7.97 (dd, J=4.8, 1.6 Hz, 1H), 7.93 (d, J=8.4 Hz, 1H), 7.52 (d, J=8.4 Hz, 2H), 7.45-7.34 (m, 5H), 7.31 (dd, J=6.8, 1.6 Hz, 1H), 6.45 (dd, J=7.6, 4.8 Hz, 1H), 3.62 (s, 2H), 3.10 (t, J=7.2 Hz, 2H), 2.85 (s, 2H), 2.50 (m, 4H), 1.75 (t, J=7.2 Hz, 2H), 1.65 (t, J=5.6 Hz, 4H).

### Intermediate 18: 3-(3-(4-((3,9-diazaspiro[5.5]undecan-3-yl)methyl)phenyl)-5-phenyl-3H-imidazo[4,5-b]pyridin-2-yl)pyridin-2-amine

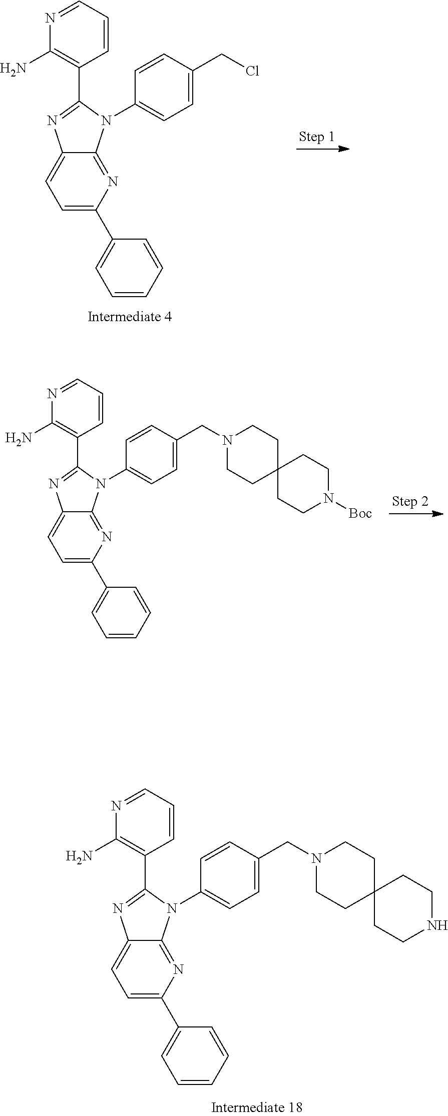 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)CC1 ; Nc1ncccc1-c1nc2ccc(-c3ccccc3)nc2n1-c1ccc(CN2CCC3(CCNCC3)CC2)cc1]

### Step 1: tert-Butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate

To a solution of Intermediate 4 (600 mg, 1.46 mmol) and tert-butyl 3,9-diazaspiro[5.5]undecane-3-carboxylate (408 mg, 1.60 mmol) in DMF (5 mL) were added NaI (21.9 mg, 146 μmol) and K₂CO₃ (403 mg, 2.91 mmol). The mixture was stirred at 80° C. for 16 hr. The reaction mixture was poured into H₂O (15 mL) and extracted with EtOA (20 mL×3). The combined organic layers were dried over anhydrous Na₂SO₄, filtered, and concentrated under reduced pressure. The residue was purified by silica gel flash chromatography (Eluent of 0~65% EtOAc in petroleum ether) to give tert-butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate (450 mg, yield: 49%) as a yellow solid. MS: m/z=630.5 [M+H]⁺.

### Step 2: 3-(3-(4-((3,9-Diazaspiro[5.5]undecan-3-yl)methyl)phenyl)-5-phenyl-3H-imidazo[4,5-b]pyridin-2-yl)pyridin-2-amine

To a solution of 9-(4-(2-(2-aminopyridin-3-yl)-5-phenyl-3H-imidazo[4,5-b]pyridin-3-yl)benzyl)-3,9-diazaspiro[5.5]undecane-3-carboxylate (200 mg, 318 μmol) in 1,4-dioxane (2 mL) was added 4M HCl in 1,4-dioxane (2 mL). The mixture was stirred at 25° C. for 1 hr. The reaction mixture was filtered, and the filter cake was dried to give 3-(3-(4-((3,9-diazaspiro[5.5]undecan-3-yl)methyl)phenyl)-5-phenyl-3H-imidazo[4,5-b]pyridin-2-yl)pyridin-2-amine (Intermediate 18, 121 mg, HCl salt, yield: 67%) as a yellow solid. MS: m/z=530.3 [M+H]⁺. ¹H NMR (400 MHz, Dimethylsulfoxide-d₆) δ 10.81-10.50 (m, 1H), 8.79-8.66 (m, 2H), 8.38 (dd, J=8.4 Hz, 1H), 8.13-8.11 (m, 1H), 8.08-8.05 (m, 4H), 7.86-7.77 (m, 3H), 7.67 (d, J=8.0 Hz, 2H), 7.51-7.46 (m, 2H), 7.45-7.41 (m, 1H), 6.86-6.78 (m, 1H), 4.41 (d, J=4.4 Hz, 2H), 3.21-3.15 (m, 2H), 3.09-3.01 (m, 6H), 1.91-1.85 (m, 2H), 1.82-1.71 (m, 4H), 1.54-1.53 (m, 2H).

### Intermediate 19: 3-(3-(4-((2,6-diazaspiro[3.4]octan-6-yl)methyl)phenyl)-5-phenyl-3H-imidazo[4,5-b]pyridin-2-yl)pyridin-2-amine

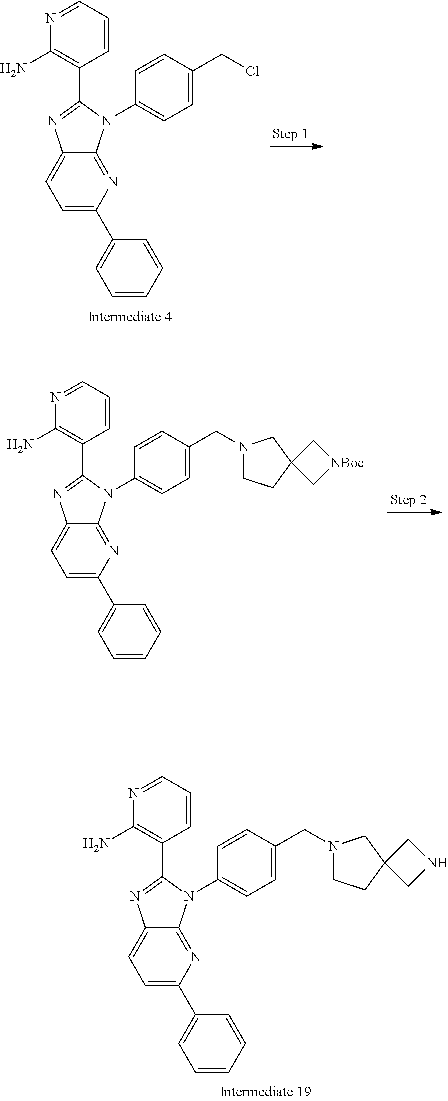 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CNC3)C2)cc1]

Intermediate 19 was prepared in a manner similar to Intermediate 5. MS: m/z=488.3 [M+H]⁺. ¹H NMR (400 MHz, Methanol-d₄) δ 8.18 (d, J=8.4 Hz, 1H), 8.06-7.89 (m, 4H), 7.51 (d, J=8.0 Hz, 2H), 7.45-7.40 (m, 3H), 7.39-7.30 (m, 3H), 6.48 (dd, J=7.6, 5.2 Hz, 1H), 3.99 (d, J=2.4 Hz, 2H), 3.72 (s, 2H), 2.87 (s, 2H), 2.71-2.61 (m, 2H), 2.18 (t, J=7.2 Hz, 2H), 1.28-1.25 (m, 2H).

### Intermediate 20: 3-(3-(4-((2,7-Diazaspiro[4.4]nonan-2-yl)methyl)phenyl)-5-phenyl-3H-imidazo[4,5-b]pyridin-2-yl)pyridin-2-amine

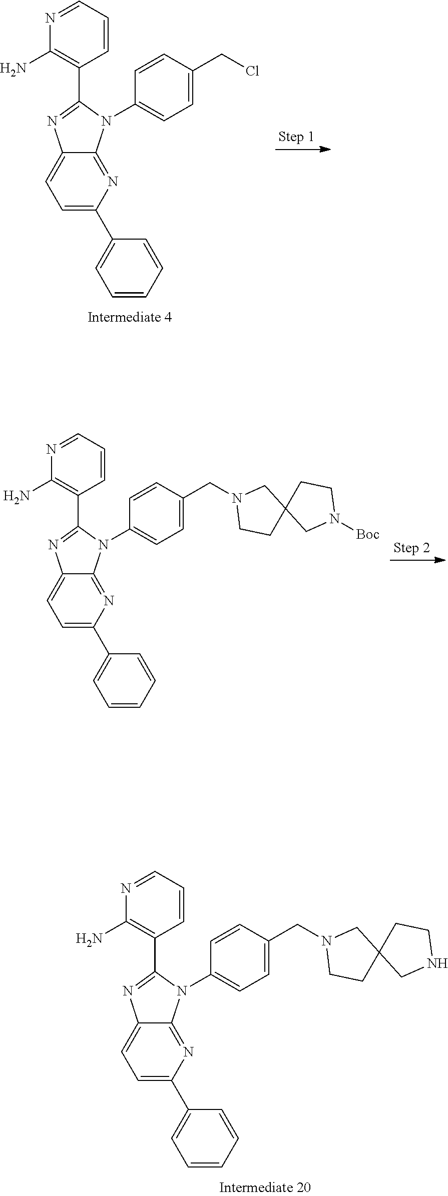 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCNC3)C2)cc1]

Intermediate 20 was prepared in a manner similar to Intermediate 18. MS: m/z=502.3 [M+H]⁺. ¹H NMR (400 MHz, Dimethylsulfoxide-d₆) δ 11.93 (br s, 1H), 9.88-9.48 (m, 2H), 8.60-8.35 (m, 2H), 8.38 (d, J=8.4 Hz, 1H), 8.16 (dd, J=6.4, 1.6 Hz, 1H), 8.08-8.05 (m, 3H), 7.98-7.83 (m, 3H), 7.66 (d, J=8.4 Hz, 2H), 7.52-7.40 (m, 3H), 7.02-6.92 (m, 1H), 4.54-4.43 (m, 2H), 3.64-3.47 (m, 2H), 3.43-3.27 (m, 3H), 3.25-3.18 (m, 3H), 2.31-2.14 (m, 2H), 2.11-1.96 (m, 2H).

### Intermediate 21: 3-(3-(4-((2,8-diazaspiro[4.5]decan-2-yl)methyl)phenyl)-5-phenyl-3H-imidazo[4,5-b]pyridin-2-yl)pyridin-2-amine

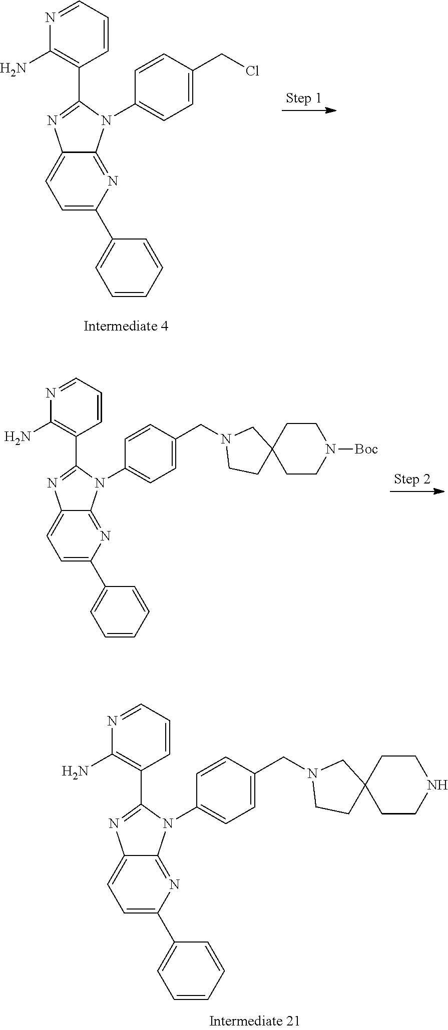 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)CC1 ; Nc1ncccc1-c1nc2ccc(-c3ccccc3)nc2n1-c1ccc(CN2CCC3(CCNCC3)C2)cc1]

Intermediate 21 was prepared in a manner similar to Intermediate 18. MS: m/z=516.3 [M+H]⁺. ¹H NMR (400 MHz, Methanol-d₄) δ 8.33 (d, J=8.8 Hz, 1H), 8.10-8.01 (m, 4H), 7.96-7.87 (m, 3H), 7.72 (d, J=8.0 Hz, 2H), 7.49-7.40 (m, 3H), 6.96-6.87 (m, 1H), 4.67-4.54 (m, 2H), 3.75-3.65 (m, 2H), 3.51 (d, J=10.0 Hz, 1H), 3.35 (s, 2H), 3.32-3.20 (m, 3H), 2.32-2.23 (m, 1H), 2.15-2.05 (m, 2H), 2.04-1.96 (m, 3H).

### Intermediate 22: 3-(3-(4-((2,7-diazaspiro[4.5]decan-2-yl)methyl)phenyl)-5-phenyl-3H-imidazo[4,5-b]pyridin-2-yl)pyridin-2-amine

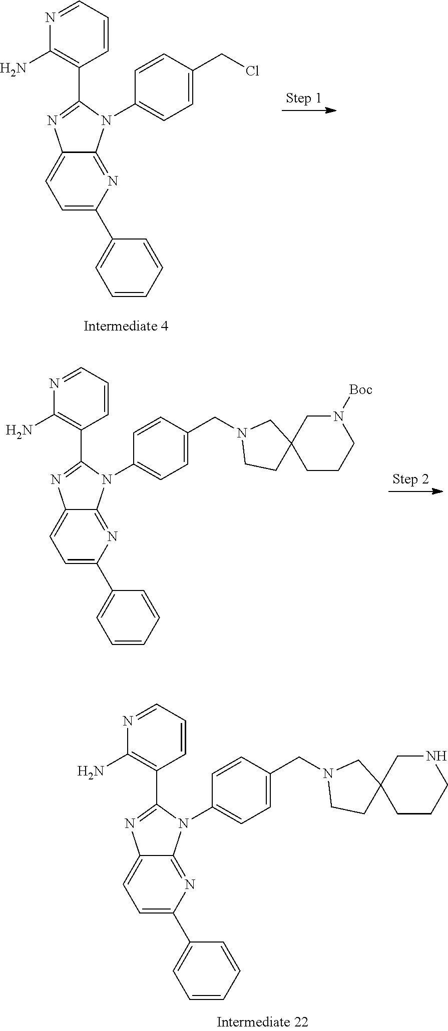 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCCNC3)C2)cc1]

Intermediate 22 was prepared in a manner similar to Intermediate 18. MS: m/z=516.4 [M+H]⁺. ¹H NMR (400 MHz, Methanol-d₄) δ 8.32 (d, J=8.4 Hz, 1H), 8.09-8.00 (m, 4H), 7.93 (d, J=8.4 Hz, 2H), 7.89 (d, J=7.2 Hz, 1H), 7.72 (d, J=8.0 Hz, 2H), 7.49-7.37 (m, 3H), 6.99-6.85 (m, 1H), 4.62 (d, J=11.6 Hz, 2H), 3.78-3.72 (m, 1H), 3.65-3.47 (m, 2H), 3.44-3.38 (m, 1H), 3.35 (s, 2H), 3.18-3.13 (m, 2H), 2.26-2.13 (m, 1H), 2.11-2.01 (m, 1H), 1.95-1.86 (m, 4H).

### Intermediate 23: 3-(3-(4-((2,7-Diazaspiro[3.5]nonan-2-yl)methyl)phenyl)-5-phenyl-3H-imidazo[4,5-b]pyridin-2-yl)pyridin-2-amine

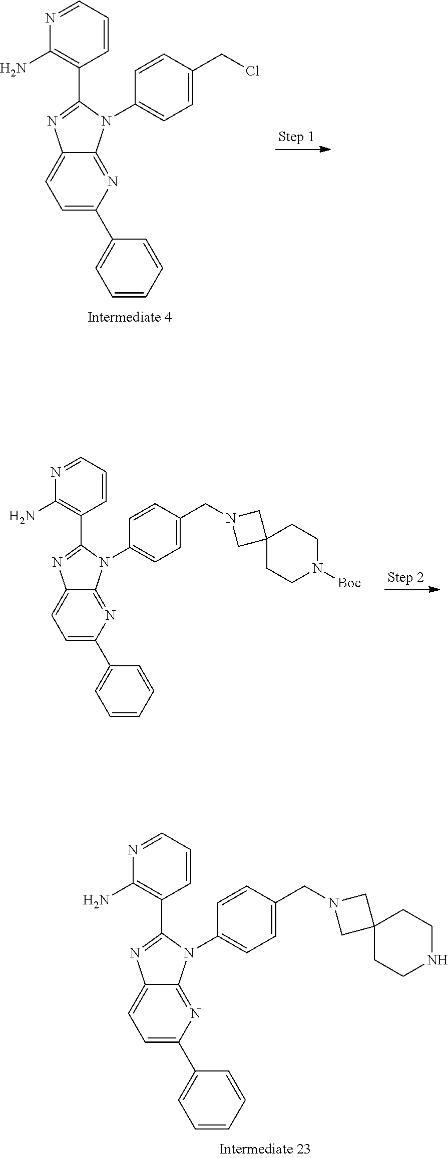 [SMILES: Nc1ncccc1-c1nc2ccc(-c3ccccc3)nc2n1-c1ccc(CCl)cc1 ; CC(C)(C)OC(=O)N1CCC2(CC1)CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3(CCNCC3)C2)cc1]

Intermediate 23 was prepared in a manner similar to Intermediate 18. MS: m/z=502.3 [M+H]⁺. ¹H NMR (400 MHz, Dimethylsulfoxide-d₆) δ 11.79-11.63 (m, 1H), 9.00-8.82 (m, 2H), 8.36 (d, J=8.8 Hz, 1H), 8.33-8.18 (m, 1H), 8.13 (dd, J=6.8, 1.2 Hz, 1H), 8.04-8.08 (m, 3H), 7.83-7.75 (m, 3H), 7.65 (d, J=8.4 Hz, 2H), 7.52-7.46 (m, 2H), 7.45-7.39 (m, 1H), 6.92-6.83 (m, 1H), 4.50 (d, J=6.0 Hz, 2H), 3.96 (d, J=6.0 Hz, 4H), 3.07-3.10 (m, 2H), 2.95-3.02 (m, 2H), 2.10-2.17 (m, 2H), 1.97-2.04 (m, 2H).

### Intermediate 24: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

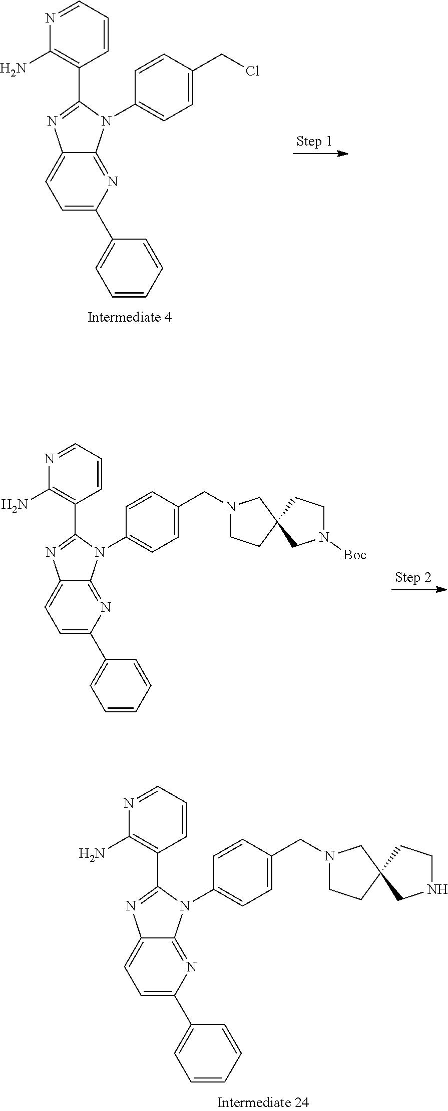 [SMILES: Nc1ncccc1-c1nc2ccc(-c3ccccc3)nc2n1-c1ccc(CCl)cc1 ; CC(C)(C)OC(=O)N1CC[C@@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@@]3(CCNC3)C2)cc1]

Intermediate 24 was prepared in a manner similar to Intermediate 18. MS: m/z=502.3 [M+H]⁺. ¹H NMR (400 MHz, Dimethylsulfoxide-d₆) δ 12.00-11.77 (m, 1H), 9.73-9.52 (m, 2H), 8.58-8.44 (m, 1H), 8.38 (d, J=8.4 Hz, 1H), 8.16 (dd, J=6.0, 1.6 Hz, 1H), 8.04-8.09 (m, 3H), 7.92-7.85 (m, 3H), 7.66 (d, J=8.4 Hz, 2H), 7.52-7.46 (m, 2H), 7.45-7.40 (m, 1H), 7.00-6.92 (m, 1H), 4.57-4.41 (m, 2H), 3.50-3.58 (m, 4H), 3.41-3.33 (m, 2H), 3.25-3.22 (m, 2H), 2.28-2.13 (m, 2H), 2.10-1.97 (m, 2H).

### Intermediate 25: 3-(3-(4-(2,9-Diazaspiro[5.5]undecan-9-ylmethyl)phenyl)-5-phenyl-3H-imidazo[4,5-b]pyridin-2-yl)pyridin-2-amine

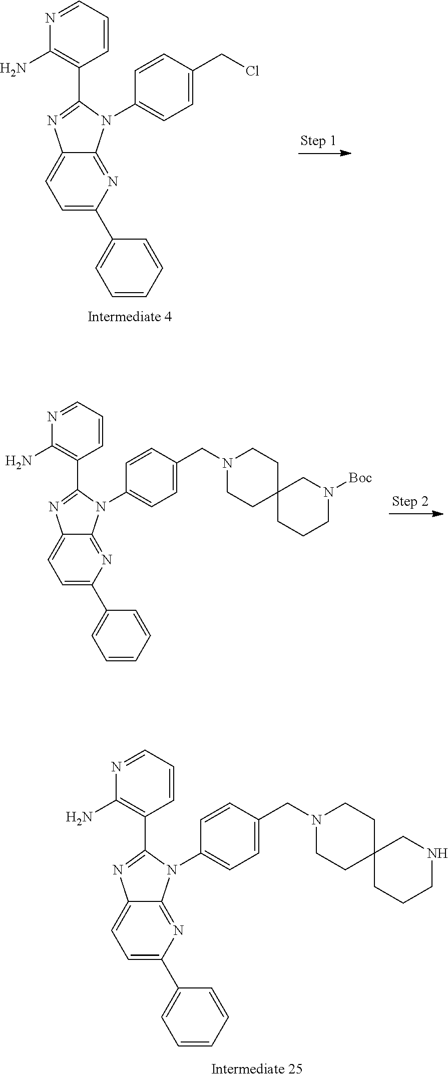 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCCNC3)CC2)cc1]

### Step 1: tert-Butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylate

To a solution of Intermediate 4 in DMF (10 mL) were added K₂CO₃ (671 mg, 4.86 mmol) and tert-butyl 2,9-diazaspiro[5.5]undecane-2-carboxylate (679 mg, 2.67 mmol). The mixture was stirred at 25° C. for 48 hr. The reaction mixture was diluted with H₂O (10 ml) at 25° C. and extracted with CH₂Cl₂ (20 mL×3). The combined organic layers were washed with brine (15 mL), dried over Na₂SO₄, filtered, and concentrated under reduced pressure. The crude was purified by silica gel flash chromatography (Eluent of 1~4% MeOH in CH₂Cl₂) to give tert-butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylat (Intermediate 26, 612 mg, yield: 40%) as a yellow solid. MS: m/z=630.5 [M+H]⁺. ¹H NMR (400 MHz, Dimethylsulfoxide-d₆) δ 8.26 (d, J=8.4 Hz, 1H), 8.06-7.94 (m, 4H), 7.48-7.33 (m, 7H), 7.14 (dd, J=7.6, 1.6 Hz, 1H), 7.05 (br s, 2H), 6.35 (dd, J=7.2, 5.2 Hz, 1H), 3.62-3.55 (m, 2H), 3.27 (s, 2H), 3.22-3.15 (m, 2H), 2.49-2.44 (m, 2H), 2.35-2.22 (m, 2H), 1.38-1.34 (m, 8H), 1.38 (s, 1H).

### Step 2: 3-(3-(4-(2,9-Diazaspiro[5.5]undecan-9-ylmethyl)phenyl)-5-phenyl-3H-imidazo[4,5-b]pyridin-2-yl)pyridin-2-amine

A solution of tert-butyl 9-[[4-[2-(2-amino-3-pyridyl)-5-phenyl-imidazo[4,5-b]pyridin-3-yl]phenyl]methyl]-2,9-diazaspiro[5.5]undecane-2-carboxylate (630 mg, 1.0 mmol) in HCl/1,4-dioxane (4M, 6 mL) was stirred at 25° C. for 1 hr. The reaction was concentrated under reduced pressure to give 3-(3-(4-(2,9-diazaspiro[5.5]undecan-9-ylmethyl)phenyl)-5-phenyl-3H-imidazo[4,5-b]pyridin-2-yl)pyridin-2-amine (Intermediate 25, 540 mg, HCl salt, 9.0 mg delivered) as a yellow solid. MS: m/z=530.4 [M+H]⁺. ¹H NMR (400 MHz, Dimethylsulfoxide-d₆) δ 11.24-10.95 (m, 1H), 9.45-8.99 (m, 2H), 8.52 (br s, 1H), 8.38 (d, J=8.4 Hz, 1H), 8.16 (d, J=6.0 Hz, 1H), 8.12-8.02 (m, 3H), 7.91-7.81 (m, 3H), 7.73-7.62 (m, 2H), 7.52-7.40 (m, 3H), 6.93-6.84 (m, 1H), 4.41 (d, J=5.2 Hz, 2H), 3.21-3.09 (m, 5H), 2.99-2.90 (m, 2H), 2.87-2.75 (m, 1H), 2.13-2.09 (m, 1H), 2.00-1.88 (m, 1H), 1.86-1.73 (m, 2H), 1.72-1.56 (m, 3H), 1.45-1.40 (m, 1H).

### Intermediate 26: 3-(3-(4-(((3S,5S)-3,5-Dimethylpiperazin-1-yl)methyl)phenyl)-5-phenyl-3H-imidazo[4,5-b]pyridin-2-yl)pyridin-2-amine

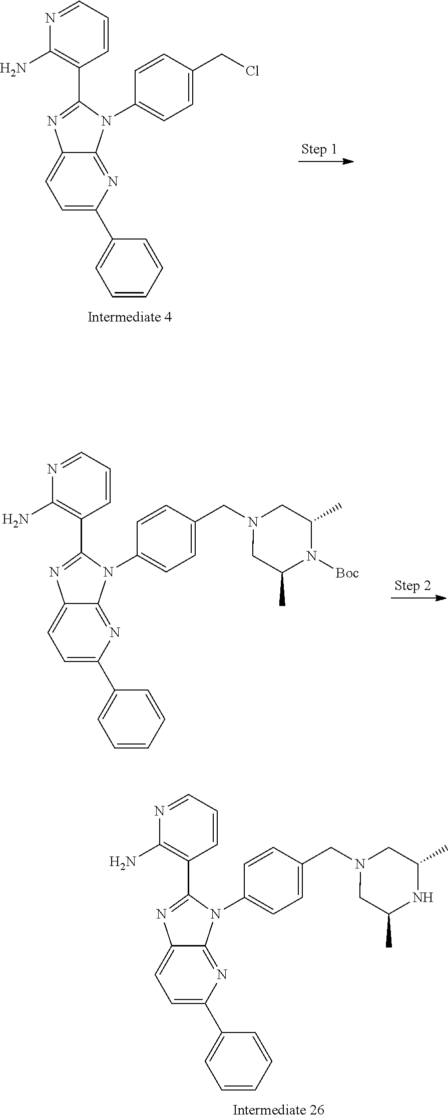 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)C[C@H](C)N1C(=O)OC(C)(C)C ; C[C@H]1CN(Cc2ccc(-n3c(-c4cccnc4N)nc4ccc(-c5ccccc5)nc43)cc2)C[C@H](C)N1]

Intermediate 26 was prepared in a manner similar to Intermediate 25. MS: m/z=490.6 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (dd, J=7.6, 1.6 Hz, 1H), 7.04 (br s, 2H), 6.40-6.34 (m, 1H), 3.54 (d, J=14.0 Hz, 1H), 3.45-3.40 (m, 1H), 3.10-3.04 (m, 2H), 2.41-2.33 (m, 2H), 2.06-2.00 (m, 2H), 1.04 (d, J=6.4 Hz, 6H).

### Intermediate 27: 3-(3-(4-(((3R,5R)-3,5-Dimethylpiperazin-1-yl)methyl)phenyl)-5-phenyl-3H-imidazo[4,5-b]pyridin-2-yl)pyridin-2-amine

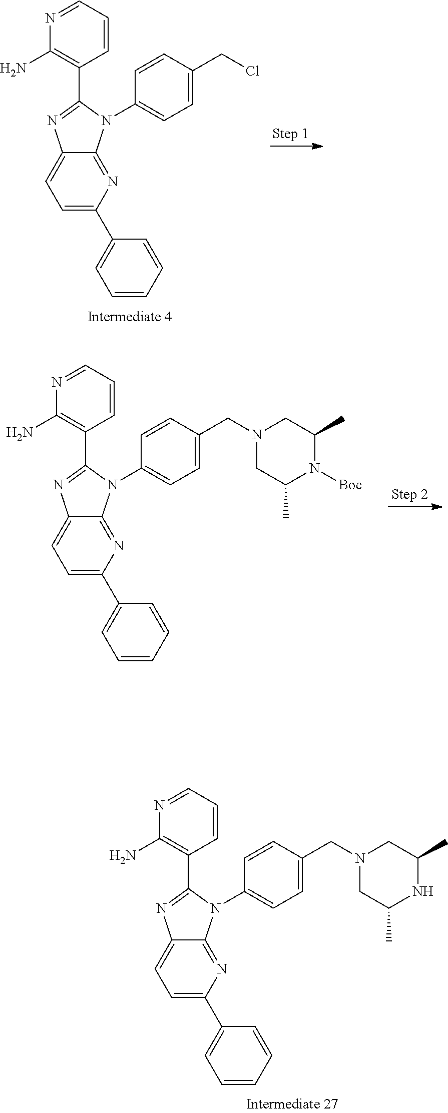 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)C[C@@H](C)N1C(=O)OC(C)(C)C ; C[C@@H]1CN(Cc2ccc(-n3c(-c4cccnc4N)nc4ccc(-c5ccccc5)nc43)cc2)C[C@@H](C)N1]

### Step 1: tert-Butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 4 (1.06 g, 2.57 mmol) and tert-butyl (2R,6R)-2,6-dimethylpiperazine-1-carboxylate (500 mg, 2.33 mmol) in DMF (10 mL) was added DIEA (905 mg, 7.0 mmol). The mixture was stirred at 80° C. for 16 hr. The mixture was quenched with H₂O (40 mL) and extracted with EtOAc (40 mL×3). The combined organic phases were dried over anhydrous Na₂SO₄, filtered, and concentrated. The residue was purified by silica gel flash chromatography (Eluent of 0~9% MeOH in CH₂Cl₂) to give tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (Intermediate 27, 1 g, yield: 69.4%) as a yellow solid. MS: m/z=590.4 [M+H]⁺. ¹H NMR (400 MHz, Dimethylsulfoxide-d₆) δ 8.27 (d, J=8.4 Hz, 1H), 8.05-7.98 (m, 4H), 7.52-7.43 (m, 6H), 7.42-7.36 (m, 1H), 7.16 (dd, J=7.6, 1.6 Hz, 1H), 7.04 (br s, 2H), 6.37 (dd, J=7.6, 4.8 Hz, 1H), 3.83-3.74 (m, 2H), 3.65 (d, J=13.6 Hz, 1H), 3.48 (d, J=13.6 Hz, 1H), 3.35-3.39 (m, 2H), 2.27-2.19 (m, 2H), 1.42-1.41 (m, 1H), 1.40 (s, 9H), 1.23 (d, J=6.4 Hz, 6H).

### Step 2: 3-(3-(4-(((3R,5R)-3,5-Dimethylpiperazin-1-yl)methyl)phenyl)-5-phenyl-3H-imidazo[4,5-b]pyridin-2-yl)pyridin-2-amine

A solution of tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (100 mg, 169 μmol) in HCl/1,4-dioxane (4M, 1 mL) was stirred at 25° C. for 0.5 hr. The mixture was filtered to give 3-(3-(4-(((3R,5R)-3,5-dimethylpiperazin-1-yl)methyl)phenyl)-5-phenyl-3H-imidazo[4,5-b]pyridin-2-yl)pyridin-2-amine (Intermediate 27, 84 mg HCl salt, yield: 95%) as a yellow solid. MS: m/z=490.2 [M+H]⁺. ¹H NMR (400 MHz, Dimethylsulfoxide-d₆) δ 12.41-11.57 (m, 0.5H), 10.79-10.27 (m, 0.5H), 10.07-9.71 (m, 1H), 8.65-8.51 (m, 1H), 8.38 (d, J=8.4 Hz, 1H), 8.16 (d, J=6.0 Hz, 1H), 8.11-7.95 (m, 4H), 7.93-7.82 (m, 3H), 7.68 (d, J=8.0 Hz, 2H), 7.51-7.42 (m, 3H), 7.04 (t, J=6.8 Hz, 1H), 4.50-4.25 (m, 2H), 3.44-3.32 (m, 2H), 3.20-3.01 (m, 2H), 2.91-2.87 (m, 1H), 2.75-2.71 (m, 1H), 1.60-1.31 (m, 6H).

### Intermediate 28: (R)-3-(3-(4-((3-Methylpiperazin-1-yl)methyl)phenyl)-5-phenyl-3H-imidazo[4,5-b]pyridin-2-yl)pyridin-2-amine

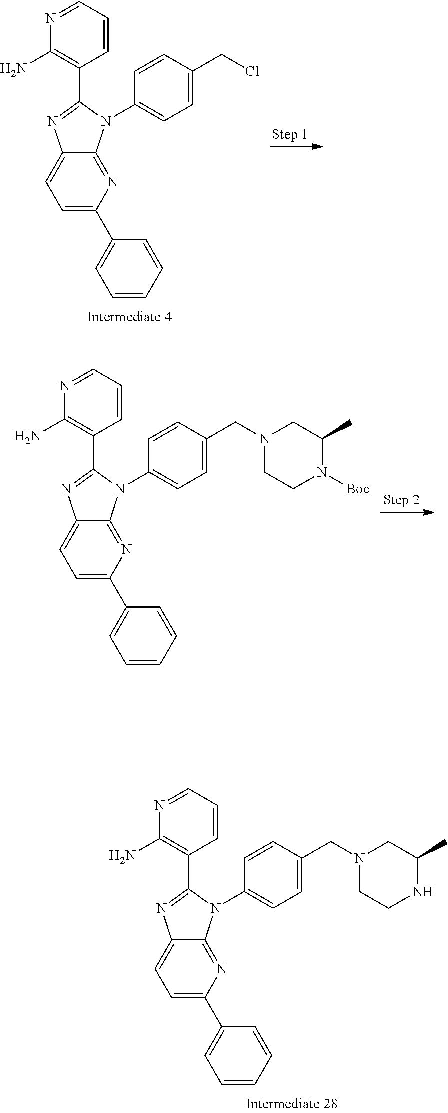 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)CCN1C(=O)OC(C)(C)C ; C[C@@H]1CN(Cc2ccc(-n3c(-c4cccnc4N)nc4ccc(-c5ccccc5)nc43)cc2)CCN1]

Intermediate 28 was prepared in a manner similar to Intermediate 25. MS: m/z=476.2 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (d, J=6.0 Hz, 1H), 7.04 (br s, 2H), 6.40-6.34 (m, 1H), 3.53 (s, 2H), 2.87-2.77 (m, 1H), 2.75-2.62 (m, 4H), 2.02-1.91 (m, 1H), 1.68-1.58 (m, 1H), 0.92 (d, J=6.0 Hz, 3H).

### Intermediate 29: 3-(3-(4-((3,8-diazabicyclo[3.2.1]octan-3-yl)methyl)phenyl)-5-phenyl-3H-imidazo[4,5-b]pyridin-2-yl)pyridin-2-amine

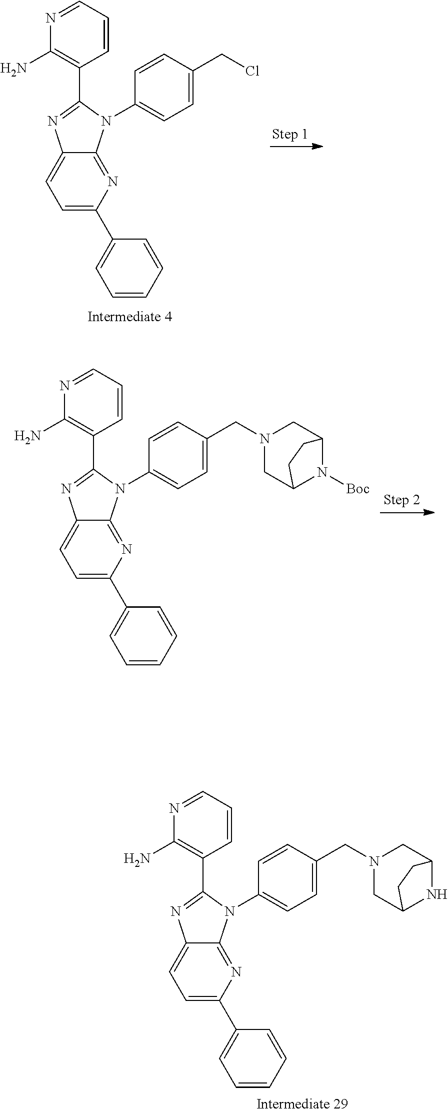 [SMILES: Nc1ncccc1-c1nc2ccc(-c3ccccc3)nc2n1-c1ccc(CCl)cc1 ; CC(C)(C)OC(=O)N1C2CCC1CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3CCC(C2)N3)cc1]

Intermediate 29 was prepared in a manner similar to Intermediate 25. MS: m/z=488.1 [M+H]⁺. ¹H NMR (400 MHz, Methanol-d₄) δ 8.34-8.28 (m, 1H), 8.05-7.95 (m, 4H), 7.90-7.85 (m, 3H), 7.65 (d, J=8.0 Hz, 2H), 7.48-7.37 (m, 3H), 6.90 (t, J=6.8 Hz, 1H), 4.35-4.25 (m, 4H), 3.50-3.40 (m, 4H), 2.41 (d, J=8.4 Hz, 2H), 2.25-2.16 (m, 2H).

### Intermediate 30: 3-(3-(4-((3,8-diazabicyclo[3.2.1]octan-8-yl)methyl)phenyl)-5-phenyl-3H-imidazo[4,5-b]pyridin-2-yl)pyridin-2-amine

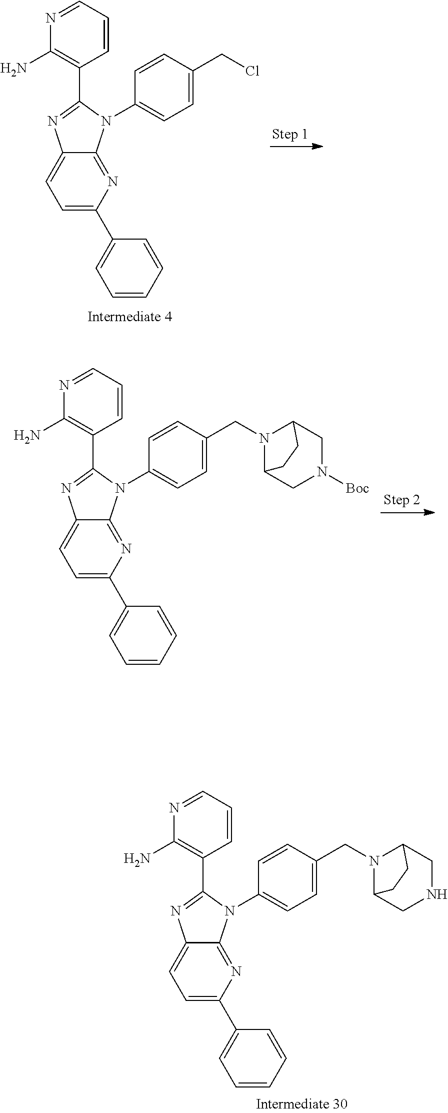 [SMILES: Nc1ncccc1-c1nc2ccc(-c3ccccc3)nc2n1-c1ccc(CCl)cc1 ; CC(C)(C)OC(=O)N1CC2CCC(C1)N2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C3CCC2CNC3)cc1]

Intermediate 30 was prepared in a manner similar to Intermediate 25. MS: m/z=488.2 [M+H]⁺. ¹H NMR (400 MHz, Dimethylsulfoxide-d₆) δ 12.42 (br s, 1H), 10.4-10.0 (m, 2H), 8.55 (br s, 1H), 8.38 (d, J=8.0 Hz, 1H), 8.13-8.07 (m, 1H), 8.06-8.00 (m, 5H), 7.98-7.94 (m, 1H), 7.69 (d, J=8.0 Hz, 2H), 7.50-7.40 (m, 3H), 6.94 (t, J=7.2 Hz, 1H), 4.39-4.33 (m, 2H), 4.01 (s, 2H), 3.95-3.89 (m, 3H), 3.42-3.39 (m, 3H), 2.47-2.38 (m, 2H).

### Intermediate 31: 1-(4-(2-(2-aminopyridin-3-yl)-5-phenyl-3H-imidazo[4,5-b]pyridin-3-yl)benzyl)azepan-4-amine

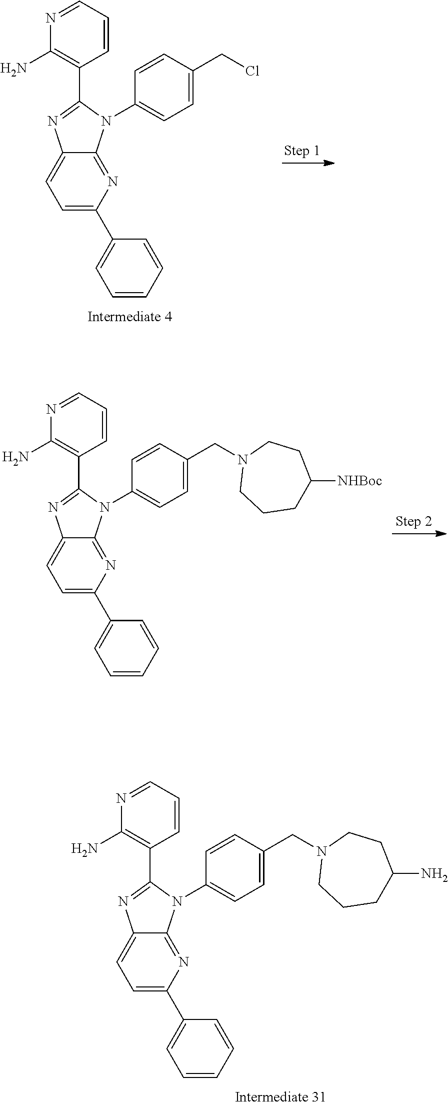 [SMILES: Nc1ncccc1-c1nc2ccc(-c3ccccc3)nc2n1-c1ccc(CCl)cc1 ; CC(C)(C)OC(=O)NC1CCCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCCC(N)CC2)cc1]

Intermediate 31 was prepared in a manner similar to Intermediate 27. MS: m/z=490.2 [M+H]⁺. ¹H NMR (400 MHz, Dimethylsulfoxide-d₆) δ 11.56-11.29 (m, 1H), 8.37 (J=8.4 Hz, 1H), 8.36-8.18 (m, 3H), 8.14 (J=7.6 Hz, 1H), 8.12-7.97 (m, 3H), 7.96-7.80 (m, 3H), 7.67 (d, J=8.4 Hz, 2H), 7.52-7.38 (m, 3H), 6.93-6.84 (m, 1H), 4.52-4.33 (m, 2H), 3.40-3.38 (m, 2H), 3.21-3.12 (m, 2H), 3.09-2.96 (m, 1H), 2.26-1.98 (m, 4H), 1.93-1.80 (m, 1H), 1.75-1.61 (m, 1H).

### Intermediate 32: 1-(4-(2-(2-aminopyridin-3-yl)-5-phenyl-3H-imidazo[4,5-b]pyridin-3-yl)benzyl)azepan-3-amine

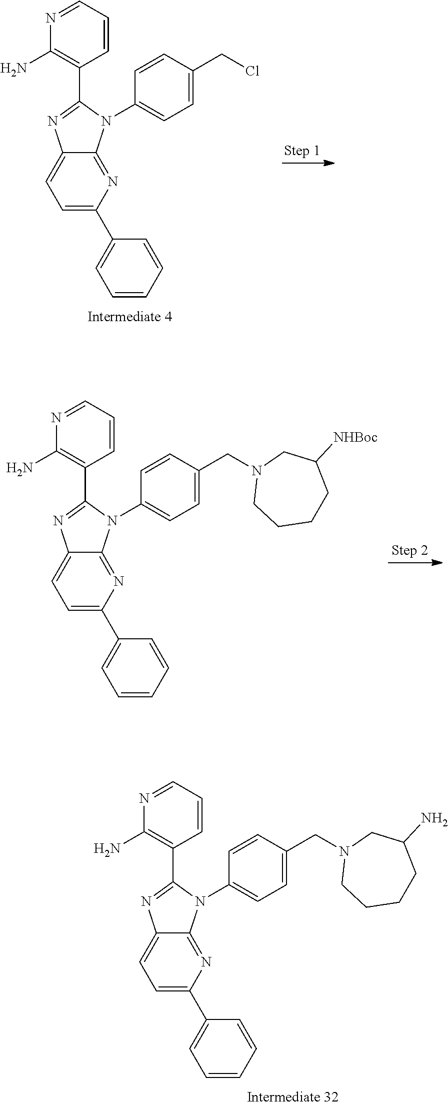 [SMILES: Nc1ncccc1-c1nc2ccc(-c3ccccc3)nc2n1-c1ccc(CCl)cc1 ; CC(C)(C)OC(=O)NC1CCCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCCCC(N)C2)cc1]

Intermediate 32 was prepared in a manner similar to Intermediate 18. MS: m/z=490.2 [M+H]⁺. ¹H NMR (400 MHz, Dimethylsulfoxide-d₆) δ 8.27 (d, J=8.4 Hz, 1H), 8.05-7.98 (m, 4H), 7.53-7.37 (m, 7H), 7.16-7.12 (m, 1H), 7.07 (br s, 2H), 6.38 (dd, J=7.6, 4.8 Hz, 1H), 3.77-3.72 (m, 2H), 3.62-3.53 (m, 1H), 2.89-2.78 (m, 2H), 2.65-2.61 (m, 2H), 2.59-2.53 (m, 2H), 1.82-1.73 (m, 1H), 1.67-1.48 (m, 4H), 1.46-1.25 (m, 1H).

### Intermediate 33: (S)-3-(3-(4-((3-Methylpiperazin-1-yl)methyl)phenyl)-5-phenyl-3H-imidazo[4,5-b]pyridin-2-yl)pyridin-2-amine

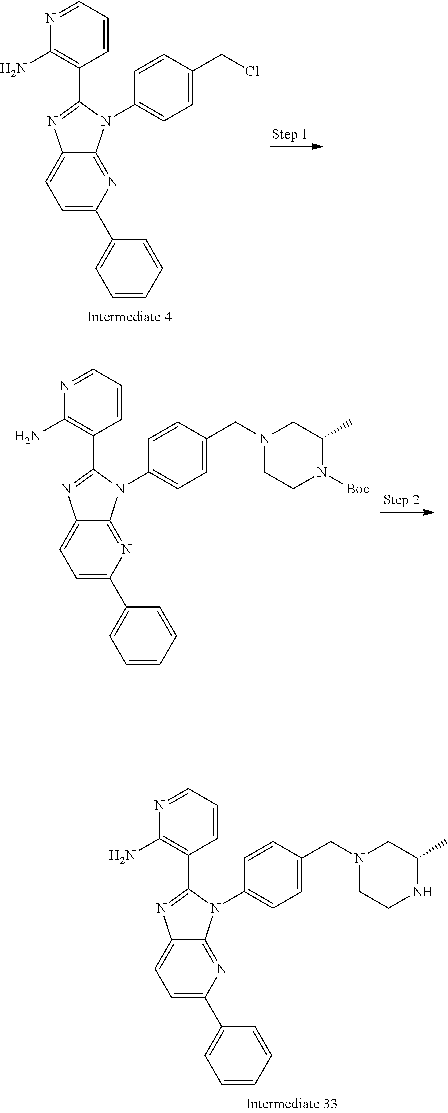 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)CCN1C(=O)OC(C)(C)C ; C[C@H]1CN(Cc2ccc(-n3c(-c4cccnc4N)nc4ccc(-c5ccccc5)nc43)cc2)CCN1]

Intermediate 33 was prepared in a manner similar to Intermediate 25. MS: m/z=476.2 [M+H]⁺. ¹H NMR (400 MHz, Methanol-d₄) δ 8.32 (d, J=8.4 Hz, 1H), 8.07-7.99 (m, 4H), 7.94-7.84 (m, 3H), 7.73 (d, J=8.4 Hz, 2H), 7.50-7.37 (m, 3H), 6.96-6.88 (m, 1H), 4.57 (s, 1H), 4.60-4.53 (m, 1H), 3.94-3.83 (m, 1H), 3.81-3.69 (m, 3H), 3.68-3.56 (m, 1H), 3.54-3.41 (m, 1H), 1.45 (d, J=6.8 Hz, 3H).

### Intermediate 34: 3-(3-(4-(4,7-Diazaspiro[2.5]octan-7-ylmethyl)phenyl)-5-phenyl-3H-imidazo[4,5-b]pyridin-2-yl)pyridin-2-amine

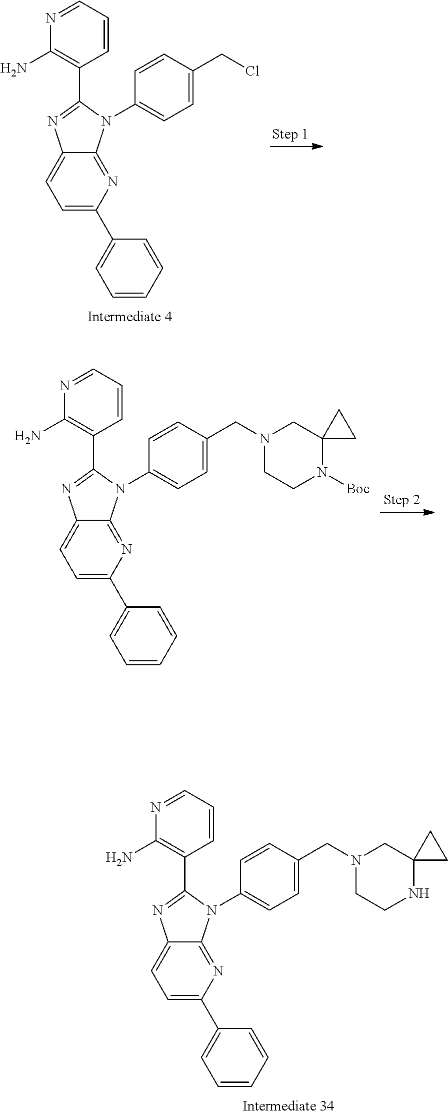 [SMILES: Nc1ncccc1-c1nc2ccc(-c3ccccc3)nc2n1-c1ccc(CCl)cc1 ; CC(C)(C)OC(=O)N1CCN(Cc2ccc(-n3c(-c4cccnc4N)nc4ccc(-c5ccccc5)nc43)cc2)CC12CC2 ; Nc1ncccc1-c1nc2ccc(-c3ccccc3)nc2n1-c1ccc(CN2CCNC3(CC3)C2)cc1]

Intermediate 34 was prepared in a manner similar to Intermediate 25. MS: m/z=488.3 [M+H]⁺. ¹H NMR (400 MHz, Dimethylsulfoxide-d₆) δ 8.26 (d, J=8.4 Hz, 1H), 8.09-7.92 (m, 4H), 7.56-7.33 (m, 7H), 7.14 (dd, J=7.8, 1.8 Hz, 1H), 7.05 (br s, 2H), 6.34 (dd, J=7.6, 4.8 Hz, 1H), 3.52 (s, 2H), 2.76 (t, J=4.8 Hz, 2H), 2.44-2.32 (m, 2H), 2.18 (s, 3H), 0.47-0.38 (m, 2H), 0.35-0.25 (m, 2H).

### Intermediate 35: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

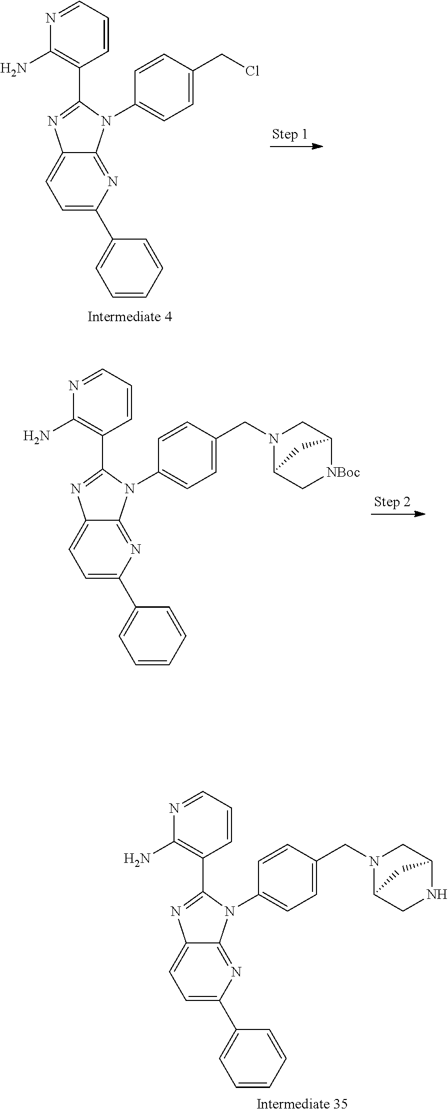 [SMILES: Nc1ncccc1-c1nc2ccc(-c3ccccc3)nc2n1-c1ccc(CCl)cc1 ; CC(C)(C)OC(=O)N1C[C@@H]2C[C@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@@H]3C[C@H]2CN3)cc1]

Intermediate 35 was prepared in a manner similar to Intermediate 27. MS: m/z=474.2 [M+H]⁺. ¹H NMR (400 MHz, Dimethylsulfoxide-d₆) δ 12.43-11.57 (m, 1H), 10.39-9.51 (m, 2H), 8.55-8.39 (m, 1H), 8.38 (d, J=8.4 Hz, 1H), 8.14 (d, J=5.2 Hz, 1H), 8.11-8.01 (m, 3H), 7.94 (d, J=8.0 Hz, 2H), 7.84 (d, J=7.2 Hz, 1H), 7.69 (d, J=8.4 Hz, 2H), 7.54-7.40 (m, 3H), 6.91-6.87 (m, 1H), 4.68-4.40 (m, 4H), 3.99-3.94 (m, 1H), 3.80-3.75 (m, 2H), 3.42-3.40 (m, 1H), 2.60-2.52 (m, 1H), 2.20-2.06 (m, 1H).

### Intermediate 36: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

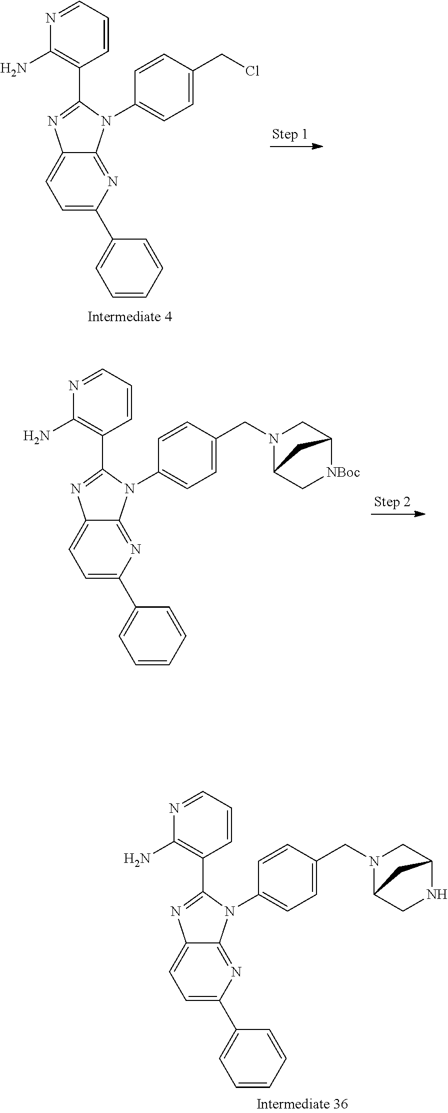 [SMILES: Nc1ncccc1-c1nc2ccc(-c3ccccc3)nc2n1-c1ccc(CCl)cc1 ; CC(C)(C)OC(=O)N1C[C@H]2C[C@@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@H]3C[C@@H]2CN3)cc1]

Intermediate 36 was prepared in a manner similar to Intermediate 25. MS: m/z=474.2 [M+H]⁺. ¹H NMR (400 MHz, Methanol-d₄) δ 8.33 (d, J=8.4 Hz, 1H), 8.06-8.00 (m, 6H), 7.88 (dd, J=7.6, 1.6 Hz, 1H), 7.73 (d, J=8.4 Hz, 2H), 7.46-7.40 (m, 3H), 6.92 (dd, J=7.6, 6.4 Hz, 1H), 4.84-4.81 (m, 1H), 4.75-4.65 (m, 2H), 4.14 (d, J=12.8 Hz, 1H), 3.96-3.92 (m, 1H), 3.74-3.71 (m, 1H), 3.62-3.60 (m, 1H), 3.38-3.32 (m, 1H), 2.87-2.82 (m, 1H), 2.39-2.36 (m, 1H).

### Intermediate 37: 3-(3-(4-(((3S,5R)-3,5-Dimethylpiperazin-1-yl)methyl)phenyl)-5-phenyl-3H-imidazo[4,5-b]pyridin-2-yl)pyridin-2-amine

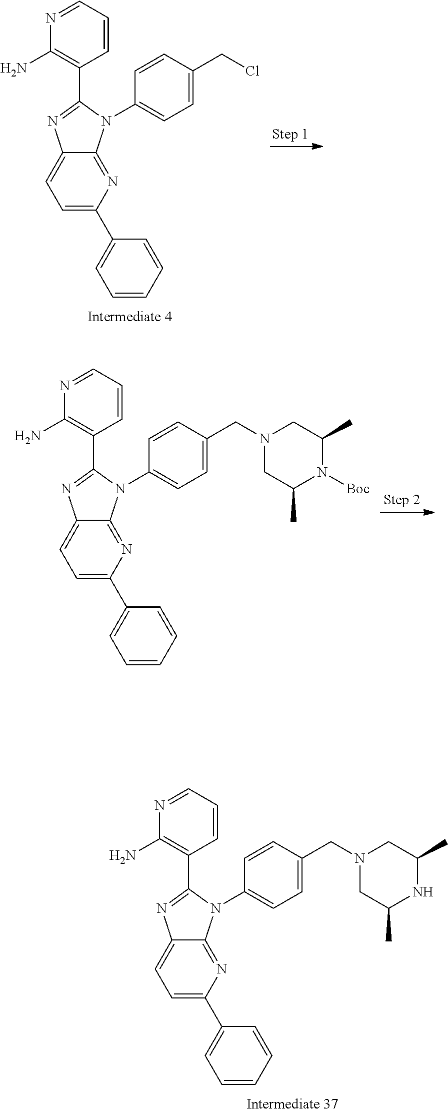 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)C[C@H](C)N1C(=O)OC(C)(C)C ; C[C@@H]1CN(Cc2ccc(-n3c(-c4cccnc4N)nc4ccc(-c5ccccc5)nc43)cc2)C[C@H](C)N1]

Intermediate 37 was prepared in a manner similar to Intermediate 25. MS: m/z=490.1 [M+H]⁺. ¹H NMR (400 MHz, Dimethylsulfoxide-d₆) δ 8.27 (d, J=8.4 Hz, 1H), 7.97-8.04 (m, 4H), 7.42-7.48 (m, 6H), 7.37-7.42 (m, 1H), 7.15 (dd, J=7.6, 2.0 Hz, 1H), 7.05 (br s, 2H), 6.36 (dd, J=7.6, 4.8 Hz, 1H), 3.52 (s, 2H), 2.73-2.79 (m, 2H), 2.64-2.67 (m, 2H), 1.56-1.50 (m, 2H), 0.91 (d, J=6.0 Hz, 6H).

### Intermediate 38: 3-(3-(4-((2,5-Diazabicyclo[2.2.2]octan-2-yl)methyl)phenyl)-5-phenyl-3H-imidazo[4,5-b]pyridin-2-yl)pyridin-2-amine

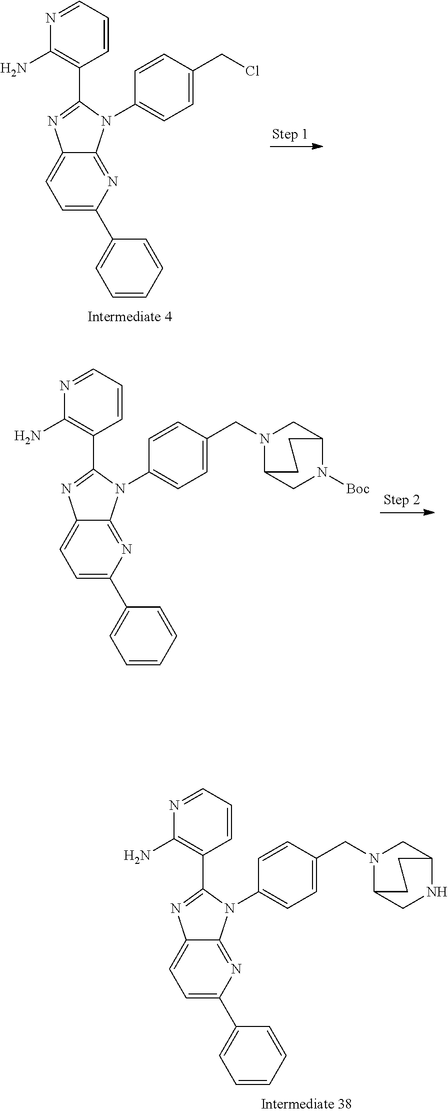 [SMILES: Nc1ncccc1-c1nc2ccc(-c3ccccc3)nc2n1-c1ccc(CCl)cc1 ; CC(C)(C)OC(=O)N1CC2CCC1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2CC3CCC2CN3)cc1]

Intermediate 38 was prepared in a manner similar to Intermediate 18. MS: m/z=488.2 [M+H]⁺. ¹H NMR (400 MHz, Dimethylsulfoxide-d₆) δ 12.11-11.44 (m, 1H), 10.27-9.61 (m, 2H), 8.56-8.40 (m, 1H), 8.38 (d, J=8.4 Hz, 1H), 8.15 (dd, J=6.0, 1.2 Hz, 1H), 8.10-7.99 (m, 5H), 7.86 (dd, J=7.2, 1.2 Hz, 1H), 7.68 (d, J=8.4 Hz, 2H), 7.51-7.46 (m, 2H), 7.46-7.40 (m, 1H), 6.91 (dd, J=7.6, 6.4 Hz, 1H), 4.62 (br s, 2H), 4.01-3.79 (m, 4H), 3.76-3.70 (m, 2H), 2.21 (m, 2H), 1.97-1.80 (m, 2H).

### Intermediate 39: 3-(3-(4-((2,6-diazaspiro[3.4]octan-2-yl)methyl)phenyl)-5-phenyl-3H-imidazo[4,5-b]pyridin-2-yl)pyridin-2-amine

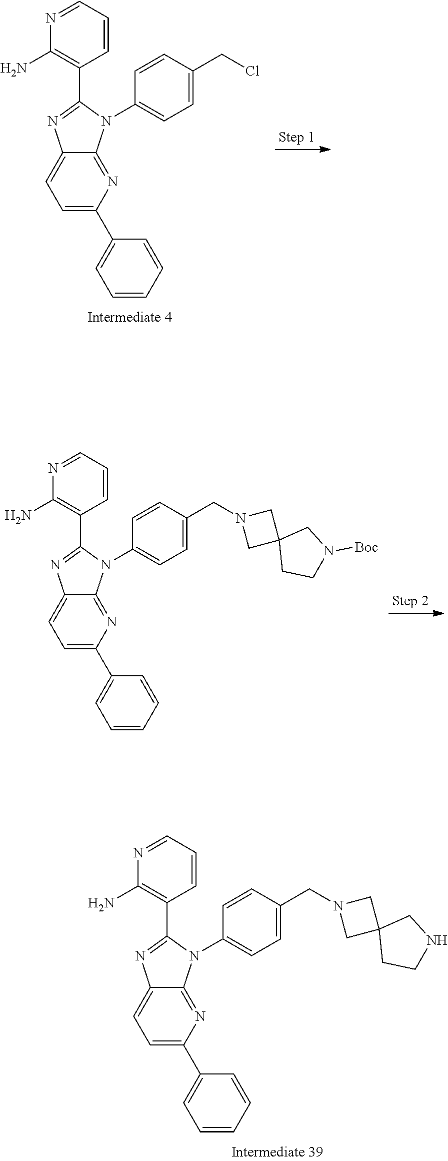 [SMILES: Nc1ncccc1-c1nc2ccc(-c3ccccc3)nc2n1-c1ccc(CCl)cc1 ; CC(C)(C)OC(=O)N1CCC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CCNC3)C2)cc1]

Intermediate 39 was prepared in a manner similar to Intermediate 5. MS: m/z=488.3 [M+H]⁺. ¹H NMR (400 MHz, Dimethylsulfoxide-d₆) δ 8.26 (d, J=8.4 Hz, 1H), 8.06-7.95 (m, 4H), 7.51-7.36 (m, 7H), 7.15 (dd, J=7.6, 1.6 Hz, 1H), 6.99 (br s, 2H), 6.39 (dd, J=7.6, 4.8 Hz, 1H), 3.63 (s, 2H), 3.16-3.10 (m, 4H), 2.88 (s, 2H), 2.74 (t, J=6.8 Hz, 2H), 1.82 (t, J=6.8 Hz, 2H), 1.25-1.22 (m, 1H).

### Intermediate 40: 3-(3-(4-((2,6-Diazaspiro[3.5]nonan-2-yl)methyl)phenyl)-5-phenyl-3H-imidazo[4,5-b]pyridin-2-yl)pyridin-2-amine

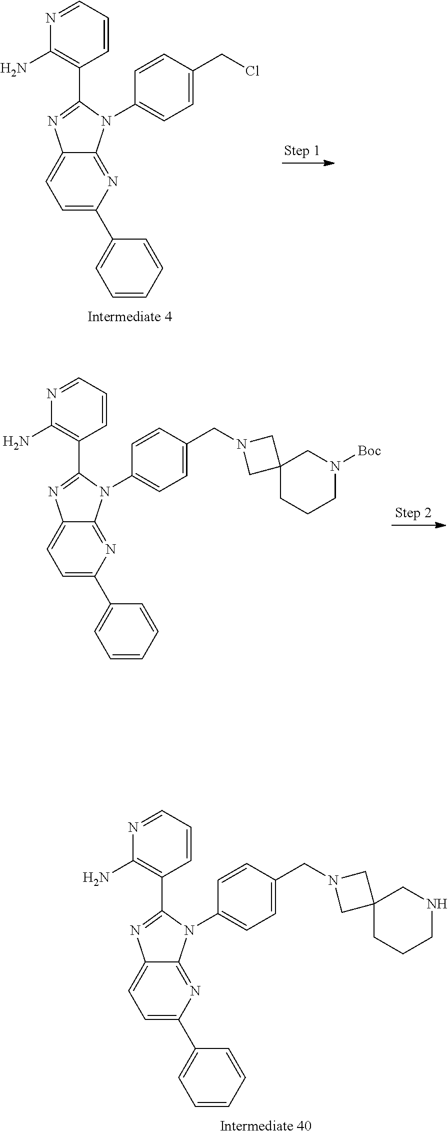 [SMILES: Nc1ncccc1-c1nc2ccc(-c3ccccc3)nc2n1-c1ccc(CCl)cc1 ; CC(C)(C)OC(=O)N1CCCC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CCCNC3)C2)cc1]

Intermediate 40 was prepared in a manner similar to Intermediate 18. MS: m/z=502.3 [M+H]⁺. ¹H NMR (400 MHz, Dimethylsulfoxide-d₆) δ 8.26 (d, J=8.4 Hz, 1H), 8.03-7.97 (m, 5H), 7.50-7.39 (m, 6H), 7.15 (dd, J=7.6, 1.6 Hz, 1H), 7.01 (br s, 2H), 6.36 (dd, J=7.6, 4.8 Hz, 1H), 3.66 (s, 2H), 3.04 (d, J=7.2 Hz, 2H), 2.85 (d, J=6.8 Hz, 2H), 2.78 (s, 2H), 2.62-2.58 (m, 2H), 1.64-1.60 (m, 2H), 1.40-1.35 (m, 2H).

### Intermediate 41: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

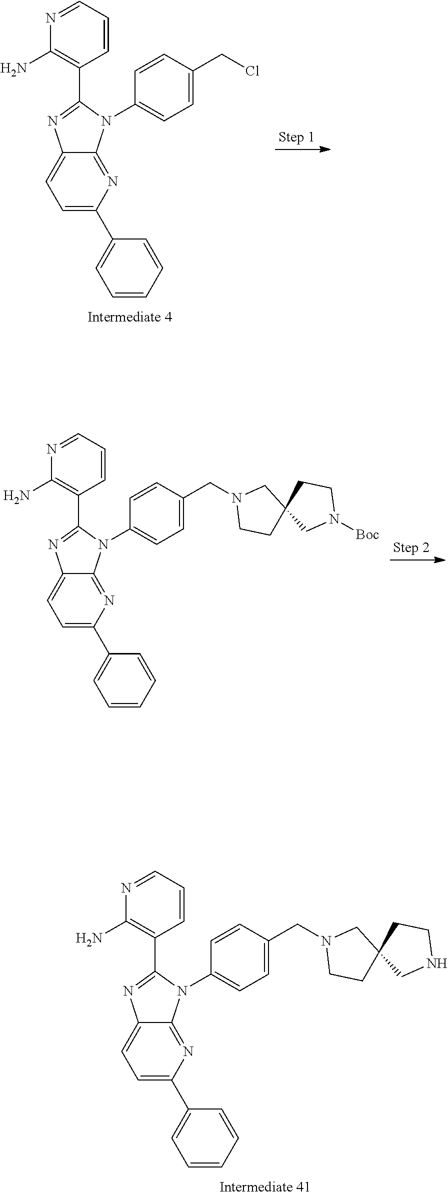 [SMILES: Nc1ncccc1-c1nc2ccc(-c3ccccc3)nc2n1-c1ccc(CCl)cc1 ; CC(C)(C)OC(=O)N1CC[C@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@]3(CCNC3)C2)cc1]

Intermediate 41 was prepared in a manner similar to Intermediate 25. MS: m/z=502.3 [M+H]⁺. ¹H NMR (400 MHz, Dimethylsulfoxide-d₆) δ 11.9 (br s, 1H), 9.80-9.66 (m, 2H), 8.66-8.50 (m, 2H), 8.38 (d, J=8.4 Hz, 1H), 8.17 (dd, J=6.4, 1.6 Hz, 1H), 8.08-8.05 (m, 3H), 7.93-7.86 (M, 3H), 7.66 (d, J=8.4 Hz, 2H), 7.54-7.40 (m, 3H), 7.00-6.95 (m, 1H), 3.55-3.50 (m, 1H), 3.40-3.17 (m, 8H), 2.33-1.95 (m, 5H).

### Intermediate 42: 3-(3-(4-((4-((Methyl-d3)amino)piperidin-1-yl)methyl)phenyl)-5-phenyl-3H-imidazo[4,5-b]pyridin-2-yl)pyridin-2-amine

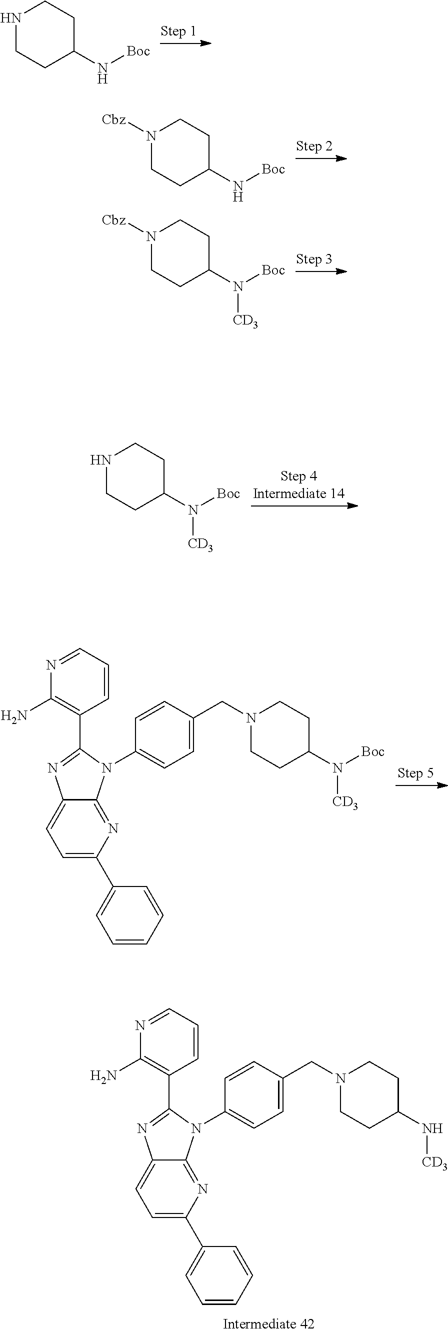

### Step 1: Benzyl 4-((tert-butoxycarbonyl)amino)piperidine-1-carboxylate

To a solution of tert-butyl N-(4-piperidyl)carbamate (12 g, 59.9 mmol) in CH₂Cl₂ (100 mL) were added TEA (18.2 g, 179 mmol). Then CbzCl (11.2 g, 65.9 mmol) was added into the mixture at 0° C. The mixture was stirred at 25° C. for 2 hr. The mixture was concentrated under reduced pressure. The crude was purified by silica gel flash chromatography (Eluent of 10~50% EtOAc in petroleum ether) to give benzyl 4-((tert-butoxycarbonyl)amino)piperidine-1-carboxylate (16 g, yield: 71%) as an off-white solid. ¹H NMR (400 MHz, Dimethylsulfoxide-d₆) δ 7.40-7.28 (m, 5H), 5.06 (s, 2H), 3.90 (d, J=13.6 Hz, 2H), 3.50-3.34 (m, 2H), 2.89 (s, 2H), 1.71 (d, J=10.8 Hz, 2H), 1.37 (s, 9H), 1.30-1.18 (m, 2H).

### Step 2: Benzyl 4-((tert-butoxycarbonyl)(methyl-d₃)amino)piperidine-1-carboxylate

To a solution of benzyl 4-(tert-butoxycarbonylamino)piperidine-1-carboxylate (13 g, 38.9 mmol) in THF (200 mL) was added NaH (4.66 g, 117 mmol) at 0° C. After stirring at 0° C. for 30 min, CD₃I (16.5 g, 117 mmol) was added to the mixture. The mixture was stirred at 25° C. for 16 hr. The mixture was quenched with NH₄Cl (aq) (100 mL) at 0° C. The mixture was duilted with H₂O (100 mL) and extrated with CH₂Cl₂ (200 mL×2). The combined organic layers were washed with brine (200 mL×2), dried over Na₂SO₄, filtered and concentrated under reduced pressure. The residue was purified by silica gel flash chromatography (Eluent of 0~20% EtOAc in petroleum ether) to give benzyl 4-((tert-butoxycarbonyl)(methyl-d₃)amino)piperidine-1-carboxylate (8.6 g, yield: 56%) as a colorless oil. MS: m/z=252.3 [M+H−100]⁺. ¹H NMR (400 MHz, Dimethylsulfoxide-d₆) δ 7.42-7.28 (m, 5H), 5.07 (s, 2H), 4.14-3.89 (m, 3H), 2.82 (s, 2H), 1.58-1.48 (m, 4H), 1.39 (s, 9H).

### Step 3: tert-Butyl (methyl-d₃)(piperidin-4-yl)carbamate

To a solution of benzyl 4-[tert-butoxycarbonyl(trideuteriomethyl)amino]piperidine-1-carboxylate (8.6 g, 24.5 mmol) in MeOH (90 mL) was added Pd/C (900 mg, 24.5 mmol). The mixture was stirred at 25° C. for 16 hr under H₂ (15 psi). The mixture was filtered, and the filter cake was washed with MeOH (30 mL). The filtrate was concentrated under reduced pressure to give a tert-butyl (methyl-d₃)(piperidin-4-yl)carbamate (5 g, yield: 80%) as a colorless oil. MS: m/z=218.3 [M+H]⁺. ¹H NMR (400 MHz, Dimethylsulfoxide-d₆) δ 3.93-3.62 (m, 1H), 3.26-3.16 (m, 1H), 2.95 (d, J=12.0 Hz, 2H), 2.47-2.38 (m, 2H), 1.54-1.42 (m, 4H), 1.39 (s, 9H).

### Step 4: tert-Butyl (1-(4-(2-(2-aminopyridin-3-yl)-5-phenyl-3H-imidazo[4,5-b]pyridin-3-yl)benzyl)piperidin-4-yl)(methyl-d₃)carbamate

To a solution of Intermediate 4 (1 g, 2.43 mmol) in DMF (10 mL) were added tert-butyl (methyl-d₃)(piperidin-4-yl)carbamate (527 mg, 2.43 mmol), NaI (182 mg, 1.21 mmol) and K₂CO₃ (1.0 g, 7.28 mmol). The mixture was stirred at 80° C. for 18 hr. The reaction mixture was diluted with H₂O (20 mL) and extracted with CH₂Cl₂ (20 mL×3). The combined organic layers were washed with brine (20 mL×2), dried over Na₂SO₄, filtered and concentrated under reduced pressure. The residue was purified by silica gel flash chromatography (Eluent of 0~6% MeOH in CH₂Cl₂) to give tert-butyl (1-(4-(2-(2-aminopyridin-3-yl)-5-phenyl-3H-imidazo[4,5-b]pyridin-3-yl)benzyl)piperidin-4-yl)(methyl-d₃)carbamate (750 mg, yield: 49%) as a yellow solid. MS: m/z=593.3 [M+H]⁺.

### Step 5: 3-(3-(4-((4-((Methyl-d₃)amino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(methyl-d₃)carbamate (430 mg, 725 μmol) in CH₂Cl₂ (5 mL) was added TFA (165 mg, 1.45 mmol). The mixture was stirred at 25° C. for 1 hr. The mixture was diluted with H₂O (10 mL). The pH of the mixture was adjusted to about 8 with NaHCO₃ (aq.). The mixture was extracted with CH₂Cl₂ (10 mL×2). The combined organic layers were washed with brine (10 mL×2), dried over Na₂SO₄, filtered, and concentrated under reduced pressure. After purification by prep-TLC (CH₂Cl₂: MeOH=5:1), 3-(3-(4-((4-((methyl-d₃)amino)piperidin-1-yl)methyl)phenyl)-5-phenyl-3H-imidazo[4,5-b]pyridin-2-yl)pyridin-2-amine (Intermediate 42, 350 mg crude, yield: 50%) was obtained as a light-yellow solid. MS: m/z=493.3 [M+H]⁺. ¹H NMR (400 MHz, Methanol-d₄) δ 8.19 (d, J=8.4 Hz, 1H), 8.03 (d, J=7.2 Hz, 2H), 7.98 (dd, J=4.8, 1.6 Hz, 1H), 7.93 (d, J=8.4 Hz, 1H), 7.53 (d, J=8.4 Hz, 2H), 7.47-7.35 (m, 5H), 7.32 (dd, J=7.6, 1.6 Hz, 1H), 6.47 (dd, J=7.6, 4.8 Hz, 1H), 3.65 (s, 2H), 3.08-3.03 (m, 2H), 3.02-2.95 (m, 1H), 2.21-2.15 (m, 2H), 2.08-2.05 (m, 2H), 1.67-1.61 (m, 2H).

### Intermediate 43: 4-(4-(2-(2-Aminopyridin-3-yl)-5-phenyl-3H-imidazo[4,5-b]pyridin-3-yl)benzyl)piperazin-1-amine hydrochloride

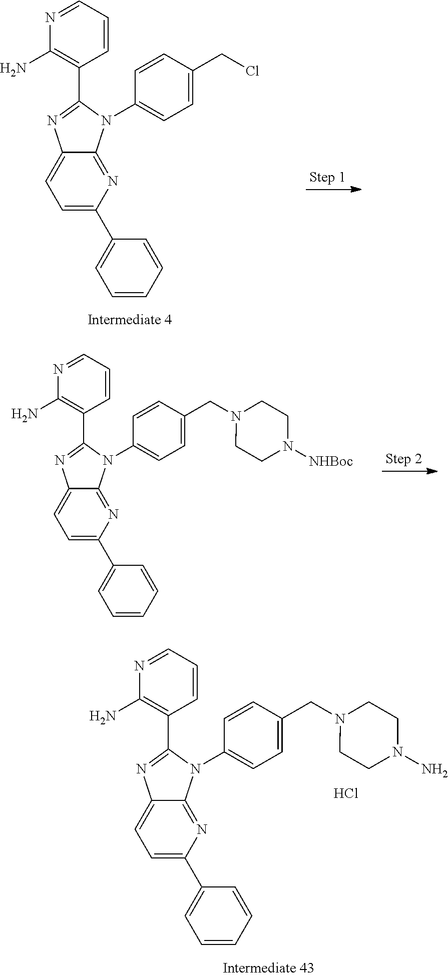

Intermediate 43 was prepared in a manner similar to Intermediate 18. MS: m/z=477.3 [M+H]⁺.

### Intermediate 44: 3-(3-(4-(((3aR,6aS)-hexahydropyrrolo[3,4-c]pyrrol-2(1H)-yl)methyl)phenyl)-5-phenyl-3H-imidazo[4,5-b]pyridin-2-yl)pyridin-2-amine

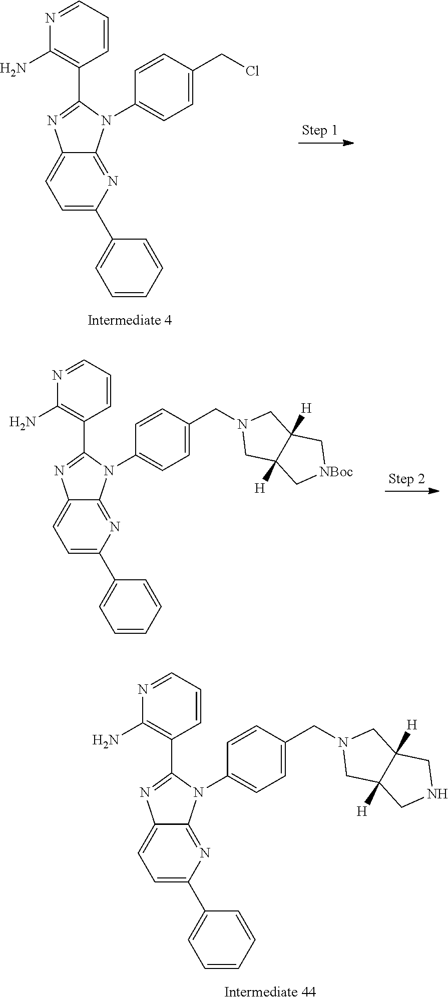 [SMILES: Nc1ncccc1-c1nc2ccc(-c3ccccc3)nc2n1-c1ccc(CCl)cc1 ; CC(C)(C)OC(=O)N1C[C@H]2CN(Cc3ccc(-n4c(-c5cccnc5N)nc5ccc(-c6ccccc6)nc54)cc3)C[C@H]2C1 ; Nc1ncccc1-c1nc2ccc(-c3ccccc3)nc2n1-c1ccc(CN2C[C@H]3CNC[C@H]3C2)cc1]

Intermediate 44 was prepared in a manner similar to Intermediate 18. MS: m/z=488.2 [M+H]⁺. ¹H NMR (400 MHz, Dimethylsulfoxide-d₆) 8.27 (d, J=8.0 Hz, 1H), 8.04-7.97 (m, 4H), 7.49-7.44 (m, 5H), 7.43-7.37 (m, 2H), 7.16 (dd, J=7.6, 1.6 Hz, 1H), 7.04 (br s, 2H), 6.38 (dd, J=7.6, 4.8 Hz, 1H), 3.60 (s, 2H), 2.85-2.76 (m, 2H), 2.58-2.51 (m, 7H), 2.31-2.26 (m, 2H).

### Example 1: N-(1-(4-(2-(2-Aminopyridin-3-yl)-3H-imidazo[4,5-b]pyridin-3-yl)benzyl)piperidin-4-yl)cyanamide

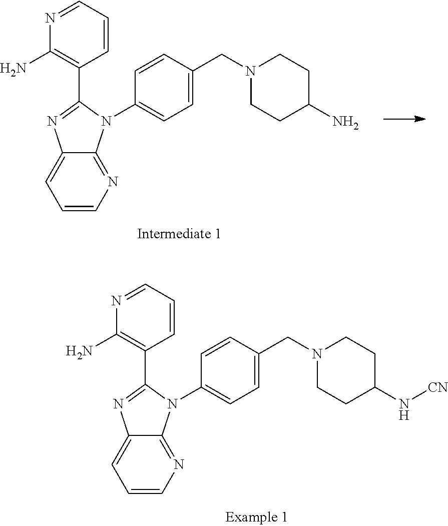 [SMILES: Nc1ncccc1-c1nc2cccnc2n1-c1ccc(CN2CCC(N)CC2)cc1 ; N#CNC1CCN(Cc2ccc(-n3c(-c4cccnc4N)nc4cccnc43)cc2)CC1]

To a solution of Intermediate 1 (200 mg, 500 mmol) in MeOH (2 mL) were added BrCN (160 mg, 1.51 mmol) and AcONa (65.7 mg, 801 mmol). The mixture was stirred at 25° C. for 4 hr. The mixture was diluted with H₂O (5 mL). The pH of mixture was adjusted to about 8 with NaHCO₃ (aq). The mixture was extracted with CH₂Cl₂ (10 mL×3) and washed with brine (3 mL), dried over Na₂SO₄, filtered, and concentrated under reduced pressure. The residue was purified byprep-HPLC (column: Phenomenex C18 150*25 mm 10 mm; mobile phase: [water (NH₄HCO₃)-ACN]; B %: 20%-50%, 8 min) to give N-(1-(4-(2-(2-aminopyridin-3-yl)-3H-imidazo[4,5-b]pyridin-3-yl)benzyl)piperidin-4-yl)cyanamide (Example 1, 10.1 mg, yield: 4.5%) as a light-yellow lyophilized powder. MS: m/z=425.1 [M+H]⁺. ¹H NMR (400 MHz, Dimethylsulfoxide-d₆) δ 8.33 (dd, J=4.8, 1.2 Hz, 1H), 8.20 (dd, J=8.0, 1.2 Hz, 1H), 7.99 (dd, J=4.8, 1.6 Hz, 1H), 7.45-7.35 (m, 5H), 7.16 (dd, J=7.6, 1.6 Hz, 1H), 7.00 (br s, 2H), 6.87 (br s, 1H), 6.37 (dd, J=7.6, 4.8 Hz, 1H), 3.53 (s, 2H), 3.08-2.99 (m, 1H), 2.79-2.71 (m, 2H), 2.13-2.05 (m, 2H), 1.84-1.77 (m, 2H), 1.49-1.39 (m, 2H).

### Example 2: 4-(4-(2-(2-Aminopyridin-3-yl)-5-phenyl-3H-imidazo[4,5-b]pyridin-3-yl)benzyl)piperazine-1-carbonitrile

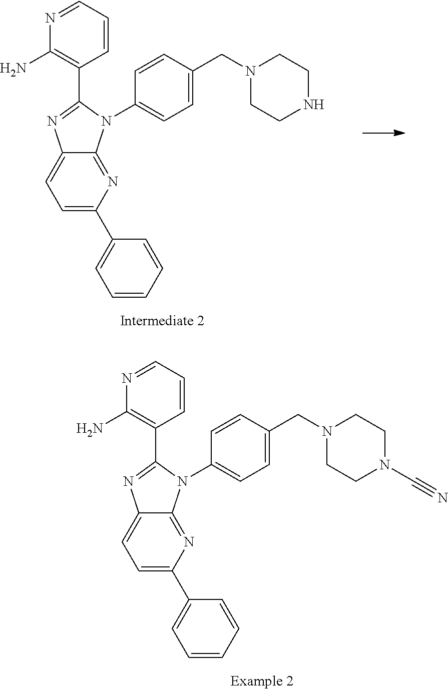 [SMILES: Nc1ncccc1-c1nc2ccc(-c3ccccc3)nc2n1-c1ccc(CN2CCNCC2)cc1 ; N#CN1CCN(Cc2ccc(-n3c(-c4cccnc4N)nc4ccc(-c5ccccc5)nc43)cc2)CC1]

Example 2 was prepared in a manner similar to Example 1. MS: m/z=487.1 [M+H]⁺. ¹H NMR (400 MHz, Dimethylsulfoxide-d₆) 8.27 (d, J=8.4 Hz, 1H), 8.06-7.95 (m, 4H), 7.52-7.42 (m, 6H), 7.42-7.37 (m, 1H), 7.15 (dd, J=7.6, 1.6 Hz, 1H), 7.02 (br s, 2H), 6.39 (dd, J=7.6, 4.8 Hz, 1H), 3.63 (s, 2H), 3.24 (t, J=4.8 Hz, 4H), 2.49-2.44 (m, 4H).

### Example 3: N-(1-(4-(2-(2-Aminopyridin-3-yl)-5-phenyl-3H-imidazo[4,5-b]pyridin-3-yl)benzyl)piperidin-4-yl)cyanamide

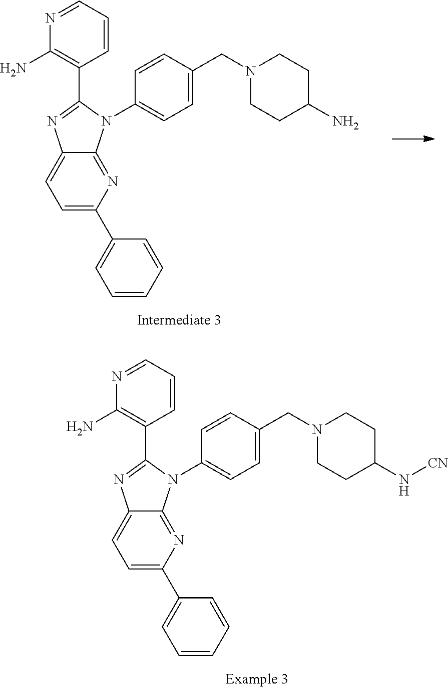 [SMILES: Nc1ncccc1-c1nc2ccc(-c3ccccc3)nc2n1-c1ccc(CN2CCC(N)CC2)cc1 ; N#CNC1CCN(Cc2ccc(-n3c(-c4cccnc4N)nc4ccc(-c5ccccc5)nc43)cc2)CC1]

Example 3 was prepared in a manner similar to Example 1. MS: m/z=501.3 [M+H]⁺. ¹H NMR (400 MHz, Dimethylsulfoxide-d₆) δ 8.27 (d, J=8.4 Hz, 1H), 8.05-7.97 (m, 4H), 7.49-7.45 (m, 6H), 7.37 (m, 1H), 7.16 (dd, J=7.6, 1.6 Hz, 1H), 7.02 (br s, 2H), 6.87 (br s, 1H), 6.38 (dd, J=4.8, 7.6 Hz, 1H), 3.56 (s, 2H), 3.11-2.90 (m, 1H), 2.79-2.73 (m, 2H), 2.13-2.06 (m, 2H), 1.86-1.78 (m, 2H), 1.52-1.41 (m, 2H).

### Example 4: N-(3-(2-(2-Aminopyridin-3-yl)-3-(4-((4-cyanamidopiperidin-1-yl)methyl)phenyl)-3H-imidazo[4,5-b]pyridin-5-yl)phenyl)acetamide

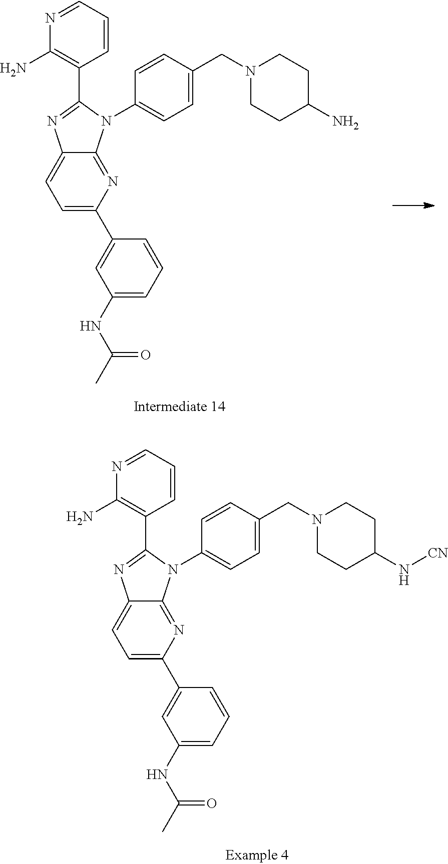 [SMILES: CC(=O)Nc1cccc(-c2ccc3nc(-c4cccnc4N)n(-c4ccc(CN5CCC(N)CC5)cc4)c3n2)c1 ; CC(=O)Nc1cccc(-c2ccc3nc(-c4cccnc4N)n(-c4ccc(CN5CCC(NC#N)CC5)cc4)c3n2)c1]

Following the general procedure of Example 1, the reaction of Intermediate 14 (300 mg, 563 mmol) with BrCN (140 mg, 1.32 mmol) was carried out. After purification by prep-HPLC (column: Waters xbridge 150*25 mm 10 mm; mobile phase: [water (NH₄HCO₃)-ACN]; B %: 13%-43%, 9 min), N-(3-(2-(2-aminopyridin-3-yl)-3-(4-((4-cyanamidopiperidin-1-yl)methyl)phenyl)-3H-imidazo[4,5-b]pyridin-5-yl)phenyl)acetamide (Example 4, 23.2 mg, yield: 7.1%) was obtained as a light-yellow lyophilized powder. MS: m/z=558.1 [M+H]⁺. ¹H NMR (400 MHz, Dimethylsulfoxide-d₆) δ 10.04 (s, 1H), 8.27 (d, J=8.4 Hz, 1H), 8.16 (s, 1H), 7.99 (dd, J=4.8, 1.6 Hz, 1H), 7.86 (d, J=8.4 Hz, 1H), 7.69-7.63 (m, 2H), 7.50-7.41 (m, 4H), 7.40-7.35 (m, 1H), 7.15 (dd, J=7.6, 1.6 Hz, 1H), 6.98 (br s, 2H), 6.87 (br s, 1H), 6.38 (dd, J=7.6, 4.8 Hz, 1H), 3.56 (s, 2H), 3.11-2.95 (m, 1H), 2.82-2.73 (m, 2H), 2.15-2.07 (m, 2H), 2.05 (s, 3H), 1.86-1.77 (m, 2H), 1.53-1.42 (m, 2H).

### Example 5: 6-(4-(2-(2-Aminopyridin-3-yl)-5-phenyl-3H-imidazo[4,5-b]pyridin-3-yl)benzyl)-2,6-diazaspiro[3.3]heptane-2-carbonitrile

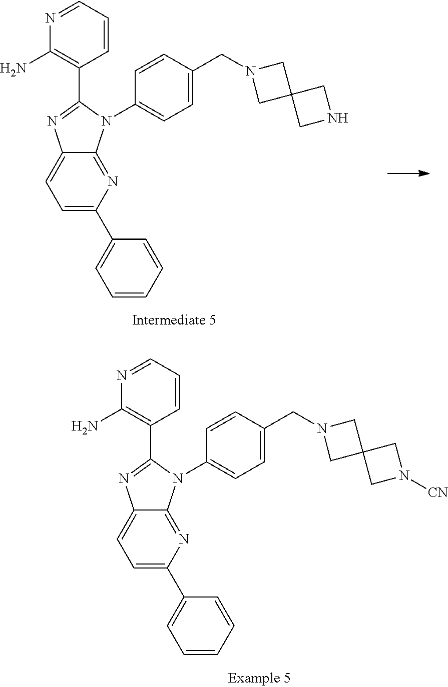 [SMILES: Nc1ncccc1-c1nc2ccc(-c3ccccc3)nc2n1-c1ccc(CN2CC3(CNC3)C2)cc1 ; N#CN1CC2(C1)CN(Cc1ccc(-n3c(-c4cccnc4N)nc4ccc(-c5ccccc5)nc43)cc1)C2]

To a solution of Intermediate 5 (50 mg, 105 μmol) in THF (2 mL) were added TEA (32 mg, 316 μmol) and BrCN (33.5 mg, 315 μmol). The mixture was stirred at 25° C. for 2 hr. The mixture was diluted with water (5 mL). The pH of the mixture was adjusted to 8 by addition of NaHCO₃ (aq) at 25° C. The mixture was extracted with CH₂Cl₂ (50 mL×2). The combined organic layers were washed with brine (50 mL), dried over Na₂SO₄, filtered, and concentrated under reduced pressure. The residue was purified by prep-HPLC (column: Waters xbridge 150*25 mm 10 μm; mobile phase: [water (NH₄HCO₃)-ACN]; B %: 32%-62%, 2 min) to give 6-(4-(2-(2-aminopyridin-3-yl)-5-phenyl-3H-imidazo[4,5-b]pyridin-3-yl)benzyl)-2,6-diazaspiro[3.3]heptane-2-carbonitrile (Example 5, 13.1 mg, yield: 24%) as a yellow solid. MS: m/z=499.2 [M+H]⁺. ¹H NMR (400 MHz, Methanol-d₄) δ 8.19 (d, J=8.4 Hz, 1H), 8.03 (d, J=7.6 Hz, 2H), 8.00-7.92 (m, 2H), 7.47-7.36 (m, 7H), 7.33-7.27 (m, 1H), 6.48 (dd, J=5.6, 7.6 Hz, 1H), 4.28 (s, 4H), 3.68 (s, 2H), 3.46 (s, 4H).

### Example 6: (S)—N-(1-(4-(2-(2-Aminopyridin-3-yl)-5-phenyl-3H-imidazo[4,5-b]pyridin-3-yl)benzyl)piperidin-3-yl)cyanamide

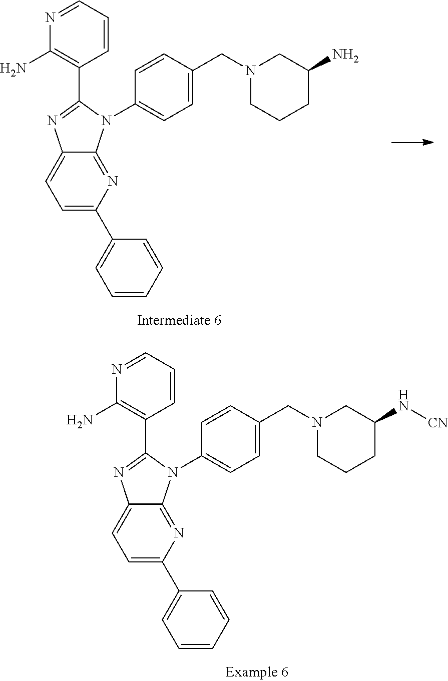 [SMILES: Nc1ncccc1-c1nc2ccc(-c3ccccc3)nc2n1-c1ccc(CN2CCC[C@H](N)C2)cc1 ; N#CN[C@H]1CCCN(Cc2ccc(-n3c(-c4cccnc4N)nc4ccc(-c5ccccc5)nc43)cc2)C1]

Example 6 was prepared in a manner similar to Example 5. MS: m/z=501.3 [M+H]⁺. ¹H NMR (400 MHz, Methanol-d₄) δ 8.19 (d, J=8.4 Hz, 1H), 8.06-8.02 (m, 2H), 7.98 (dd, J=5.2, 1.6 Hz, 1H), 7.94 (d, J=8.4 Hz, 1H), 7.54 (d, J=8.4 Hz, 2H), 7.47-7.41 (m, 4H), 7.40-7.35 (m, 1H), 7.31 (dd, J=7.6, 1.6 Hz, 1H), 6.50 (dd, J=7.6, 4.8 Hz, 1H), 3.69-3.61 (m, 2H), 3.35 (s, 2H), 3.27-3.18 (m, 1H), 2.92-2.84 (m, 1H), 2.74-2.64 (m, 1H), 2.21-2.15 (m, 1H), 1.95-1.89 (m, 1H), 1.85-1.77 (m, 1H), 1.66-1.60 (m, 1H).

### Example 7: (S)—N-(1-(4-(2-(2-Aminopyridin-3-yl)-5-phenyl-3H-imidazo[4,5-b]pyridin-3-yl)benzyl)pyrrolidin-3-yl)cyanamide

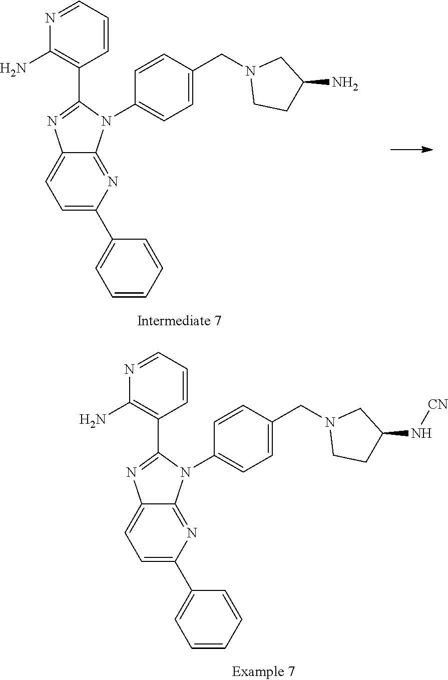 [SMILES: Nc1ncccc1-c1nc2ccc(-c3ccccc3)nc2n1-c1ccc(CN2CC[C@H](N)C2)cc1 ; N#CN[C@H]1CCN(Cc2ccc(-n3c(-c4cccnc4N)nc4ccc(-c5ccccc5)nc43)cc2)C1]

Example 7 was prepared in a manner similar to Example 5. MS: m/z=487.2 [M+H]⁺. ¹H NMR (400 MHz, Methanol-d₄) δ 8.20 (d, J=8.4 Hz, 1H), 8.04 (d, J=7.2 Hz, 2H), 8.00-7.97 (m, 1H), 7.95 (d, J=8.4 Hz, 1H), 7.56 (d, J=8.4 Hz, 2H), 7.47-7.40 (m, 4H), 7.38-7.33 (m, 2H), 6.57-6.41 (m, 1H), 4.58 (br s, 2H), 3.84-3.76 (m, 2H), 3.75-3.69 (m, 1H), 2.83-2.79 (m, 1H), 2.61-2.54 (m, 1H), 2.34-2.21 (m, 1H), 1.89-1.74 (m, 1H).

### Example 8: N-(2-(4-(2-(2-Aminopyridin-3-yl)-5-phenyl-3H-imidazo[4,5-b]pyridin-3-yl)benzyl)-2-azaspiro[4.5]decan-8-yl)cyanamide

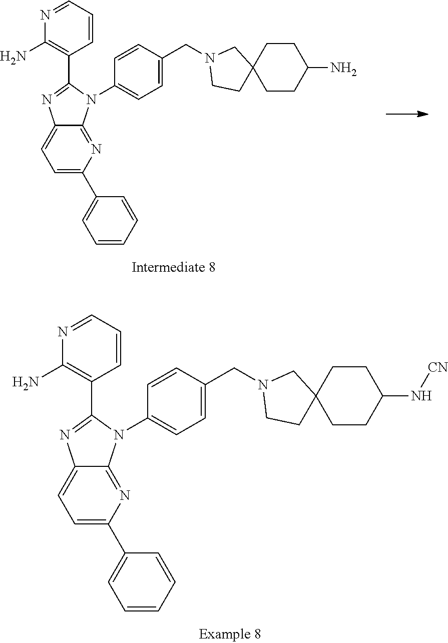 [SMILES: Nc1ncccc1-c1nc2ccc(-c3ccccc3)nc2n1-c1ccc(CN2CCC3(CCC(N)CC3)C2)cc1 ; N#CNC1CCC2(CC1)CCN(Cc1ccc(-n3c(-c4cccnc4N)nc4ccc(-c5ccccc5)nc43)cc1)C2]

Example 8 was prepared in a manner similar to Example 5. MS: m/z=555.3 [M+H]⁺. ¹H NMR (400 MHz, Methanol-d₄) δ 8.32 (d, J=8.4 Hz, 1H), 8.03 (d, J=8.0 Hz, 4H), 7.98-7.81 (m, 3H), 7.78-7.66 (m 2H), 7.49-7.37 (m, 3H), 6.95-6.82 (m, 1H), 3.87-3.55 (m, 2H), 3.50-3.38 (m, 2H), 3.26-3.01 (m, 2H), 2.27-2.11 (m, 1H), 2.11-1.74 (m, 6H), 1.68-1.40 (m, 4H).

### Example 9: (R)—N-(1-(4-(2-(2-Aminopyridin-3-yl)-5-phenyl-3H-imidazo[4,5-b]pyridin-3-yl)benzyl)piperidin-3-yl)cyanamide

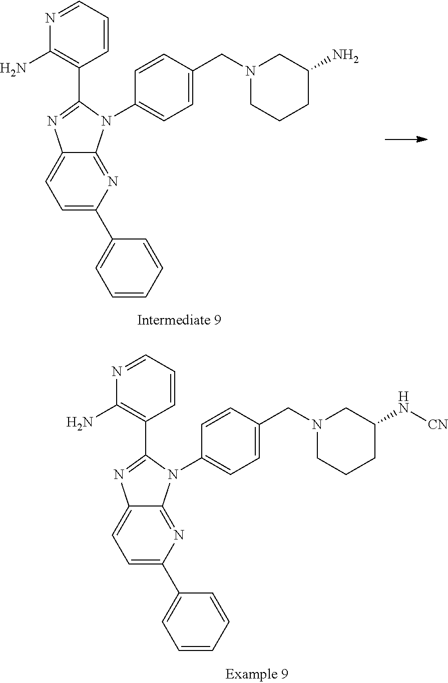 [SMILES: Nc1ncccc1-c1nc2ccc(-c3ccccc3)nc2n1-c1ccc(CN2CCC[C@@H](N)C2)cc1 ; N#CN[C@@H]1CCCN(Cc2ccc(-n3c(-c4cccnc4N)nc4ccc(-c5ccccc5)nc43)cc2)C1]

Example 9 was prepared in a manner similar to Example 5. MS: m/z=501.3 [M+H]⁺. ¹H NMR (400 MHz, Methanol-d₄) δ 8.15 (d, J=8.4 Hz, 1H), 8.03-8.00 (m, 2H), 7.97 (dd, J=4.8, 1.6 Hz, 1H), 7.90 (d, J=8.4 Hz, 1H), 7.50 (d, J=8.4 Hz, 2H), 7.43-7.35 (m, 5H), 7.26 (dd, J=7.6, 2.0 Hz, 1H), 6.47 (dd, J=7.6, 5.2 Hz 1H), 3.65-3.55 (m, 2H), 3.25-3.17 (m, 1H), 2.90-2.82 (m, 1H), 2.72-2.64 (m, 1H), 2.22-2.08 (m, 2H), 1.95-1.87 (m, 1H), 1.83-1.75 (m, 1H), 1.66-1.58 (m, 1H), 1.44-1.35 (m, 1H).

### Example 10: (R)—N-(1-(4-(2-(2-Aminopyridin-3-yl)-5-phenyl-3H-imidazo[4,5-b]pyridin-3-yl)benzyl)pyrrolidin-3-yl)cyanamide

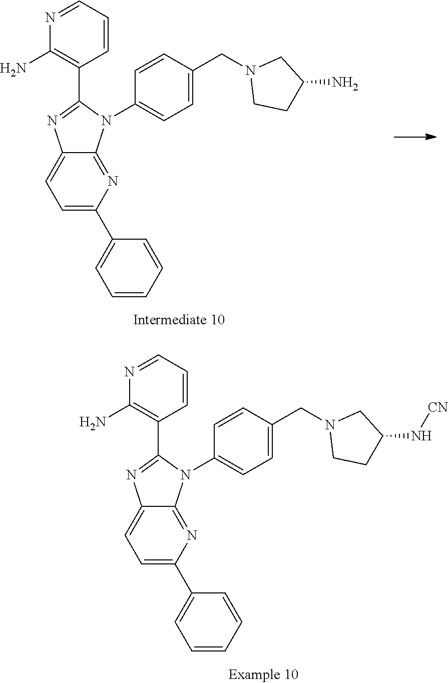 [SMILES: Nc1ncccc1-c1nc2ccc(-c3ccccc3)nc2n1-c1ccc(CN2CC[C@@H](N)C2)cc1 ; N#CN[C@@H]1CCN(Cc2ccc(-n3c(-c4cccnc4N)nc4ccc(-c5ccccc5)nc43)cc2)C1]

To a solution of Intermediate 10 (150 mg, 324 μmol) in MeOH (10 mL) were added AcONa (39.9 mg, 487 μmol) and BrCN (170 mg, 1.60 mmol). The mixture was stirred at 25° C. for 2 hr. The reaction mixture was diluted with CH₂Cl₂ (20 mL) and washed with sat. NaHCO₃ (20 mL×2), dried over Na₂SO₄, filtered, and concentrated under reduced pressure to give a residue. The residue was purified by silica gel flash chromatography (Eluent of 0~5% MeOH in CH₂Cl₂), and then purified byprep-HPLC (column: Phenomenex C18 150*25 mm 10 μm; mobile phase: [water (NH₄HCO₃)-ACN]; B %: 33%-63%, 14 min) to give (R)—N-(1-(4-(2-(2-aminopyridin-3-yl)-5-phenyl-3H-imidazo[4,5-b]pyridin-3-yl)benzyl)pyrrolidin-3-yl)cyanamide (Example 10, 13.9 mg, yield: 8.6%) as a yellow solid. MS: m/z=487.4 [M+H]⁺. ¹H NMR (400 MHz, Methanol-d₄) δ 8.18 (d, J=8.4 Hz, 1H), 8.05-8.01 (m, 2H), 7.98 (dd, J=4.8, 1.6 Hz, 1H), 7.93 (d, J=8.4 Hz, 1H), 7.55 (d, J=8.4 Hz, 2H), 7.45-7.40 (m, 4H), 7.37 (d, J=7.2 Hz, 1H), 7.30 (dd, J=7.6, 1.6 Hz, 1H), 6.48 (dd, J=8.0, 5.2 Hz, 1H), 3.83-3.75 (m, 2H), 3.71 (d, J=12.8 Hz, 1H), 2.84-2.78 (m, 2H), 2.66-2.56 (m, 2H), 2.30-2.23 (m, 1H), 1.86-1.77 (m, 1H).

### Example 11: N-(1-(4-(2-(2-Aminopyridin-3-yl)-6-phenyl-3H-imidazo[4,5-b]pyridin-3-yl)benzyl)piperidin-4-yl)cyanamide

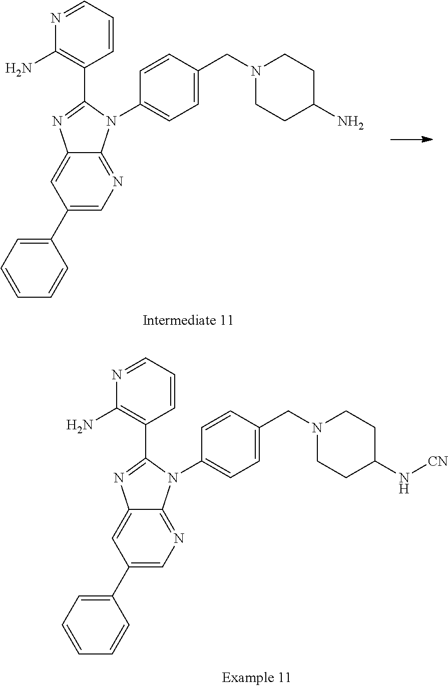 [SMILES: Nc1ncccc1-c1nc2cc(-c3ccccc3)cnc2n1-c1ccc(CN2CCC(N)CC2)cc1 ; N#CNC1CCN(Cc2ccc(-n3c(-c4cccnc4N)nc4cc(-c5ccccc5)cnc43)cc2)CC1]

Example 11 was prepared in a manner similar to Example 1. MS: m/z=501.1 [M+H]⁺. ¹H NMR (400 MHz, Dimethylsulfoxide-d₆) δ 8.63 (d, J=1.6 Hz, 1H), 8.47 (d, J=1.6 Hz, 1H), 8.00 (dd, J=4.8, 1.6 Hz, 1H), 7.79 (d, J=7.2 Hz, 2H), 7.53 (dd, J=7.6, 7.6 Hz, 2H), 7.47-7.39 (m, 4H), 7.19 (dd, J=7.6, 1.6 Hz, 1H), 7.05 (br s, 2H), 6.88 (d, J=4.0 Hz, 1H), 6.39 (dd, J=7.6, 4.8 Hz, 1H), 3.55 (s, 2H), 3.05-3.00 (m, 1H), 2.79-2.71 (m, 2H), 2.14-2.08 (m, 2H), 1.86-1.77 (m, 2H), 1.50-1.42 (m, 2H).

### Example 12: 4-(4-(2-(2-Aminopyridin-3-yl)-5-phenyl-3H-imidazo[4,5-b]pyridin-3-yl)benzyl)-1,4-diazepane-1-carbonitrile

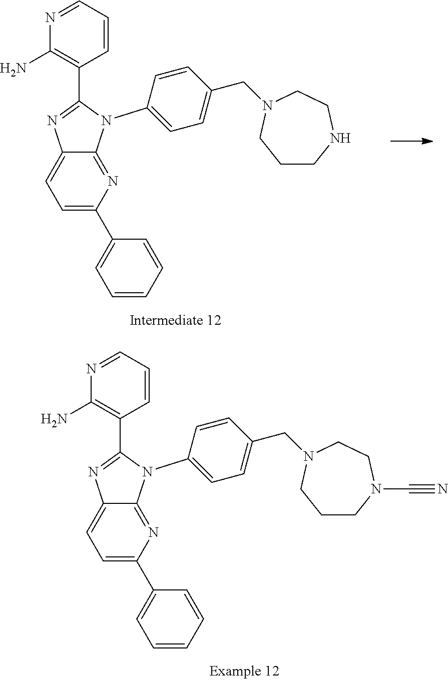 [SMILES: Nc1ncccc1-c1nc2ccc(-c3ccccc3)nc2n1-c1ccc(CN2CCCNCC2)cc1 ; N#CN1CCCN(Cc2ccc(-n3c(-c4cccnc4N)nc4ccc(-c5ccccc5)nc43)cc2)CC1]

Example 12 was prepared in a manner similar to Example 1. MS: m/z=501.2 [M+H]⁺. ¹H NMR (400 MHz, Dimethylsulfoxide-d₆) δ 8.27 (d, J=8.4 Hz, 1H), 8.04-7.97 (m, 4H), 7.52-7.39 (m, 7H), 7.15 (dd, J=7.2, 1.6 Hz, 1H), 7.03 (br s, 2H), 6.39 (dd, J=7.6, 4.8 Hz, 1H), 3.77 (s, 2H), 3.38-3.33 (m, 4H), 2.75-2.71 (m, 2H), 2.70-2.66 (m, 2H), 1.85-1.80 (m, 2H).

### Example 13: N-(7-(4-(2-(2-Aminopyridin-3-yl)-5-phenyl-3H-imidazo[4,5-b]pyridin-3-yl)benzyl)-7-azaspiro[3.5]nonan-2-yl)cyanamide

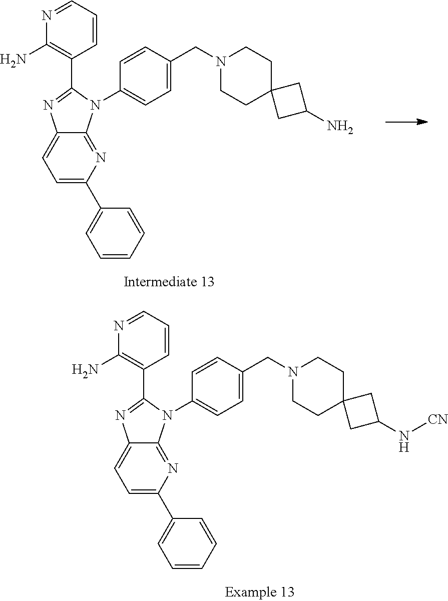 [SMILES: Nc1ncccc1-c1nc2ccc(-c3ccccc3)nc2n1-c1ccc(CN2CCC3(CC2)CC(N)C3)cc1 ; N#CNC1CC2(CCN(Cc3ccc(-n4c(-c5cccnc5N)nc5ccc(-c6ccccc6)nc54)cc3)CC2)C1]

Example 13 was prepared in a manner similar to Example 5. MS: m/z=541.3 [M+H]⁺. ¹H NMR (400 MHz, Dimethylsulfoxide-d₆) δ 8.27 (d, J=8.4 Hz, 1H), 8.06-7.95 (m, 4H), 7.49-7.39 (m, 7H), 7.14 (dd, J=8.0, 2.0 Hz, 1H), 7.03 (br s, 2H), 6.36 (dd, J=7.6, 4.8 Hz, 1H), 3.52-3.49 (m, 2H), 2.38-2.30 (m, 4H), 2.28-2.22 (m, 2H), 2.15-2.10 (m, 2H), 1.69-1.62 (m, 2H), 1.55-1.48 (m, 4H).

### Example 14: N-(1-(4-(2-(2-Aminopyridin-3-yl)-5-phenyl-3H-imidazo[4,5-b]pyridin-3-yl)benzyl)piperidin-4-yl)-N-methylcyanamide

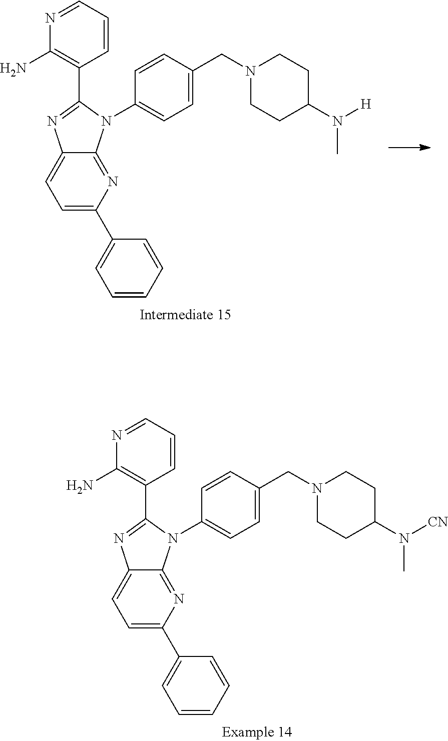 [SMILES: CNC1CCN(Cc2ccc(-n3c(-c4cccnc4N)nc4ccc(-c5ccccc5)nc43)cc2)CC1 ; CN(C#N)C1CCN(Cc2ccc(-n3c(-c4cccnc4N)nc4ccc(-c5ccccc5)nc43)cc2)CC1]

Example 14 was prepared in a manner similar to Example 5. MS: m/z=515.3 [M+H]⁺. ¹H NMR (400 MHz, Methanol-d₄) δ 8.19 (d, J=8.0 Hz, 1H), 8.03 (d, J=7.6 Hz, 2H), 7.98 (d, J=4.8 Hz, 1H), 7.94 (d, J=8.8 Hz, 1H), 7.53-7.51 (m, 2H), 7.47-7.40 (m, 4H), 7.39-7.31 (m, 2H), 6.47 (dd, J=7.6, 5.2 Hz, 1H), 3.62 (s, 2H), 3.34-3.33 (m, 1H), 2.98-2.96 (m, 2H), 2.89 (s, 3H), 2.21-2.11 (m, 2H), 2.03-1.97 (m, 2H), 1.75-1.63 (m, 2H).

### Example 15: 7-(4-(2-(2-Aminopyridin-3-yl)-5-phenyl-3H-imidazo[4,5-b]pyridin-3-yl)benzyl)-2,7-diazaspiro[3.5]nonane-2-carbonitrile

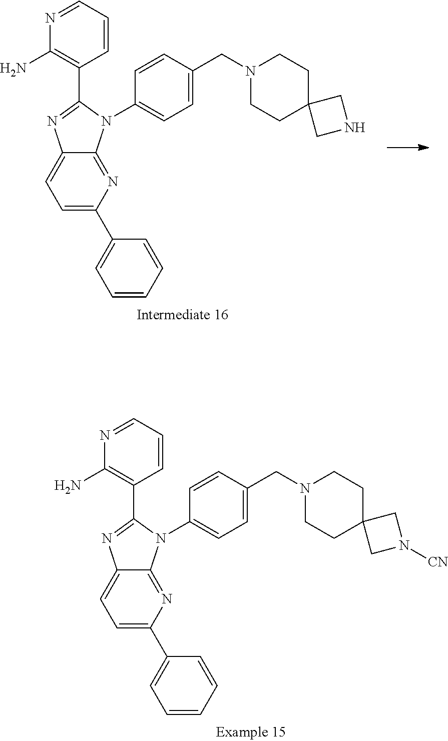 [SMILES: Nc1ncccc1-c1nc2ccc(-c3ccccc3)nc2n1-c1ccc(CN2CCC3(CC2)CNC3)cc1 ; N#CN1CC2(CCN(Cc3ccc(-n4c(-c5cccnc5N)nc5ccc(-c6ccccc6)nc54)cc3)CC2)C1]

Example 15 was prepared in a manner similar to Example 1. MS: m/z=527.3 [M+H]⁺. ¹H NMR (400 MHz, Dimethylsulfoxide-d₆) δ 8.26 (d, J=8.4 Hz, 1H), 8.04-7.97 (m, 4H), 7.48-7.39 (m, 7H), 7.18-7.13 (m, 1H), 7.02 (br s, 2H), 6.37 (dd, J=7.2, 4.8 Hz, 1H), 3.86 (s, 4H), 3.51 (s, 2H), 2.36-2.25 (m, 4H), 1.79-1.70 (m, 4H).

### Example 16: 8-(4-(2-(2-Aminopyridin-3-yl)-5-phenyl-3H-imidazo[4,5-b]pyridin-3-yl)benzyl)-2,8-diazaspiro[4.5]decane-2-carbonitrile

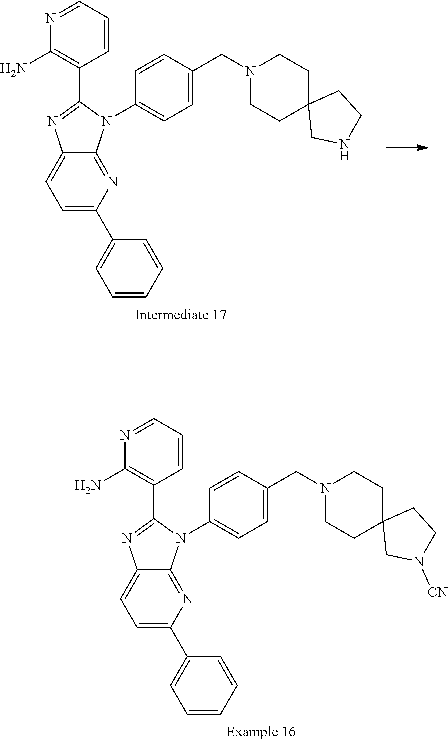 [SMILES: Nc1ncccc1-c1nc2ccc(-c3ccccc3)nc2n1-c1ccc(CN2CCC3(CCNC3)CC2)cc1 ; N#CN1CCC2(CCN(Cc3ccc(-n4c(-c5cccnc5N)nc5ccc(-c6ccccc6)nc54)cc3)CC2)C1]

Example 16 was prepared in a manner similar to Example 5. MS: m/z=541.4 [M+H]⁺. ¹H NMR (400 MHz, Methanol-d₄) δ 8.19 (d, J=8.0 Hz, 1H), 8.02 (d, J=7.6 Hz, 2H), 7.98 (d, J=4.8 Hz, 1H), 7.94 (d, J=8.4 Hz, 1H), 7.53 (d, J=8.4 Hz, 2H), 7.46-7.39 (m, 4H), 7.38-7.36 (m, 1H), 7.34-7.29 (m, 1H), 6.46 (dd, J=8.0, 5.6 Hz, 1H), 3.64 s, 2H), 3.53-3.43 (m, 4H), 2.61-2.54 (m, 2H), 2.48-2.41 (m, 2H), 1.81 (t, J=7.6 Hz, 2H), 1.65-1.62 (m, 4H).

### Example 17: 9-(4-(2-(2-Aminopyridin-3-yl)-5-phenyl-3H-imidazo[4,5-b]pyridin-3-yl)benzyl)-3,9-diazaspiro[5.5]undecane-3-carbonitrile

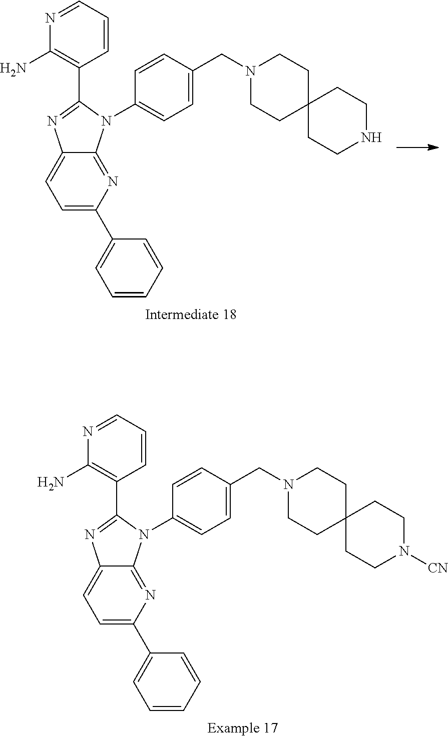 [SMILES: Nc1ncccc1-c1nc2ccc(-c3ccccc3)nc2n1-c1ccc(CN2CCC3(CCNCC3)CC2)cc1 ; N#CN1CCC2(CC1)CCN(Cc1ccc(-n3c(-c4cccnc4N)nc4ccc(-c5ccccc5)nc43)cc1)CC2]

Example 17 was prepared in a manner similar to Example 5. MS: m/z=555.4 [M+H]⁺. ¹H NMR (400 MHz, Methanol-d₄) δ 8.20 (d, J=8.4 Hz, 1H), 8.03 (d, J=7.2 Hz, 2H), 7.98 (dd, J=4.8, 1.6 Hz, 1H), 7.94 (d, J=8.8 Hz, 1H), 7.53 (d, J=8.4 Hz, 2H), 7.47-7.30 (m, 6H), 6.47 (dd, J=7.6, 5.2 Hz, 1H), 3.64 (s, 2H), 3.26-3.21 (m, 4H), 2.56-2.46 (m, 4H), 1.64-1.56 (m, 8H).

### Example 18: 6-(4-(2-(2-Aminopyridin-3-yl)-5-phenyl-3H-imidazo[4,5-b]pyridin-3-yl)benzyl)-2,6-diazaspiro[3.4]octane-2-carbonitrile

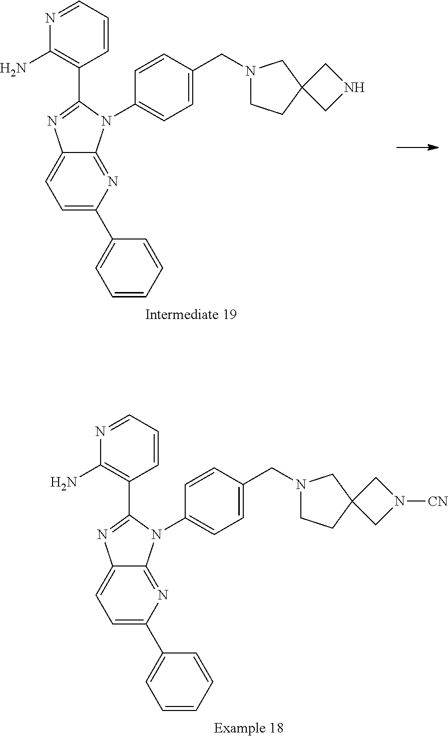 [SMILES: Nc1ncccc1-c1nc2ccc(-c3ccccc3)nc2n1-c1ccc(CN2CCC3(CNC3)C2)cc1 ; N#CN1CC2(CCN(Cc3ccc(-n4c(-c5cccnc5N)nc5ccc(-c6ccccc6)nc54)cc3)C2)C1]

Example 18 was prepared in a manner similar to Example 5. MS: m/z=513.3 [M+H]⁺. ¹H NMR (400 MHz, Methanol-d₄) δ 8.19 (d, J=8.4 Hz, 1H), 8.03 (d, J=7.2 Hz, 2H), 7.99 (d, J=3.6 Hz, 1H), 7.94 (d, J=8.4 Hz, 1H), 7.52 (d, J=8.4 Hz, 2H), 7.46-7.31 (m, 6H), 6.49 (dd, J=7.6, 4.8 Hz, 1H), 4.11 (s, 4H), 3.71 (s, 2H), 2.84 (s, 2H), 2.65 (t, J=7.2 Hz, 2H), 2.14 (t, J=7.2 Hz, 2H).

### Example 19: 7-(4-(2-(2-Aminopyridin-3-yl)-5-phenyl-3H-imidazo[4,5-b]pyridin-3-yl)benzyl)-2,7-diazaspiro[4.4]nonane-2-carbonitrile

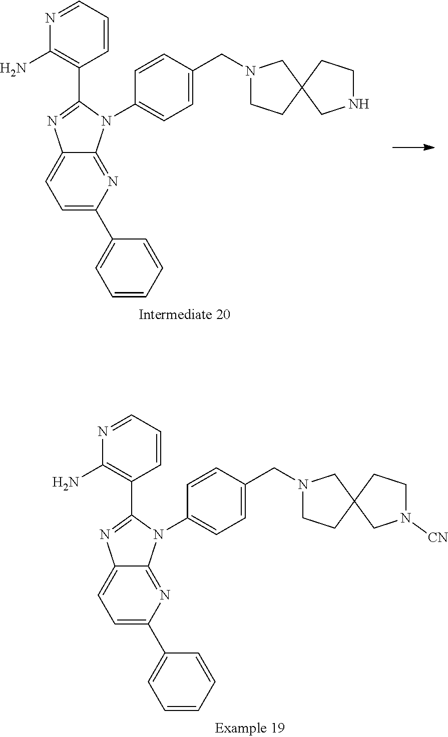 [SMILES: Nc1ncccc1-c1nc2ccc(-c3ccccc3)nc2n1-c1ccc(CN2CCC3(CCNC3)C2)cc1 ; N#CN1CCC2(CCN(Cc3ccc(-n4c(-c5cccnc5N)nc5ccc(-c6ccccc6)nc54)cc3)C2)C1]

Example 19 was prepared in a manner similar to Example 1. MS: m/z=527.3 [M+H]⁺. ¹H NMR (400 MHz, Dimethylsulfoxide-d₆) δ 8.25 (d, J=8.4 Hz, 1H), 8.07-7.88 (m, 4H), 7.55-7.34 (m, 7H), 7.14 (dd, J=7.6, 2.0 Hz, 1H), 7.00 (br s, 2H), 6.37 (dd, J=7.6, 4.8 Hz, 1H), 3.66 (s, 2H), 3.40-3.35 (m, 2H), 3.27-3.25 (m, 1H), 3.24-3.20 (m, 1H), 2.64-2.60 (m, 1H), 2.56-2.51 (m, 2H), 2.39-2.37 (m, 1H), 1.89-1.72 (m, 4H).

### Example 20: 2-(4-(2-(2-Aminopyridin-3-yl)-5-phenyl-3H-imidazo[4,5-b]pyridin-3-yl)benzyl)-2,8-diazaspiro[4.5]decane-8-carbonitrile

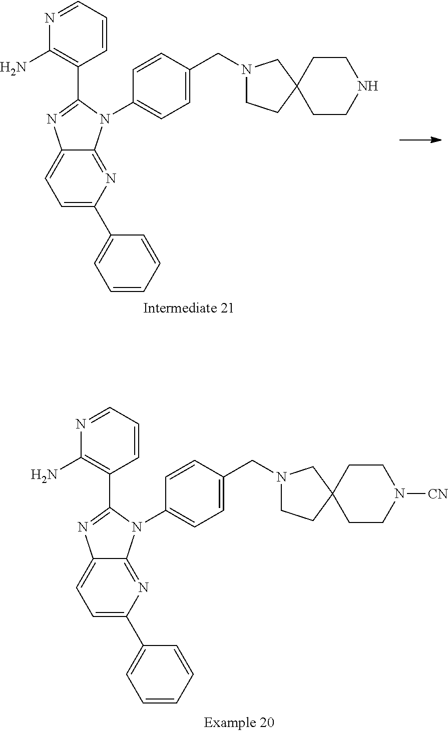 [SMILES: Nc1ncccc1-c1nc2ccc(-c3ccccc3)nc2n1-c1ccc(CN2CCC3(CCNCC3)C2)cc1 ; N#CN1CCC2(CC1)CCN(Cc1ccc(-n3c(-c4cccnc4N)nc4ccc(-c5ccccc5)nc43)cc1)C2]

Example 20 was prepared in a manner similar to Example 1. MS: m/z=541.4 [M+H]⁺. ¹H NMR (400 MHz, Methanol-d₄) δ 8.19 (d, J=8.4 Hz, 1H), 8.06-8.01 (m, 2H), 7.99-7.96 (m, 1H), 7.94 (d, J=8.4 Hz, 1H), 7.54 (d, J=8.4 Hz, 2H), 7.52-7.33 (m, 6H), 6.48 (dd, J=7.6, 4.8 Hz, 1H), 3.71 (s, 2H), 3.63-3.43 (m, 2H), 3.28-3.18 (m, 4H), 2.72-2.66 (m, 2H), 2.50 (s, 2H), 1.75-1.65 (m, 4H).

### Example 21: 2-(4-(2-(2-Aminopyridin-3-yl)-5-phenyl-3H-imidazo[4,5-b]pyridin-3-yl)benzyl)-2,7-diazaspiro[4.5]decane-7-carbonitrile

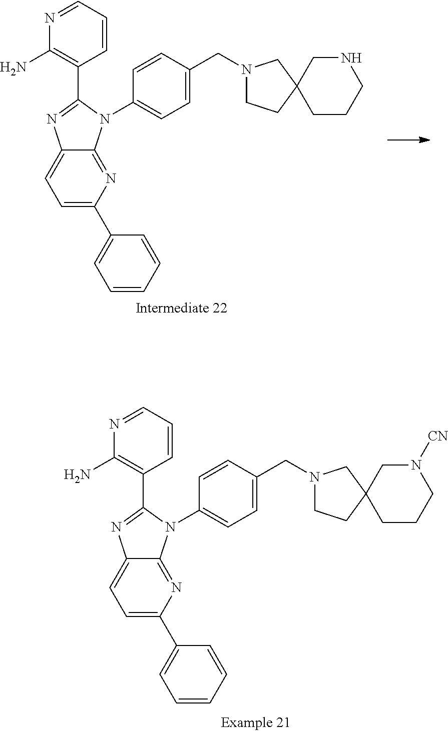 [SMILES: Nc1ncccc1-c1nc2ccc(-c3ccccc3)nc2n1-c1ccc(CN2CCC3(CCCNC3)C2)cc1 ; N#CN1CCCC2(CCN(Cc3ccc(-n4c(-c5cccnc5N)nc5ccc(-c6ccccc6)nc54)cc3)C2)C1]

Example 21 was prepared in a manner similar to Example 1. MS: m/z=541.4 [M+H]⁺. ¹H NMR (400 MHz, Methanol-d₄) δ 8.19 (d, J=8.4 Hz, 1H), 8.08-8.01 (m, 2H), 7.98 (dd, J=4.8, 1.6 Hz, 1H), 7.94 (d, J=8.4 Hz, 1H), 7.54 (d, J=8.4 Hz, 2H), 7.47-7.32 (m, 6H), 6.49 (dd, J=7.6, 5.2 Hz, 1H), 3.72 (s, 2H), 3.23-3.11 (m, 3H), 3.08-3.00 (m, 1H), 2.85-2.80 (m, 1H), 2.75 (d, J=9.6 Hz, 1H), 2.63-2.54 (m, 1H), 2.31 (d, J=9.6 Hz, 1H), 1.79-1.72 (m, 1H), 1.70-1.51 (m, 5H).

### Example 22: 2-(4-(2-(2-Aminopyridin-3-yl)-5-phenyl-3H-imidazo[4,5-b]pyridin-3-yl)benzyl)-2,7-diazaspiro[3.5]nonane-7-carbonitrile

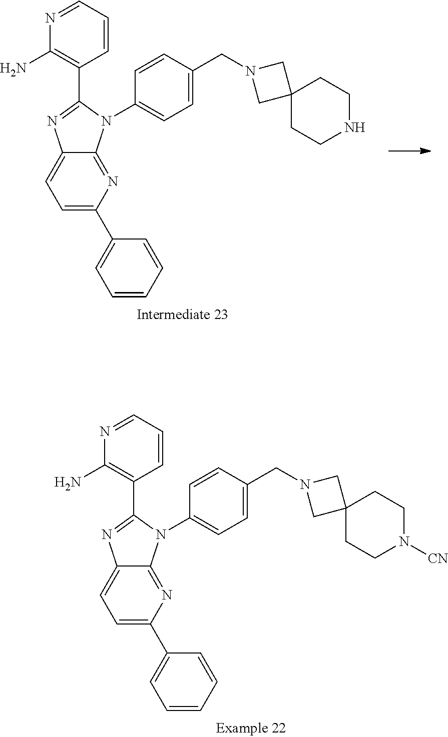 [SMILES: Nc1ncccc1-c1nc2ccc(-c3ccccc3)nc2n1-c1ccc(CN2CC3(CCNCC3)C2)cc1 ; N#CN1CCC2(CC1)CN(Cc1ccc(-n3c(-c4cccnc4N)nc4ccc(-c5ccccc5)nc43)cc1)C2]

Example 22 was prepared in a manner similar to Example 5. MS: m/z=527.3 [M+H]⁺. ¹H NMR (400 MHz, Methanol-d₄) δ 8.19 (d, J=8.8 Hz, 1H), 8.04-8.02 (m, 2H), 7.98 (dd, J=5.2, 2.0 Hz, 1H), 7.94 (d, J=8.8 Hz, 1H), 7.52-7.45 (m, 4H), 7.45-7.39 (m, 3H), 7.33 (dd, J=7.6, 2.0 Hz, 1H), 6.48 (dd, J=7.6, 5.2 Hz, 1H), 3.78 (s, 2H), 3.22-3.18 (m, 8H), 1.89-1.85 (m, 4H).

### Example 23: (R)-7-(4-(2-(2-Aminopyridin-3-yl)-5-phenyl-3H-imidazo[4,5-b]pyridin-3-yl)benzyl)-2,7-diazaspiro[4.4]nonane-2-carbonitrile

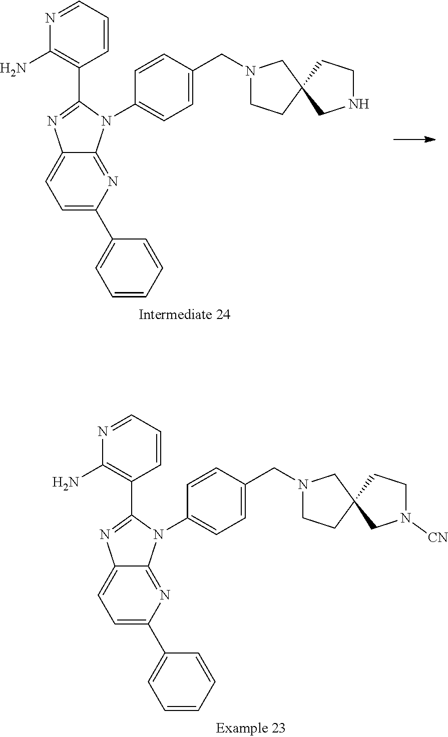 [SMILES: Nc1ncccc1-c1nc2ccc(-c3ccccc3)nc2n1-c1ccc(CN2CC[C@@]3(CCNC3)C2)cc1 ; N#CN1CC[C@@]2(CCN(Cc3ccc(-n4c(-c5cccnc5N)nc5ccc(-c6ccccc6)nc54)cc3)C2)C1]

Example 23 was prepared in a manner similar to Example 1. MS: m/z=527.2 [M+H]⁺. ¹H NMR (400 MHz, Methanol-d₄) δ 8.27 (d, J=8.4 Hz, 1H), 8.05-7.97 (m, 4H), 7.51-7.39 (m, 7H), 7.16 (dd, J=7.6, 2.0 Hz, 1H), 7.02 (br s, 2H), 6.39 (dd, J=7.6, 4.8 Hz, 1H), 3.68 (s, 2H), 3.41-3.39 (m, 2H), 3.29-3.21 (m, 2H), 2.67-2.64 (m, 2H), 2.47-2.30 (m, 2H), 1.92-1.74 (m, 4H).

### Example 24: 9-(4-(2-(2-Aminopyridin-3-yl)-5-phenyl-3H-imidazo[4,5-b]pyridin-3-yl)benzyl)-2,9-diazaspiro[5.5]undecane-2-carbonitrile

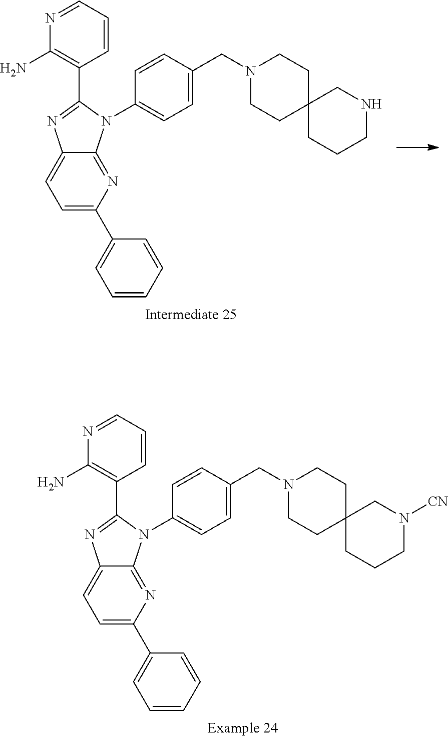 [SMILES: Nc1ncccc1-c1nc2ccc(-c3ccccc3)nc2n1-c1ccc(CN2CCC3(CCCNC3)CC2)cc1 ; N#CN1CCCC2(CCN(Cc3ccc(-n4c(-c5cccnc5N)nc5ccc(-c6ccccc6)nc54)cc3)CC2)C1]

Example 24 was prepared in a manner similar to Example 1. MS: m/z=555.4 [M+H]⁺. ¹H NMR (400 MHz, Methanol-d₄) δ 8.10 (d, J=8.4 Hz, 1H), 7.96 (dd, J=8.4, 1.6 Hz, 2H), 7.92 (dd, J=4.8, 1.2 Hz, 1H), 7.85 (d, J=8.4 Hz, 1H), 7.47-7.42 (m, 2H), 7.38-7.31 (m, 5H), 7.20 (dd, J=7.6, 2.0 Hz, 1H), 6.38 (dd, J=7.6, 4.8 Hz, 1H), 3.61 (s, 2H), 3.28-3.26 (m, 2H), 3.16-3.12 (m, 2H), 2.57-2.48 (m, 2H), 2.47-2.38 (m, 2H), 1.63-1.49 (m, 6H), 1.44-1.39 (m, 2H).

### Example 25: (2S,6S)-4-(4-(2-(2-Aminopyridin-3-yl)-5-phenyl-3H-imidazo[4,5-b]pyridin-3-yl)benzyl)-2,6-dimethylpiperazine-1-carbonitrile

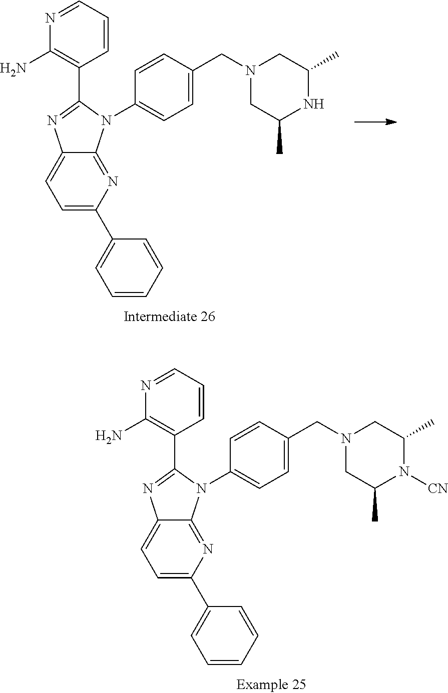 [SMILES: C[C@H]1CN(Cc2ccc(-n3c(-c4cccnc4N)nc4ccc(-c5ccccc5)nc43)cc2)C[C@H](C)N1 ; C[C@H]1CN(Cc2ccc(-n3c(-c4cccnc4N)nc4ccc(-c5ccccc5)nc43)cc2)C[C@H](C)N1C#N]

To a solution of Intermediate 26 (130 mg, 266 μmol) in acetone (2 mL) were added K₂CO₃ (110 mg, 797 μmol) and BrCN (141 mg, 1.33 mmol). The mixture was stirred at 25° C. for 1 hr. The reaction was concentrated under reduced pressure. After purification by prep-HPLC (column: Phenomenex C18 150*25 mm 10 μm; mobile phase: [water (NH₄HCO₃)-ACN]; B %: 45%-75%, 14 min), (2S,6S)-4-(4-(2-(2-aminopyridin-3-yl)-5-phenyl-3H-imidazo[4,5-b]pyridin-3-yl)benzyl)-2,6-dimethylpiperazine-1-carbonitrile (Example 25, 17.8 mg, yield: 13%) was obtained as a yellow lyophilized powder. MS: m/z=515.3 [M+H]⁺. ¹H NMR (400 MHz, Dimethylsulfoxide-d₆) δ 8.27 (d, J=8.4 Hz, 1H), 8.04-7.98 (m, 4H), 7.50-7.44 (m, 6H), 7.41-7.37 (m, 1H), 7.15 (dd, J=7.6, 1.6 Hz, 1H), 7.04 (br s, 2H), 6.38 (dd, J=7.6, 4.8 Hz, 1H), 3.65 (d, J=13.6 Hz, 1H), 3.58-3.54 (m, 2H), 3.52 (d, J=13.6 Hz, 1H), 2.57-2.55 (m, 2H), 2.21 (dd, J=11.2, 6.0 Hz, 2H), 1.24 (d, J=6.8 Hz, 6H).

### Example 26: (2R,6R)-4-(4-(2-(2-Aminopyridin-3-yl)-5-phenyl-3H-imidazo[4,5-b]pyridin-3-yl)benzyl)-2,6-dimethylpiperazine-1-carbonitrile

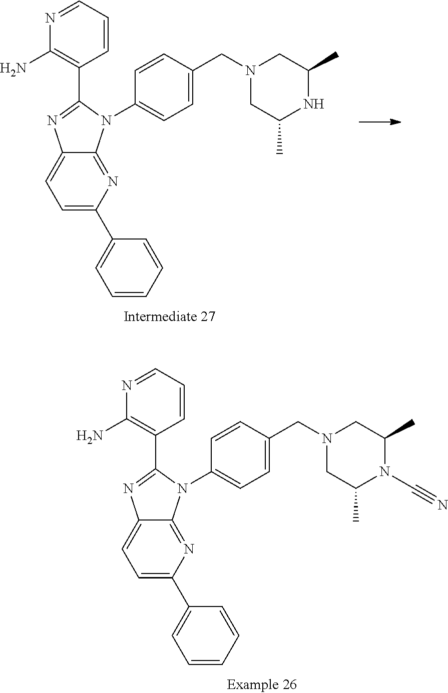 [SMILES: C[C@@H]1CN(Cc2ccc(-n3c(-c4cccnc4N)nc4ccc(-c5ccccc5)nc43)cc2)C[C@@H](C)N1 ; C[C@@H]1CN(Cc2ccc(-n3c(-c4cccnc4N)nc4ccc(-c5ccccc5)nc43)cc2)C[C@@H](C)N1C#N]

Example 26 was prepared in a manner similar to Example 25. MS: m/z=515.3 [M+H]⁺. ¹H NMR (400 MHz, Dimethylsulfoxide-d₆) δ 8.27 (d, J=8.4 Hz, 1H), 8.06-7.95 (m, 4H), 7.53-7.37 (m, 7H), 7.15 (dd, J=7.6, 1.6 Hz, 1H), 7.04 (br s, 2H), 6.38 (dd, J=7.6, 4.8 Hz, 1H), 3.65 (d, J=13.6 Hz, 1H), 3.59-3.49 (m, 3H), 2.57-2.53 (m, 2H), 2.25-2.17 (m, 2H), 1.24 (d, J=6.4 Hz, 6H).

### Example 27: (R)-4-(4-(2-(2-Aminopyridin-3-yl)-5-phenyl-3H-imidazo[4,5-b]pyridin-3-yl)benzyl)-2-methylpiperazine-1-carbonitrile

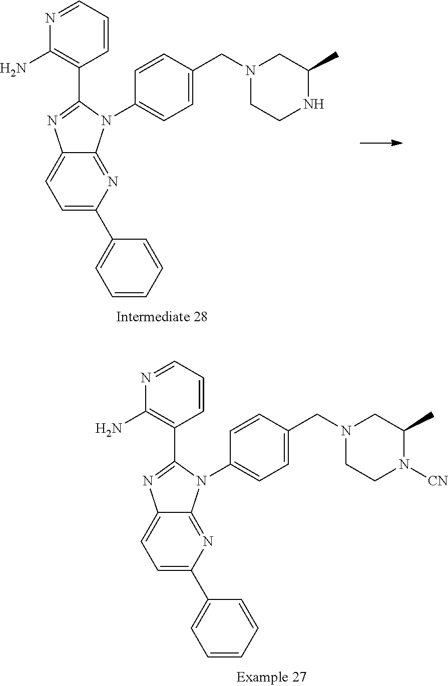 [SMILES: C[C@@H]1CN(Cc2ccc(-n3c(-c4cccnc4N)nc4ccc(-c5ccccc5)nc43)cc2)CCN1 ; C[C@@H]1CN(Cc2ccc(-n3c(-c4cccnc4N)nc4ccc(-c5ccccc5)nc43)cc2)CCN1C#N]

Example 27 was prepared in a manner similar to Example 1. MS: m/z=501.2 [M+H]⁺. ¹H NMR (400 MHz, Dimethylsulfoxide-d₆) δ 12.05 (br s, 1H), 8.55 (br s, 2H), 8.37 (d, J=8.4 Hz, 1H), 8.15 (dd, J=6.0, 1.6 Hz, 1H), 8.09-8.05 (m, 3H), 7.89-7.82 (m, 3H), 7.68 (d, J=8.4 Hz, 2H), 7.51-7.42 (m, 3H), 6.89 (dd, J=7.2, 6.0 Hz, 1H), 4.45 (s, 2H), 3.75-3.71 (m, 2H), 3.41-3.33 (m, 2H), 3.24-3.11 (m, 2H), 2.97-2.88 (m, 1H), 1.26 (J=6.0 Hz, 3H).

### Example 28: 3-(4-(2-(2-Aminopyridin-3-yl)-5-phenyl-3H-imidazo[4,5-b]pyridin-3-yl)benzyl)-3,8-diazabicyclo[3.2.1]octane-8-carbonitrile

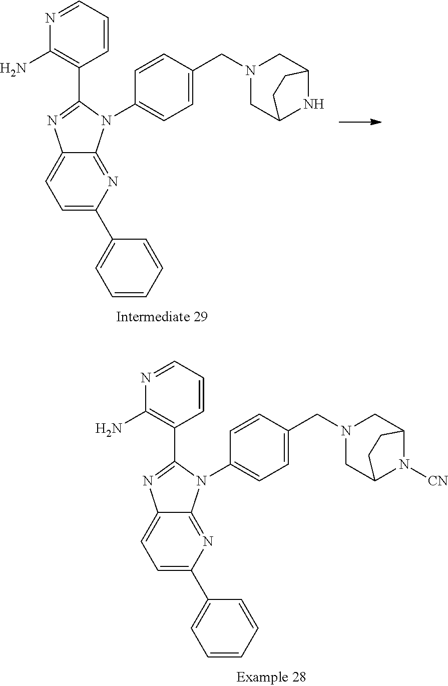 [SMILES: Nc1ncccc1-c1nc2ccc(-c3ccccc3)nc2n1-c1ccc(CN2CC3CCC(C2)N3)cc1 ; N#CN1C2CCC1CN(Cc1ccc(-n3c(-c4cccnc4N)nc4ccc(-c5ccccc5)nc43)cc1)C2]

Example 28 was prepared in a manner similar to Example 1. MS: m/z=513.3 [M+H]⁺. ¹H NMR (400 MHz, Dimethylsulfoxide-d₆) δ 8.26 (d, J=8.4 Hz, 1H), 8.06-7.96 (m, 4H), 7.54-7.44 (m, 6H), 7.43-7.37 (m, 1H), 7.13 (dd, J=7.6, 1.6 Hz, 1H), 7.04 (br s, 2H), 6.39 (dd, J=7.6, 4.8 Hz, 1H), 3.95-3.89 (m, 2H), 3.64 (s, 2H), 2.70-2.62 (m, 2H), 2.46-2.38 (m, 2H), 1.94-1.86 (m, 4H).

### Example 29: 8-(4-(2-(2-Aminopyridin-3-yl)-5-phenyl-3H-imidazo[4,5-b]pyridin-3-yl)benzyl)-3,8-diazabicyclo[3.2.1]octane-3-carbonitrile

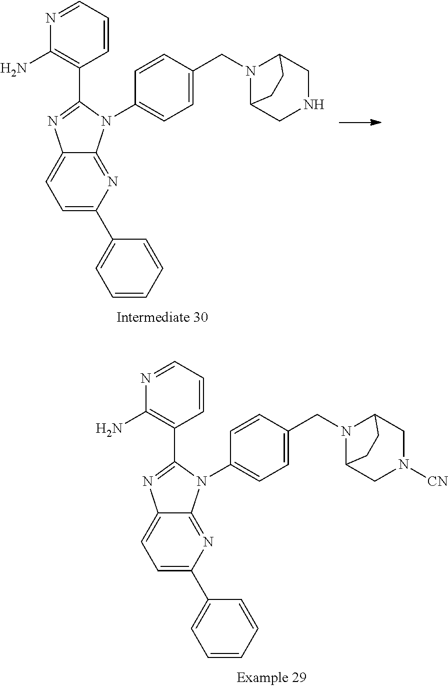 [SMILES: Nc1ncccc1-c1nc2ccc(-c3ccccc3)nc2n1-c1ccc(CN2C3CCC2CNC3)cc1 ; N#CN1CC2CCC(C1)N2Cc1ccc(-n2c(-c3cccnc3N)nc3ccc(-c4ccccc4)nc32)cc1]

Example 29 was prepared in a manner similar to Example 1. MS: m/z=513.3 [M+H]⁺. ¹H NMR (400 MHz, Dimethylsulfoxide-d₆) δ 8.26 (d, J=8.4 Hz, 1H), 8.04-7.97 (m, 4H), 7.54 (d, J=8.4 Hz, 2H), 7.49-7.40 (m, 5H), 7.14 (dd, J=7.6, 1.6 Hz, 1H), 7.00 (br s, 2H), 6.39 (dd, J=8.0, 4.8 Hz, 1H), 3.59 (s, 2H), 3.30-3.26 (m, 2H), 3.20-3.16 (m, 2H), 3.11-3.03 (m, 2H), 2.10-2.02 (m, 1H), 1.85-1.75 (m, 2H).

### Example 30: N-(1-(4-(2-(2-Aminopyridin-3-yl)-5-phenyl-3H-imidazo[4,5-b]pyridin-3-yl)benzyl)azepan-4-yl)cyanamide

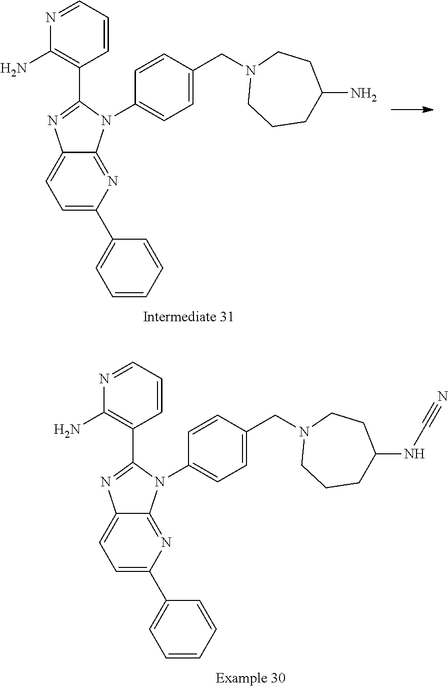 [SMILES: Nc1ncccc1-c1nc2ccc(-c3ccccc3)nc2n1-c1ccc(CN2CCCC(N)CC2)cc1 ; N#CNC1CCCN(Cc2ccc(-n3c(-c4cccnc4N)nc4ccc(-c5ccccc5)nc43)cc2)CC1]

Example 30 was prepared in a manner similar to Example 1. MS: m/z=515.2 [M+H]⁺. ¹H NMR (400 MHz, Methanol-d₄) δ 8.18 (d, J=8.4 Hz, 1H), 8.05-8.01 (m, 2H), 7.98 (dd, J=5.2, 2.0 Hz, 1H), 7.93 (d, J=8.4 Hz, 1H), 7.53 (d, J=8.4 Hz, 2H), 7.44-7.34 (m, 5H), 7.29 (dd, J=7.6, 2.0 Hz, 1H), 6.46 (dd, J=7.6, 5.2 Hz, 1H), 3.72 (s, 2H), 3.42-3.33 (m, 1H), 2.82-2.59 (m, 4H), 2.06-1.96 (m, 2H), 1.85-1.62 (m, 4H).

### Example 31: N-(1-(4-(2-(2-Aminopyridin-3-yl)-5-phenyl-3H-imidazo[4,5-b]pyridin-3-yl)benzyl)azepan-3-yl)cyanamide

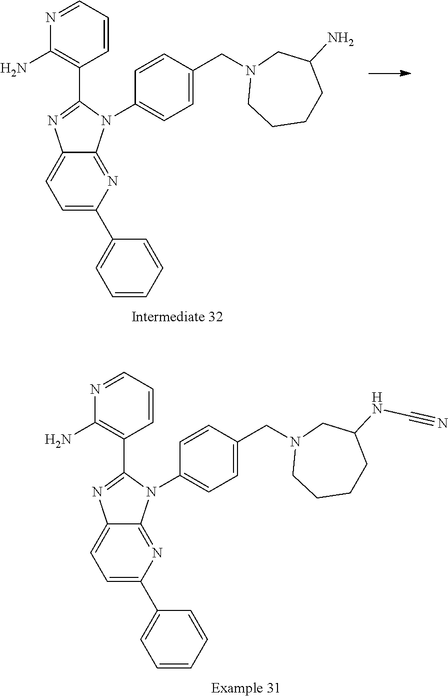 [SMILES: Nc1ncccc1-c1nc2ccc(-c3ccccc3)nc2n1-c1ccc(CN2CCCCC(N)C2)cc1 ; N#CNC1CCCCN(Cc2ccc(-n3c(-c4cccnc4N)nc4ccc(-c5ccccc5)nc43)cc2)C1]

Example 31 was prepared in a manner similar to Example 1. MS: m/z=515.4 [M+H]⁺. ¹H NMR (400 MHz, Dimethylsulfoxide-d₆) δ 8.30-8.24 (m, 1H), 8.08-7.97 (m, 4H), 7.65-7.51 (m, 2H), 7.50-7.43 (m, 4H), 7.42-7.37 (m, 1H), 7.23-7.14 (m, 1H), 7.06 (br s, 1H), 6.90-6.65 (m, 1H), 6.70-6.64 (m, 1H), 6.45-6.36 (m, 1H), 4.87 (s, 1H), 3.84-3.69 (m, 2H), 3.42-3.38 (m, 2H), 2.90-2.81 (m, 1H), 2.64-2.54 (m, 4H), 1.73-1.47 (m, 4H).

### Example 32: (S)-4-(4-(2-(2-Aminopyridin-3-yl)-5-phenyl-3H-imidazo[4,5-b]pyridin-3-yl)benzyl)-2-methylpiperazine-1-carbonitrile

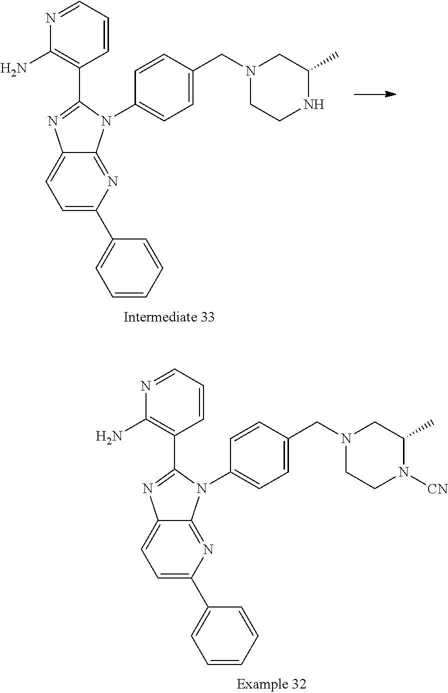 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1C#N]

To a solution of Intermediate 33 (50 mg, 105 μmol) in CH₂Cl₂ (1 mL) were added BrCN (22.3 mg, 210 μmol) and DIEA (67.9 mg, 526 μmol) at 0° C. The mixture was stirred at 0° C. for 1 hr. The mixture was concentrated to give a residue. The residue was purified by prep-TLC (100% Ethyl acetate), (S)-4-(4-(2-(2-aminopyridin-3-yl)-5-phenyl-3H-imidazo[4,5-b]pyridin-3-yl)benzyl)-2-methylpiperazine-1-carbonitrile (Example 32, 29.8 mg, 55% yield) was obtained a yellow solid. MS: m/z=501.2 [M+H]⁺. ¹H NMR (400 MHz, Dimethylsulfoxide-d₆) δ 8.27 (d, J=8.0 Hz, 1H), 8.09-7.89 (m, 4H), 7.63-7.33 (m, 7H), 7.15 (d, J=6.8 Hz, 1H), 7.01 (br s, 2H), 6.47-6.28 (m, 1H), 4.11-4.08 (m, 1H), 3.62 (s, 2H), 3.17-3.16 (m, 2H), 2.77-2.70 (m, 2H), 2.29-2.14 (m, 1H), 2.01-1.82 (m, 1H), 1.18 (d, J=6.0 Hz, 3H).

### Example 33: 7-(4-(2-(2-Aminopyridin-3-yl)-5-phenyl-3H-imidazo[4,5-b]pyridin-3-yl)benzyl)-4,7-diazaspiro[2.5]octane-4-carbonitrile

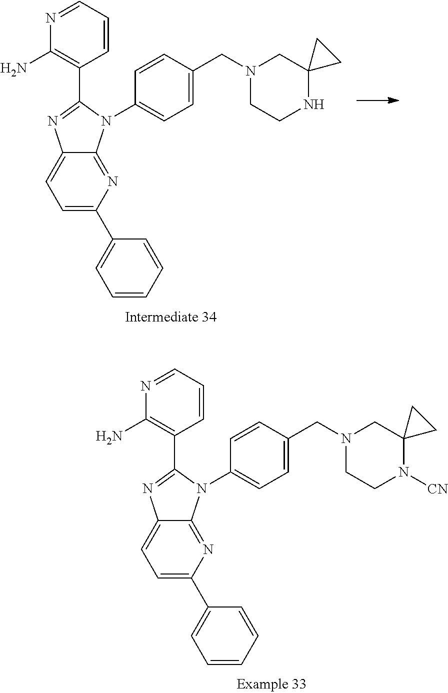 [SMILES: Nc1ncccc1-c1nc2ccc(-c3ccccc3)nc2n1-c1ccc(CN2CCNC3(CC3)C2)cc1 ; N#CN1CCN(Cc2ccc(-n3c(-c4cccnc4N)nc4ccc(-c5ccccc5)nc43)cc2)CC12CC2]

Example 33 was prepared in a manner similar to Example 32. MS: m/z=513.3 [M+H]⁺. ¹H NMR (400 MHz, Dimethylsulfoxide-d₆) δ 8.26 (d, J=8.4 Hz, 1H), 8.14-7.87 (m, 4H), 7.58-7.33 (m, 7H), 7.14 (d, J=7.2 Hz, 1H), 7.03 (br s, 2H), 6.37 (dd, J=7.2, 4.8 Hz, 1H), 3.65 (s, 2H), 2.61-2.57 (m, 2H), 2.56-2.52 (m, 2H), 2.40 (s, 2H), 0.91-0.82 (m, 2H), 0.74-0.70 (m, 2H).

### Example 34: (1S,4S)-5-(4-(2-(2-Aminopyridin-3-yl)-5-phenyl-3H-imidazo[4,5-b]pyridin-3-yl)benzyl)-2,5-diazabicyclo[2.2.1]heptane-2-carbonitrile

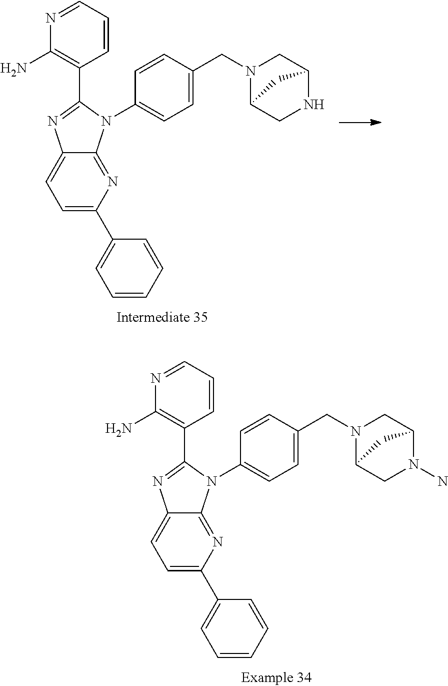 [SMILES: Nc1ncccc1-c1nc2ccc(-c3ccccc3)nc2n1-c1ccc(CN2C[C@@H]3C[C@H]2CN3)cc1 ; Nc1ncccc1-c1nc2ccc(-c3ccccc3)nc2n1-c1ccc(CN2C[C@@H]3C[C@H]2CN3N)cc1]

Example 34 was prepared in a manner similar to Example 1. MS: m/z=499.3 [M+H]⁺. ¹H NMR (400 MHz, Methanol-d₄) 8.19 (d, J=8.4 Hz, 1H), 8.05-8.02 (m, 2H), 7.98 (dd, J=5.2, 2.0 Hz, 1H), 7.94 (d, J=8.4 Hz, 1H), 7.58 (d, J=8.4 Hz, 2H), 7.47-7.36 (m, 5H), 7.33 (dd, J=7.6, 2.0 Hz, 1H), 6.49 (dd, J=7.6, 5.2 Hz, 1H), 4.16-4.09 (m, 1H), 3.95-3.85 (m, 2H), 3.67-3.59 (m, 2H), 3.43-3.37 (m, 1H), 2.94 (s, 2H), 2.07-1.99 (m, 1H), 1.88-1.79 (m, 1H).

### Example 35: (1R,4R)-5-(4-(2-(2-Aminopyridin-3-yl)-5-phenyl-3H-imidazo[4,5-b]pyridin-3-yl)benzyl)-2,5-diazabicyclo[2.2.1]heptane-2-carbonitrile

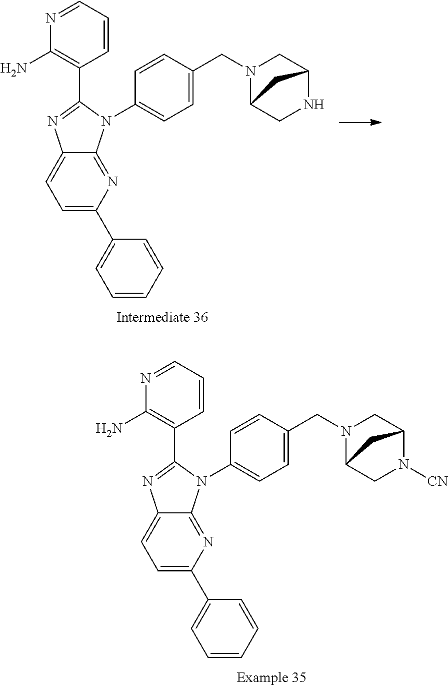 [SMILES: Nc1ncccc1-c1nc2ccc(-c3ccccc3)nc2n1-c1ccc(CN2C[C@H]3C[C@@H]2CN3)cc1 ; N#CN1C[C@H]2C[C@@H]1CN2Cc1ccc(-n2c(-c3cccnc3N)nc3ccc(-c4ccccc4)nc32)cc1]

Example 35 was prepared in a manner similar to Example 1. MS: m/z=499.3 [M+H]⁺. ¹H NMR (400 MHz, Dimethylsulfoxide-d₆) δ 8.26 (d, J=8.4 Hz, 1H), 8.03-7.97 (m, 4H), 7.56-7.37 (m, 7H), 7.16 (dd, J=7.6, 2.0 Hz, 1H), 6.99 (br s, 2H), 6.40 (dd, J=7.6, 5.2 Hz, 1H), 4.17-4.13 (m, 1H), 3.88-3.82 (m, 2H), 3.53-3.50 (m, 3H), 2.85 (dd, J=10.4, 2.0 Hz, 1H), 2.73 (d, J=10.4 Hz, 1H), 1.9 (d, J=10.4 Hz, 1H), 1.71 (d, J=10.4 Hz, 1H).

### Example 36: (2S,6R)-4-(4-(2-(2-Aminopyridin-3-yl)-5-phenyl-3H-imidazo[4,5-b]pyridin-3-yl)benzyl)-2,6-dimethylpiperazine-1-carbonitrile

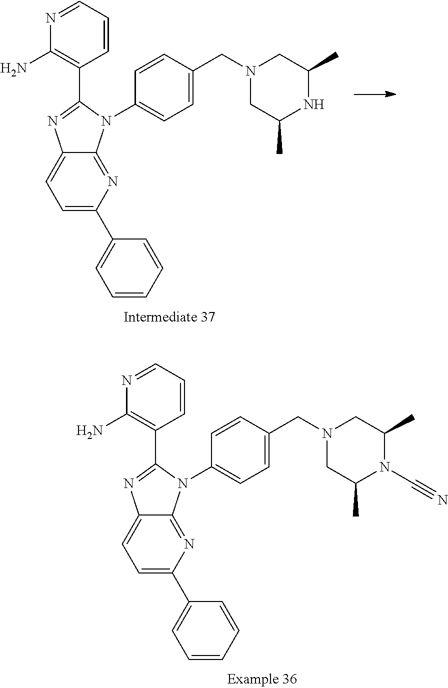 [SMILES: C[C@@H]1CN(Cc2ccc(-n3c(-c4cccnc4N)nc4ccc(-c5ccccc5)nc43)cc2)C[C@H](C)N1 ; C[C@@H]1CN(Cc2ccc(-n3c(-c4cccnc4N)nc4ccc(-c5ccccc5)nc43)cc2)C[C@H](C)N1C#N]

Example 36 was prepared in a manner similar to Example 25. MS: m/z=515.4 [M+H]⁺. ¹H NMR (400 MHz, Methanol-d₄) δ 8.32 (d, J=8.4 Hz, 1H), 8.06-8.00 (m, 4H), 7.93-7.84 (m, 3H), 7.74 (d, J=7.6 Hz, 2H), 7.47-7.40 (m, 3H), 6.87 (t, J=6.4 Hz, 1H), 4.55 (s, 2H), 3.75-3.70 (m, 2H), 3.59-3.56 (m, 2H), 3.08-3.01 (m, 2H), 1.42 (d, J=6.4 Hz, 6H).

### Example 37: 5-(4-(2-(2-Aminopyridin-3-yl)-5-phenyl-3H-imidazo[4,5-b]pyridin-3-yl)benzyl)-2,5-diazabicyclo[2.2.2]octane-2-carbonitrile

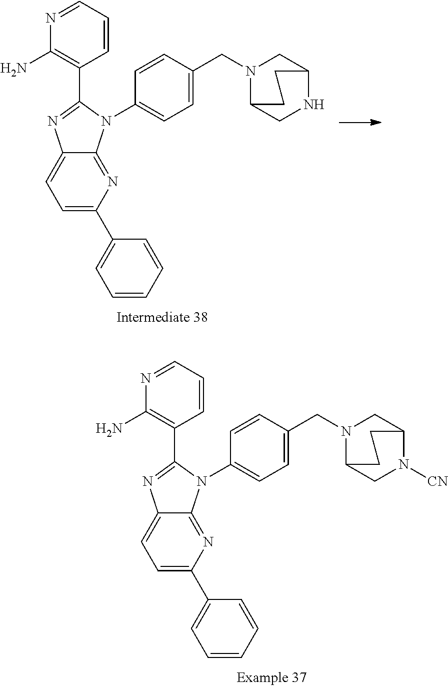 [SMILES: Nc1ncccc1-c1nc2ccc(-c3ccccc3)nc2n1-c1ccc(CN2CC3CCC2CN3)cc1 ; N#CN1CC2CCC1CN2Cc1ccc(-n2c(-c3cccnc3N)nc3ccc(-c4ccccc4)nc32)cc1]

Example 37 was prepared in a manner similar to Example 1. MS: m/z=513.2 [M+H]⁺. ¹H NMR (400 MHz, Acetonitrile-d₃) δ 8.17 (d, J=8.4 Hz, 1H), 8.04-7.99 (m, 3H), 7.89 (d, J=8.4 Hz, 1H), 7.54 (d, J=8.4 Hz, 2H), 7.48-7.45 (m, 1H), 7.44-7.37 (m, 4H), 7.14 (dd, J=7.6, 2.0 Hz, 1H), 6.58 (br s, 2H), 7.14 (dd, J=8.0, 4.8 Hz, 1H), 3.80 (m, 2H), 3.78-3.66 (m, 1H), 3.41-3.34 (m, 2H), 3.13-3.06 (m, 1H), 2.90-2.83 (m, 2H), 2.10-2.01 (m, 2H), 1.86-1.68 (m, 2H).

### Example 38: 2-(4-(2-(2-Aminopyridin-3-yl)-5-phenyl-3H-imidazo[4,5-b]pyridin-3-yl)benzyl)-2,6-diazaspiro[3.4]octane-6-carbonitrile

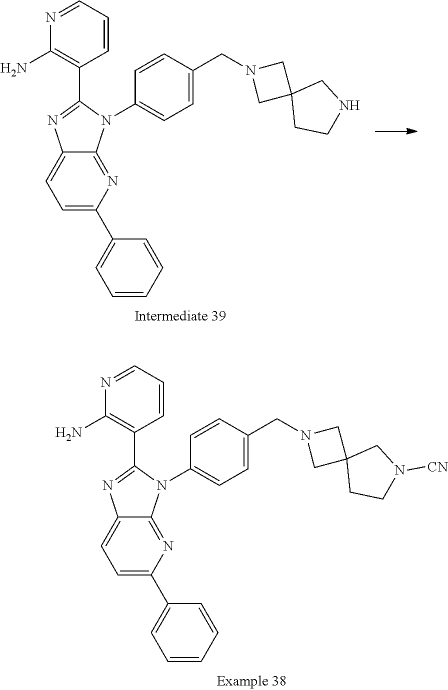 [SMILES: Nc1ncccc1-c1nc2ccc(-c3ccccc3)nc2n1-c1ccc(CN2CC3(CCNC3)C2)cc1 ; N#CN1CCC2(C1)CN(Cc1ccc(-n3c(-c4cccnc4N)nc4ccc(-c5ccccc5)nc43)cc1)C2]

Example 38 was prepared in a manner similar to Example 5. MS: m/z=513.3 [M+H]⁺. ¹H NMR (400 MHz, Methanol-d₄) δ 8.16 (d, J=8.4 Hz, 1H), 8.01 (d, J=6.8 Hz, 2H), 7.97 (dd, J=4.8, 1.6 Hz, 1H), 7.91 (d, J=8.4 Hz, 1H), 7.49-7.44 (m, 2H), 7.44-7.33 (m, 5H), 7.28 (dd, J=7.6, 1.6 Hz, 1H), 6.45 (dd, J=7.6, 4.8 Hz, 1H), 3.73 (s, 2H), 3.51 (s, 2H), 3.42 (t, J=6.8 Hz, 2H), 3.33-3.31 (m, 2H), 3.31-3.30 (m, 2H), 2.12 (t, J=6.8 Hz, 2H).

### Example 39: 2-(4-(2-(2-Aminopyridin-3-yl)-5-phenyl-3H-imidazo[4,5-b]pyridin-3-yl)benzyl)-2,6-diazaspiro[3.5]nonane-6-carbonitrile

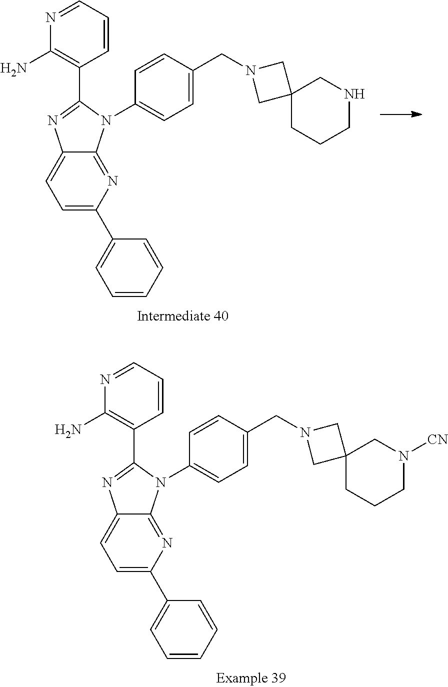 [SMILES: Nc1ncccc1-c1nc2ccc(-c3ccccc3)nc2n1-c1ccc(CN2CC3(CCCNC3)C2)cc1 ; N#CN1CCCC2(C1)CN(Cc1ccc(-n3c(-c4cccnc4N)nc4ccc(-c5ccccc5)nc43)cc1)C2]

Example 39 was prepared in a manner similar to Example 5. MS: m/z=M+H]+. ¹H NMR (400 MHz, Dimethylsulfoxide-d₆) δ 8.26 (d, J=8.0 Hz, 1H), 8.05-7.96 (m, 4H), 7.49-7.36 (m, 7H) 7.16 (dd, J=7.6, 1.6 Hz, 1H), 7.00 (br s, 2H), 6.39 (dd, J=7.6, 4.8 Hz, 1H), 3.70 (s, 2H), 3.28 (s, 2H), 3.13-3.08 (m, 4H), 2.95-2.91 (m, 2H), 1.69-1.64 (m, 2H), 1.56-1.50 (m, 2H).

### Example 40: (S)-7-(4-(2-(2-Aminopyridin-3-yl)-5-phenyl-3H-imidazo[4,5-b]pyridin-3-yl)benzyl)-2,7-diazaspiro[4.4]nonane-2-carbonitrile

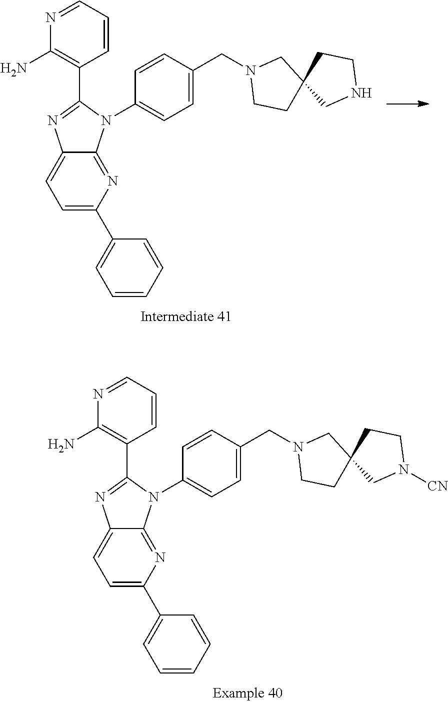 [SMILES: Nc1ncccc1-c1nc2ccc(-c3ccccc3)nc2n1-c1ccc(CN2CC[C@]3(CCNC3)C2)cc1 ; N#CN1CC[C@]2(CCN(Cc3ccc(-n4c(-c5cccnc5N)nc5ccc(-c6ccccc6)nc54)cc3)C2)C1]

Example 40 was prepared in a manner similar to Example 1. MS: m/z=527.3 [M+H]⁺. ¹H NMR (400 MHz, Methanol-d₄) δ 8.31 (d, J=8.4 Hz, 1H), 8.10-8.00 (m, 4H), 7.95-7.80 (m, 3H), 7.75-7.65 (m, 2H), 7.50-7.35 (m, 3H), 6.95-6.85 (m, 1H), 4.55-4.50 (m, 2H), 4.00-3.50 (m, 8H), 2.50-2.00 (m, 4H).

### Example 41: N-(1-(4-(2-(2-Aminopyridin-3-yl)-5-phenyl-3H-imidazo[4,5-b]pyridin-3-yl)benzyl)piperidin-4-yl)-N-(methyl-d₃)cyanamide

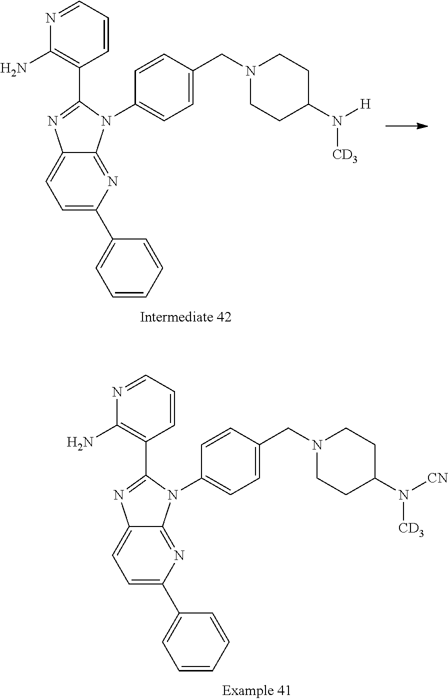 [SMILES: [2H]C([2H])([2H])NC1CCN(Cc2ccc(-n3c(-c4cccnc4N)nc4ccc(-c5ccccc5)nc43)cc2)CC1 ; [2H]C([2H])([2H])N(C#N)C1CCN(Cc2ccc(-n3c(-c4cccnc4N)nc4ccc(-c5ccccc5)nc43)cc2)CC1]

Example 41 was prepared in a manner similar to Example 1. MS: m/z=518.3 [M+H]⁺. ¹H NMR (400 MHz, Methanol-d₄) δ 8.19 (d, J=8.4 Hz, 1H), 8.05-8.02 (m, 2H), 7.98 (dd, J=4.8, 1.6 Hz, 1H), 7.94 (d, J=8.4 Hz, 1H), 7.55-7.52 (m, 2H), 7.47-7.37 (m, 5H), 7.33 (dd, J=7.6, 1.6 Hz, 1H), 6.48 (dd, J=7.6, 4.6 Hz, 1H), 3.65 (s, 2H), 3.00 (d, J=12.0 Hz, 2H), 2.93-2.86 (m, 1H), 2.23-2.16 (m, 2H), 2.03-1.90 (m, 2H), 1.75-1.71 (m, 1H), 1.69-1.64 (m, 1H).

### Example 42: N-(4-(4-(2-(2-Aminopyridin-3-yl)-5-phenyl-3H-imidazo[4,5-b]pyridin-3-yl)benzyl)piperazin-1-yl)cyanamide

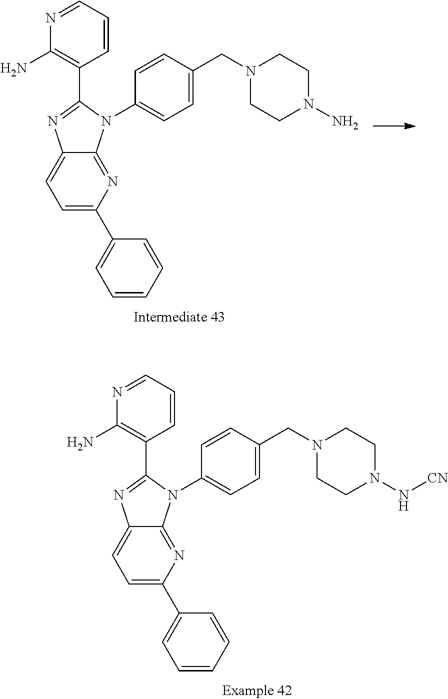 [SMILES: Nc1ncccc1-c1nc2ccc(-c3ccccc3)nc2n1-c1ccc(CN2CCN(N)CC2)cc1 ; N#CNN1CCN(Cc2ccc(-n3c(-c4cccnc4N)nc4ccc(-c5ccccc5)nc43)cc2)CC1]

Example 42 was prepared in a manner similar to Example 1. MS: m/z=. ¹H NMR (400 MHz, Methanol-d₄) δ 8.19 (d, J=8.8 Hz, 1H), 8.06-7.92 (m, 4H), 7.55-7.51 (m, 2H), 7.47-7.29 (m, 6H), 6.48 (dd, J=7.6, 4.8 Hz, 1H), 3.63 (s, 2H), 3.01-2.76 (m, 4H), 2.77-2.29 (m, 4H).

### Example 43: (3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrole-2(1H)-carbonitrile

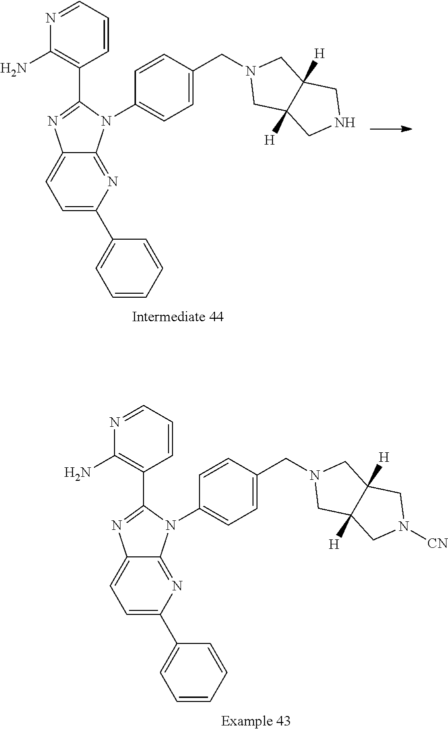 [SMILES: Nc1ncccc1-c1nc2ccc(-c3ccccc3)nc2n1-c1ccc(CN2C[C@H]3CNC[C@H]3C2)cc1 ; N#CN1C[C@@H]2CN(Cc3ccc(-n4c(-c5cccnc5N)nc5ccc(-c6ccccc6)nc54)cc3)C[C@@H]2C1]

Example 43 was prepared in a manner similar to Example 1. MS: m/z=513.3 [M+H]⁺. ¹H NMR (400 MHz, Dimethylsulfoxide-d₆) δ 8.27 (d, J=8.4 Hz, 1H), 8.05-7.95 (m, 4H), 7.52-7.41 (m, 7H), 7.15 (dd, J=7.6, 2.0 Hz, 1H), 7.02 (br s, 2H), 6.40 (dd, J=4.8, 3.2 Hz, 1H), 3.65 (br s, 2H), 3.32-3.31 (m, 2H), 3.20-3.13 (m, 2H), 2.82-2.79 (m, 2H), 2.62-2.58 (m, 2H), 2.40-2.36 (m, 2H).

### II. Biological Evaluation

### Example 1: NanoBRET Target Engagement (TE) Assay

NanoBRET is a highly specific and validated cell-based technique for assessing target engagement (Vasta et al., 2018, Cell Chem Biol. 25(2):206-214). The NanoBRET™ Target Engagement (TE) Intracellular Kinase Assays are based on the NanoBRET™ System (Promega Corporation), an energy transfer technique designed to measure molecular proximity in living cells. The NanoBRET™ TE Assays measure the apparent affinity of test compounds by competitive displacement of the NanoBRET™ tracer compound, which is a cell permeable molecule engineered to be reversibly bound to a NanoLuc® luciferase-kinase fusion expressed in cells. For compound screening, when a test compound binds to the selected kinase, the BRET signal is attenuated. For kinase inhibitors in particular, intracellular target selectivity is fundamental to pharmacological mechanism and allows the proteins of interest to be in the correct cellular confirmation. Although non-cell-based techniques have been developed to measure kinase binding or enzymatic inhibition with accuracy and precision, such approaches can fail to accurately predict engagement of the full-length target protein in the more complex and biologically relevant cellular context (Knight and Shokat, 2005, Chem. Biol. 12, 621-637; Smyth and Collins, 2009, J. Chem. Biol. 2, 131-151). The NanoBRET assay procedure was used to interrogate the compounds against the full length AKT E17K per manufacturers suggestions. Briefly, HEK-293 cells (ATCC Cat #CRL-1573) were used for transfection purposes using FuGENE HD Transfection Reagent (Promega Cat #E2311). All cells were evaluated for viability prior to transfection and optimization of the transfection was done prior to experimentation. Greater than 95% viability was used for all experiments. Following transfection, cells were washed and resuspended in Opti-MEM. NanoBRET assays were performed in white, 384-well plates (Corning) at a density of 2×10⁵ cells/well. All example compounds were prepared as concentrated stock solutions in DMSO (Sigma-Aldrich). Compounds are dissolved in DMSO to make 10 mM stock solution. Example compounds were transferred as 40 μL of 10 mM stock solution to a 384 pp-plate (LABCYTE, PP-0200) and diluted in 3-fold, 10-point dilution via transferring 12 μL compound into 24 μL DMSO by Apricot liquid handler. A Labcyte ECHO 550 compound dispenser was used to facilitate compound transfer directly to cells. Cells were equilibrated for 2 hr with energy transfer probes and example compound prior to BRET measurements. The AKTE17K (Promega Cat #NV2421) as well as specific probe (NanoBRET tracer, Promega Cat #N264B) was prepared at a concentration of 20× in tracer dilution buffer (12.5 mM HEPES, 31.25% PEG-400, pH 7.5). For target engagement analysis, the energy transfer probes were added to the cells at concentrations optimized for the target in question (AKT E17K). Following compound incubation, NanoBRET NanoGlo Substrate (Promega Cat #N157D) and Extracellular Nanoluc Inhibitor (Promega Cat #N235C) was added according to the manufacturer's recommended protocol, and luminescence was measured on Envision Reader (Perkin Elmer) Multimode Luminometer equipped with 450 nmBPfilter (donor) and 600 nmLPfilter (acceptor), using 0.5 s integration time. Milli-BRET units (mBU) are calculated by multiplying the raw BRET values by 1000. Apparent tracer affinity values (EC50) were determined using the sigmoidal dose-response (variable slope). Competitive displacement data were then plotted and data were fit to determine the EC₅₀ value for each example compound. Table 2 provides the assay results for select examples. Activity is defined as “+”, for EC₅₀ greater 600 nanomolar; “++” for EC₅₀ between 60-600 nanomolar; “+++” for EC₅₀ between 15-60 nanomolar; and “++++”, for EC₅₀ less than 15 nanomolar.

TABLE 2

| Example | MS: | AKT1-E17K | |
|---|---|---|---|
| Number | [M + H]+ | IC50 (nM) | Name |
| 1 | 425.1 | + | N-(1-(4-(2-(2-aminopyridin-3-yl)-3H-imidazo[4,5- |
| | | | b]pyridin-3-yl)benzyl)piperidin-4-yl)cyanamide |
| 2 | 487.1 | + | 4-(4-(2-(2-aminopyridin-3-yl)-5-phenyl-3H- |
| | | | imidazo[4,5-b]pyridin-3-yl)benzyl)piperazine-1- |
| | | | carbonitrile |
| 3 | 501.3 | ++++ | N-(1-(4-(2-(2-aminopyridin-3-yl)-5-phenyl-3H- |
| | | | imidazo[4,5-b]pyridin-3-yl)benzyl)piperidin-4- |
| | | | yl)cyanamide |
| 4 | 558.1 | ++++ | N-(3-(2-(2-aminopyridin-3-yl)-3-(4-((4- |
| | | | cyanamidopiperidin-1-yl)methyl)phenyl)-3H- |
| | | | imidazo[4,5-b]pyridin-5-yl)phenyl)acetamide |
| 5 | 499.2 | +++ | 6-(4-(2-(2-aminopyridin-3-yl)-5-phenyl-3H- |
| | | | imidazo[4,5-b]pyridin-3-yl)benzyl)-2,6- |
| | | | diazaspiro[3.3]heptane-2-carbonitrile |
| 6 | 501.3 | +++ | (S)-N-(1-(4-(2-(2-aminopyridin-3-yl)-5-phenyl-3H- |
| | | | imidazo[4,5-b]pyridin-3-yl)benzyl)piperidin-3- |
| | | | yl)cyanamide |
| 7 | 487.2 | ++++ | (S)-N-(1-(4-(2-(2-aminopyridin-3-yl)-5-phenyl-3H- |
| | | | imidazo[4,5-b]pyridin-3-yl)benzyl)pyrrolidin-3- |
| | | | yl)cyanamide |
| 8 | 555.3 | ++ | N-(2-(4-(2-(2-aminopyridin-3-yl)-5-phenyl-3H- |
| | | | imidazo[4,5-b]pyridin-3-yl)benzyl)-2- |
| | | | azaspiro[4.5]decan-8-yl)cyanamide |
| 9 | 501.3 | +++ | (R)-N-(1-(4-(2-(2-aminopyridin-3-yl)-5-phenyl-3H- |
| | | | imidazo[4,5-b]pyridin-3-yl)benzyl)piperidin-3- |
| | | | yl)cyanamide |
| 10 | 487.4 | ++++ | (R)-N-(1-(4-(2-(2-aminopyridin-3-yl)-5-phenyl-3H- |
| | | | imidazo[4,5-b]pyridin-3-yl)benzyl)pyrrolidin-3- |
| | | | yl)cyanamide |
| 11 | 501.1 | ++ | N-(1-(4-(2-(2-aminopyridin-3-yl)-6-phenyl-3H- |
| | | | imidazo[4,5-b]pyridin-3-yl)benzyl)piperidin-4- |
| | | | yl)cyanamide |
| 12 | 501.2 | ++ | 4-(4-(2-(2-aminopyridin-3-yl)-5-phenyl-3H- |
| | | | imidazo[4,5-b]pyridin-3-yl)benzyl)-1,4-diazepane-1- |
| | | | carbonitrile |
| 13 | 541.3 | ++++ | N-(7-(4-(2-(2-aminopyridin-3-yl)-5-phenyl-3H- |
| | | | imidazo[4,5-b]pyridin-3-yl)benzyl)-7- |
| | | | azaspiro[3.5]nonan-2-yl)cyanamide |
| 14 | 515.3 | +++ | N-(1-(4-(2-(2-aminopyridin-3-yl)-5-phenyl-3H- |
| | | | imidazo[4,5-b]pyridin-3-yl)benzyl)piperidin-4-yl)- |
| | | | N-methylcyanamide |
| 15 | 527.3 | +++ | 7-(4-(2-(2-aminopyridin-3-yl)-5-phenyl-3H- |
| | | | imidazo[4,5-b]pyridin-3-yl)benzyl)-2,7- |
| | | | diazaspiro[3.5]nonane-2-carbonitrile |
| 16 | 541.4 | ++++ | 8-(4-(2-(2-aminopyridin-3-yl)-5-phenyl-3H- |
| | | | imidazo[4,5-b]pyridin-3-yl)benzyl)-2,8- |
| | | | diazaspiro[4.5]decane-2-carbonitrile |
| 17 | 555.4 | +++ | 9-(4-(2-(2-aminopyridin-3-yl)-5-phenyl-3H- |
| | | | imidazo[4,5-b]pyridin-3-yl)benzyl)-3,9- |
| | | | diazaspiro[5.5]undecane-3-carbonitrile |
| 18 | 513.3 | ++ | 6-(4-(2-(2-aminopyridin-3-yl)-5-phenyl-3H- |
| | | | imidazo[4,5-b]pyridin-3-yl)benzyl)-2,6- |
| | | | diazaspiro[3.4]octane-2-carbonitrile |
| 19 | 527.3 | +++ | 7-(4-(2-(2-aminopyridin-3-yl)-5-phenyl-3H- |
| | | | imidazo[4,5-b]pyridin-3-yl)benzyl)-2,7- |
| | | | diazaspiro[4.4]nonane-2-carbonitrile |
| 20 | 541.4 | +++ | 2-(4-(2-(2-aminopyridin-3-yl)-5-phenyl-3H- |
| | | | imidazo[4,5-b]pyridin-3-yl)benzyl)-2,8- |
| | | | diazaspiro[4.5]decane-8-carbonitrile |
| 21 | 541.4 | + | 2-(4-(2-(2-aminopyridin-3-yl)-5-phenyl-3H- |
| | | | imidazo[4,5-b]pyridin-3-yl)benzyl)-2,7- |
| | | | diazaspiro[4.5]decane-7-carbonitrile |
| 22 | 527.3 | ++ | 2-(4-(2-(2-aminopyridin-3-yl)-5-phenyl-3H- |
| | | | imidazo[4,5-b]pyridin-3-yl)benzyl)-2,7- |
| | | | diazaspiro[3.5]nonane-7-carbonitrile |
| 23 | 527.2 | ++++ | (R)-7-(4-(2-(2-aminopyridin-3-yl)-5-phenyl-3H- |
| | | | imidazo[4,5-b]pyridin-3-yl)benzyl)-2,7- |
| | | | diazaspiro[4.4]nonane-2-carbonitrile |
| 24 | 555.4 | ++ | 9-(4-(2-(2-aminopyridin-3-yl)-5-phenyl-3H- |
| | | | imidazo[4,5-b]pyridin-3-yl)benzyl)-2,9- |
| | | | diazaspiro[5.5]undecane-2-carbonitrile |
| 25 | 515.3 | ++ | (2S,6S)-4-(4-(2-(2-aminopyridin-3-yl)-5-phenyl-3H- |
| | | | imidazo[4,5-b]pyridin-3-yl)benzyl)-2,6- |
| | | | dimethylpiperazine-1-carbonitrile |
| 26 | 515.3 | + | (2R,6R)-4-(4-(2-(2-aminopyridin-3-yl)-5-phenyl- |
| | | | 3H-imidazo[4,5-b]pyridin-3-yl)benzyl)-2,6- |
| | | | dimethylpiperazine-1-carbonitrile |
| 27 | 501.2 | + | (R)-4-(4-(2-(2-aminopyridin-3-yl)-5-phenyl-3H- |
| | | | imidazo[4,5-b]pyridin-3-yl)benzyl)-2- |
| | | | methylpiperazine-1-carbonitrile |
| 28 | 513.3 | ++ | 3-(4-(2-(2-aminopyridin-3-yl)-5-phenyl-3H- |
| | | | imidazo[4,5-b]pyridin-3-yl)benzyl)-3,8- |
| | | | diazabicyclo[3.2.1]octane-8-carbonitrile |
| 29 | 513.3 | +++ | 8-(4-(2-(2-aminopyridin-3-yl)-5-phenyl-3H- |
| | | | imidazo[4,5-b]pyridin-3-yl)benzyl)-3,8- |
| | | | diazabicyclo[3.2.1]octane-3-carbonitrile |
| 30 | 515.2 | +++ | N-(1-(4-(2-(2-aminopyridin-3-yl)-5-phenyl-3H- |
| | | | imidazo[4,5-b]pyridin-3-yl)benzyl)azepan-4- |
| | | | yl)cyanamide |
| 31 | 515.4 | ++ | N-(1-(4-(2-(2-Aminopyridin-3-yl)-5-phenyl-3H- |
| | | | imidazo[4,5-b]pyridin-3-yl)benzyl)azepan-3- |
| | | | yl)cyanamide |
| 32 | 501.2 | + | (S)-4-(4-(2-(2-aminopyridin-3-yl)-5-phenyl-3H- |
| | | | imidazo[4,5-b]pyridin-3-yl)benzyl)-2- |
| | | | methylpiperazine-1-carbonitrile |
| 33 | 513.3 | ++ | 7-(4-(2-(2-aminopyridin-3-yl)-5-phenyl-3H- |
| | | | imidazo[4,5-b]pyridin-3-yl)benzyl)-4,7- |
| | | | diazaspiro[2.5]octane-4-carbonitrile |
| 34 | 499.3 | ++ | (1S,4S)-5-(4-(2-(2-aminopyridin-3-yl)-5-phenyl-3H- |
| | | | imidazo[4,5-b]pyridin-3-yl)benzyl)-2,5- |
| | | | diazabicyclo[2.2.1]heptane-2-carbonitrile |
| 35 | 499.3 | ++ | (1R,4R)-5-(4-(2-(2-aminopyridin-3-yl)-5-phenyl- |
| | | | 3H-imidazo[4,5-b]pyridin-3-yl)benzyl)-2,5- |
| | | | diazabicyclo[2.2.1]heptane-2-carbonitrile |
| 36 | 515.4 | + | (2S,6R)-4-(4-(2-(2-aminopyridin-3-yl)-5-phenyl- |
| | | | 3H-imidazo[4,5-b]pyridin-3-yl)benzyl)-2,6- |
| | | | dimethylpiperazine-1-carbonitrile |
| 37 | 513.2 | ++ | 5-(4-(2-(2-aminopyridin-3-yl)-5-phenyl-3H- |
| | | | imidazo[4,5-b]pyridin-3-yl)benzyl)-2,5- |
| | | | diazabicyclo[2.2.2]octane-2-carbonitrile |
| 38 | 513.3 | ++++ | 2-(4-(2-(2-aminopyridin-3-yl)-5-phenyl-3H- |
| | | | imidazo[4,5-b]pyridin-3-yl)benzyl)-2,6- |
| | | | diazaspiro[3.4]octane-6-carbonitrile |
| 39 | 527.3 | ++ | 2-(4-(2-(2-aminopyridin-3-yl)-5-phenyl-3H- |
| | | | imidazo[4,5-b]pyridin-3-yl)benzyl)-2,6- |
| | | | diazaspiro[3.5]nonane-6-carbonitrile |
| 40 | 527.3 | ++++ | (S)-7-(4-(2-(2-aminopyridin-3-yl)-5-phenyl-3H- |
| | | | imidazo[4,5-b]pyridin-3-yl)benzyl)-2,7- |
| | | | diazaspiro[4.4]nonane-2-carbonitrile |
| 41 | 518.3 | +++ | N-(1-(4-(2-(2-aminopyridin-3-yl)-5-phenyl-3H- |
| | | | imidazo[4,5-b]pyridin-3-yl)benzyl)piperidin-4-yl)- |
| | | | N-(methyl-d3)cyanamide |
| 42 | 524.1 | ++++ | N-(4-(4-(2-(2-aminopyridin-3-yl)-5-phenyl-3H- |
| | [M + Na]⁺ | | imidazo[4,5-b]pyridin-3-yl)benzyl)piperazin-1- |
| | | | yl)cyanamide |
| 43 | 513.3 | ++ | (3aR,6aS)-5-(4-(2-(2-aminopyridin-3-yl)-5-phenyl- |
| | | | 3H-imidazo[4,5-b]pyridin-3- |
| | | | yl)benzyl)hexahydropyrrolo[3,4-c]pyrrole-2(1H)- |
| | | | carbonitrile |

### III. Preparation of Pharmaceutical Dosage Forms

### Example 1: Oral capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

We claim:

1. A compound having the structure of Formula (I), or a pharmaceutically acceptable salt or solvate thereof: 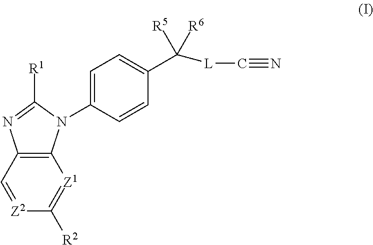
wherein:
Z¹ is N, C—H, or C—R³;
Z² is N, C—H, or C—R⁴;
R¹ is selected from hydrogen, halogen, —CN, optionally substituted C1-C6 alkyl, optionally substituted aryl, or optionally substituted heteroaryl;
R² is selected from hydrogen, halogen, —CN, optionally substituted C1-C6 alkyl, optionally substituted aryl, or optionally substituted heteroaryl;
R³ is selected from halogen, —CN, optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
L is selected from —N(R⁷)—, or a divalent radical selected from: 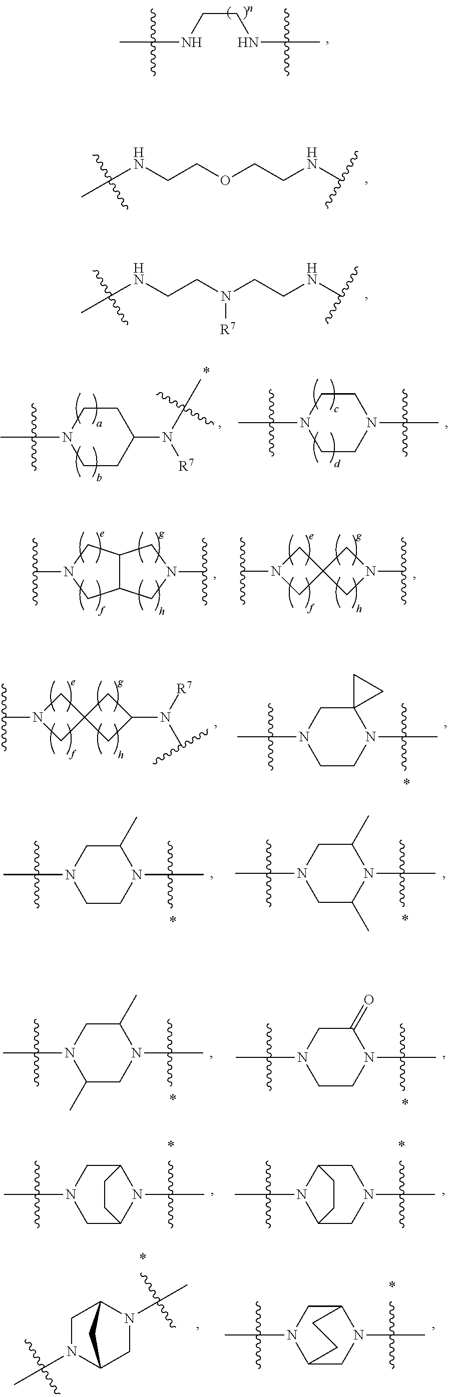 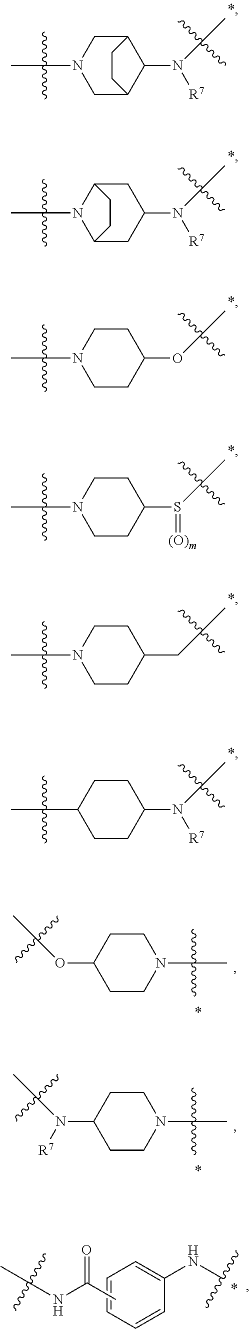 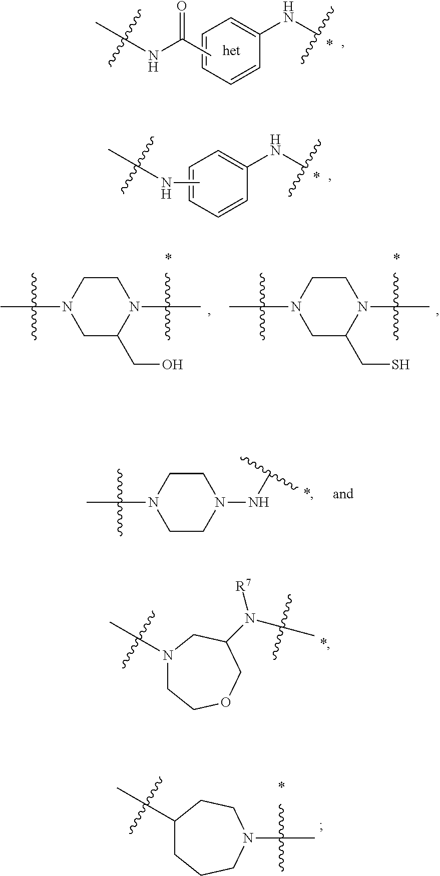
wherein the asterisk (*) indicates the bond to the cyanamide group;
a is 0, 1, 2, 3, or 4;
b is 0, 1, 2, 3, or 4;
c is 1, 2, 3, or 4;
d is 1, 2, 3, or 4;
e is 0, 1, 2, 3, or 4;
f is 0, 1, 2, 3, or 4; provided that e and f are not both 0;
g is 0, 1, 2, 3, or 4; provided that e and g are not both 0;
h is 0, 1, 2, 3, or 4; provided that g and h are not both 0; and provided that f and h are not both 0;
m is 0, 1, or 2;
n is 1, 2, or 3; and
R⁷ is hydrogen, optionally substituted C1-C6 alkyl, optionally substituted C3-C7 cycloalkyl, or optionally substituted heterocyclyl.

2. The compound of claim 1, or pharmaceutically acceptable salt or solvate thereof, wherein Z¹ is N.

3. The compound of any one of claims 1-2, or pharmaceutically acceptable salt or solvate thereof, wherein Z² is C—H.

4. The compound of any one of claims 1-2, or pharmaceutically acceptable salt or solvate thereof, wherein Z² is C—R⁴.

5. The compound of any one of claims 1-4, or pharmaceutically acceptable salt or solvate thereof, wherein R¹ is optionally substituted heteroaryl.

6. The compound of claim 5, or pharmaceutically acceptable salt or solvate thereof, wherein the optionally substituted heteroaryl is an optionally substituted pyridyl.

7. The compound of any one of claims 1-6, or pharmaceutically acceptable salt or solvate thereof, wherein R² is optionally substituted aryl.

8. The compound of claim 7, or pharmaceutically acceptable salt or solvate thereof, wherein the optionally substituted aryl is an optionally substituted phenyl.

9. The compound of any one of claims 1-6, or pharmaceutically acceptable salt or solvate thereof, wherein R² is hydrogen.

10. The compound of claim 4, or pharmaceutically acceptable salt or solvate thereof, wherein Z¹ is N; R² is hydrogen; and R⁴ is an optionally substituted phenyl.

11. The compound of any one of claims 1, or 3-9, or pharmaceutically acceptable salt or solvate thereof, wherein R³ is optionally substituted C1-C6 alkyl.

12. The compound of any one of claims 1, or 3-9, or pharmaceutically acceptable salt or solvate thereof, wherein R³ is optionally substituted aryl.

13. The compound of any one of claims 1, 2, or 4-9, or pharmaceutically acceptable salt or solvate thereof, wherein R⁴ is optionally substituted C1-C6 alkyl.

14. The compound of any one of claims 1, 2, or 4-9, or pharmaceutically acceptable salt or solvate thereof, wherein R⁴ is optionally substituted aryl.

15. The compound of any one of claims 1-14, or pharmaceutically acceptable salt or solvate thereof, wherein R⁵ is hydrogen.

16. The compound of any one of claims 1-15, or pharmaceutically acceptable salt or solvate thereof, wherein R⁶ is hydrogen.

17. The compound of any one of claims 1-14, or pharmaceutically acceptable salt or solvate thereof, wherein R⁵ and R⁶ together form an oxo.

18. The compound of any one of claims 1-15, or pharmaceutically acceptable salt or solvate thereof, wherein R⁶ is optionally substituted C1-C6 alkyl.

19. The compound of any one of claims 1-14, or pharmaceutically acceptable salt or solvate thereof, wherein R⁵ and R⁶ join together to form a carbocycle or heterocycle.

20. The compound of any one of claims 1-19, or pharmaceutically acceptable salt or solvate thereof, wherein L is —N(R⁷)—.

21. The compound of any one of claims 1-19, or pharmaceutically acceptable salt or solvate thereof, wherein L is selected from:

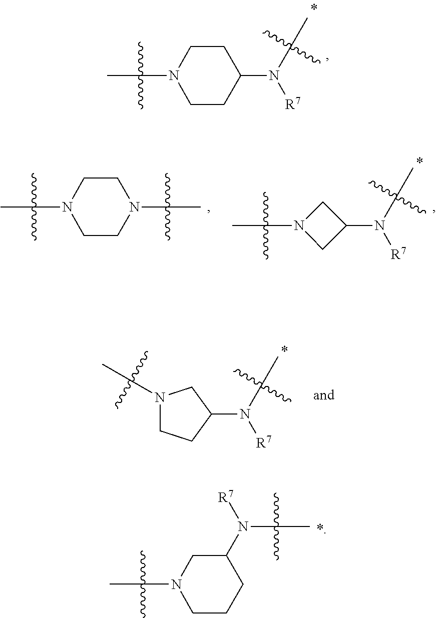

22. The compound of any one of claims 1-19, or pharmaceutically acceptable salt or solvate thereof, wherein L is selected from:

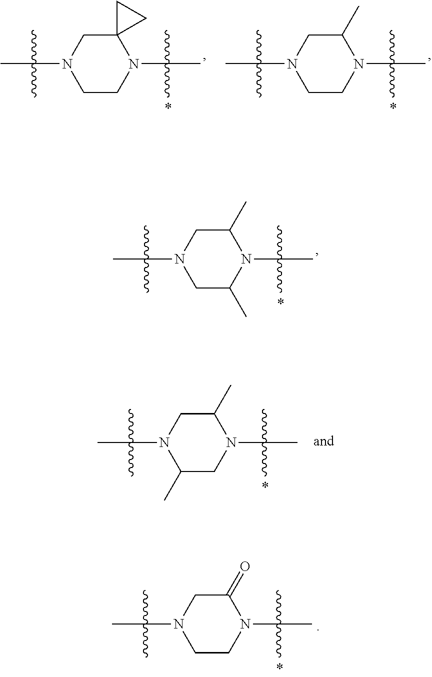

23. The compound of any one of claims 1-19, or pharmaceutically acceptable salt or solvate thereof, wherein L is selected from:

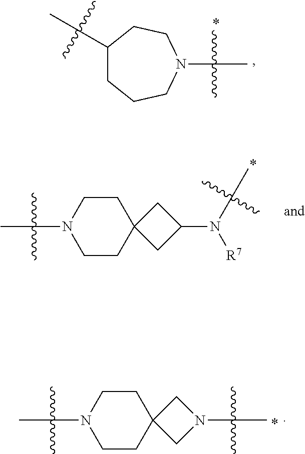

24. The compound of any one of claims 1-19, or pharmaceutically acceptable salt or solvate thereof, wherein L is selected from:

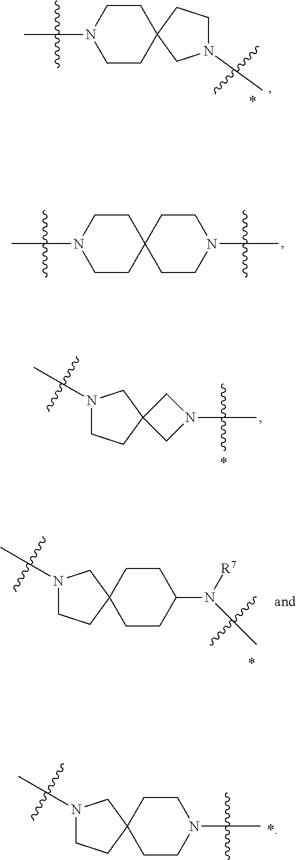

25. The compound of any one of claims 1-19, or pharmaceutically acceptable salt or solvate thereof, wherein L is selected from:

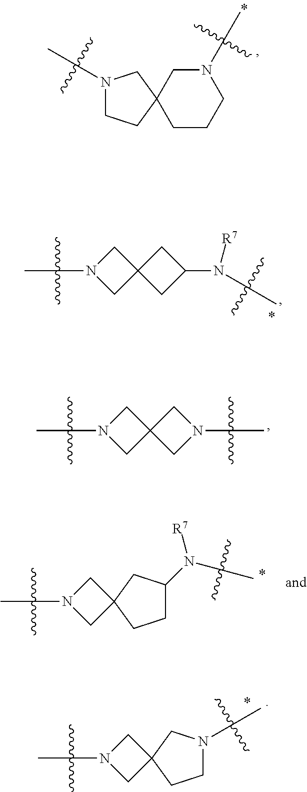

26. The compound of any one of claims 1-19, or pharmaceutically acceptable salt or solvate thereof, wherein L is selected from:

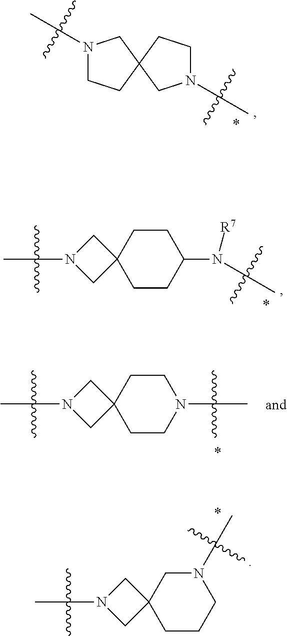

27. The compound of any one of claims 1-19, or pharmaceutically acceptable salt or solvate thereof, wherein L is

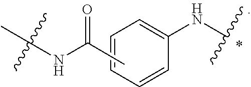

28. The compound of claim 27, or pharmaceutically acceptable salt or solvate thereof, wherein L is

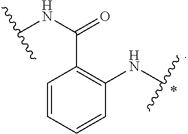 [SMILES: *NC(=O)c1ccccc1N*]

29. The compound of claim 27, or pharmaceutically acceptable salt or solvate thereof, wherein L is

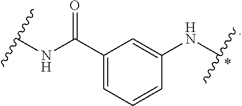 [SMILES: *NC(=O)c1cccc(N*)c1]

30. The compound of claim 27, or pharmaceutically acceptable salt or solvate thereof, wherein L is

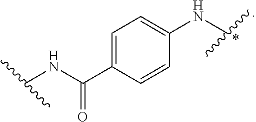 [SMILES: *NC(=O)c1ccc(N*)cc1]

31. The compound of any one of claims 1-19, or pharmaceutically acceptable salt or solvate thereof, wherein L is

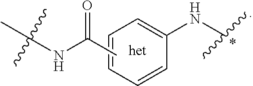

32. The compound of claim 31, or pharmaceutically acceptable salt or solvate thereof, wherein L is

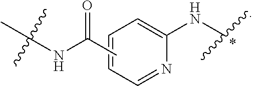

33. The compound of claim 31, or pharmaceutically acceptable salt or solvate thereof, wherein L is

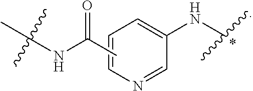

34. The compound of claim 31, or pharmaceutically acceptable salt or solvate thereof, wherein L is

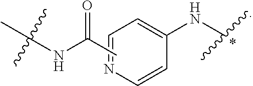

35. The compound of claim 31, or pharmaceutically acceptable salt or solvate thereof, wherein L is

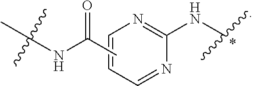

36. The compound of claim 31, or pharmaceutically acceptable salt or solvate thereof, wherein L is

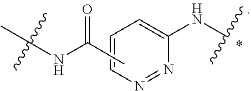

37. The compound of claim 31, or pharmaceutically acceptable salt or solvate thereof, wherein L is

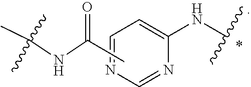

38. The compound of claim 31, or pharmaceutically acceptable salt or solvate thereof, wherein L is

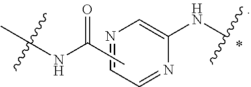

39. The compound of claim 31, or pharmaceutically acceptable salt or solvate thereof, wherein L is

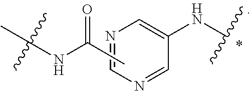

40. The compound of any one of claims 1-19, or pharmaceutically acceptable salt or solvate thereof, where L is

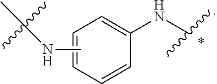

41. The compound of claim 40, or pharmaceutically acceptable salt or solvate thereof, wherein L is

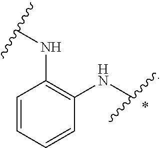 [SMILES: *Nc1ccccc1N*]

42. The compound of claim 40, or pharmaceutically acceptable salt or solvate thereof, wherein L is

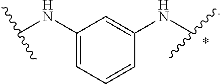 [SMILES: *Nc1cccc(N*)c1]

43. The compound of claim 40, or pharmaceutically acceptable salt or solvate thereof, wherein L is

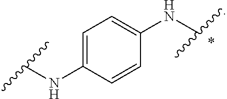 [SMILES: *Nc1ccc(N*)cc1]

44. The compound of any one of claims 1-43, or pharmaceutically acceptable salt or solvate thereof, wherein R⁷ is hydrogen or optionally substituted C1-C6 alkyl.

45. A compound, or pharmaceutically acceptable salt or solvate thereof, as described in Table 1.

46. A pharmaceutical composition comprising a compound, or pharmaceutically acceptable salt or solvate thereof, as described in any one of claims 1-45 and a pharmaceutically acceptable excipient.

47. A method of preparing a pharmaceutical composition comprising mixing a compound, or pharmaceutically acceptable salt or solvate thereof, of any one of claims 1-45, and a pharmaceutically acceptable carrier.

48. A compound of any one of claims 1-45, or pharmaceutically acceptable salt or solvate thereof, for use in a method of treatment of the human or animal body.

49. A compound of any one of claims 1-45, or pharmaceutically acceptable salt or solvate thereof, for use in a method of treatment of cancer or neoplastic disease.

50. Use of a compound of any one of claims 1-45, or pharmaceutically acceptable salt or solvate thereof, in the manufacture of a medicament for the treatment of cancer or neoplastic disease.

51. A method of treating cancer in a patient in need thereof, comprising administering to the patient a compound as described in any one of claims 1-45, or pharmaceutically acceptable salt or solvate thereof.

52. A method of treating cancer in a patient in need thereof, comprising administering to the patient a pharmaceutical composition comprising a compound as described in any one of claims 1-45, or pharmaceutically acceptable salt or solvate thereof, and a pharmaceutically acceptable excipient.

53. A method of inhibiting an AKT1 enzyme comprising contacting the enzyme with a compound of any one of claims 1-45, wherein the AKT1 enzyme is contacted in an in vitro setting.

54. A method of inhibiting an AKT1 enzyme comprising contacting the enzyme with a compound of any one of claims 1-45, wherein the AKT1 enzyme is contacted in an in vivo setting.
